(19) United States
(12) Patent Application Publication
(10) Pub. No.: US 2007/0186290 A1
(43) Pub. Date: Aug. 9, 2007

(54) Diagnostic and therapeutic use of the human hif3alpha gene and proteins for neurodegenerative diseases

(76) Inventors: Heinz Von Der Kammer, Hamburg (DE); Johannes Pohlner, Hamburg (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 10/596,627

(22) PCT Filed: Dec. 17, 2004
(86) PCT No.: PCT/EP04/53573

§ 371 Date: Jun. 19, 2006

Related U.S. Application Data
(60) Provisional application No. 60/530,227, filed on Dec. 18, 2003.

(51) Int. Cl. A01K 67/033; A01K 67/027; C12Q 1/68; G01N 33/567

(52) U.S. Cl. 800/12; 435/7.2; 435/6; 514/44

(57) ABSTRACT

The present invention discloses the differential expression of a gene coding for HIF3a in specific brain regions of Alzheimer's disease patients. Based on this finding, the invention provides a method for diagnosing or prognosticating a neurodegen-erative disease, in particular Alzheimer's disease, in a subject, or for determining whether a subject is at increased risk of developing such a disease. Furthermore, this invention provides therapeutic and prophylactic methods for treating or preventing Alzheimer's disease and related neurodegenerative disorders using the HIF3a gene and its corresponding gene products. A method of screening for modulating agents of neurodegenerative diseases is also disclosed.

The present invention relates to methods of diagnosing, prognosticating, and monitoring the progression of neurodegenerative diseases in a subject. Furthermore, methods of therapy control and screening for modulating agents of neurodegenerative diseases are provided. The invention also discloses pharmaceutical compositions, kits, and recombinant animal models.

Neurodegenerative diseases, in particular Alzheimer's disease (AD), have a strongly debilitating impact on a patient's life. Furthermore, these diseases constitute an enormous health, social, and economic burden. AD is the most common neurodegenerative disease, accounting for about 70% of all dementia cases, and it is probably the most devastating age-related neurodegenerative condition affecting about 10% of the population over 65 years of age and up to 45% over age 85 (for a recent review see Vickers et al., Progress in Neurobiology 2000, 60: 139-165). Presently, this amounts to an estimated 12 million cases in the U.S., Europe, and Japan. This situation will inevitably worsen with the demographic increase in the number of old people (“aging of the baby boomers”) in developed countries. The neuropathological hallmarks that occur in the brains of individuals with AD are senile plaques, composed of amyloid-β protein, and profound cytoskeletal changes coinciding with the appearance of abnormal filamentous structures and the formation of neurofibrillary tangles. The amyloid-β (Aβ) protein evolves from the cleavage of the amyloid precursor protein (APP) by different kinds of proteases. The cleavage by the β/γ-secretase leads to the formation of Aβ peptides of different lengths, typically a short more soluble and slow aggregating peptide consisting of 40 amino acids and a longer 42 amino acid peptide, which rapidly aggregates outside the cells, forming the characteristic amyloid plaques (Selkoe, Physiological Rev 2001, 81: 741-66; Greenfield et al., Frontiers Bioscience 2000, 5: D72-83). They are primarily found in the cerebral cortex and hippocampus. The generation of toxic Aβ deposits in the brain starts very early in the course of AD, and it is discussed to be a key player for the subsequent destructive processes leading to AD pathology. The other pathological hallmarks of AD are neurofibrillary tangles (NFTs) and abnormal neurites, described as neuropil threads (Braak and Braak, Acta Neuropathol 1991, 82: 239-259). NFTs emerge inside neurons and consist of chemically altered tau, which forms paired helical filaments twisted around each other. The appearance of neurofibrillary tangles and their increasing number correlates well with the clinical severity of AD (Schmitt et al., Neurology 2000, 55: 370-376).

AD is a progressive disease that is associated with early deficits in memory formation and ultimately leads to the complete erosion of higher cognitive function. The cognitive disturbances include among other things memory impairment, aphasia, agnosia and the loss of executive functioning. A characteristic feature of the pathogenesis of AD is the selective vulnerability of particular brain regions and subpopulations of nerve cells to the degenerative process. Specifically, the temporal lobe region and the hippocampus are affected early and more severely during the progression of the disease. On the other hand, neurons within the frontal cortex, occipital cortex, and the cerebellum remain largely intact and are protected from neurodegeneration (Terry et al., Annals of Neurology 1981, 10: 184-92). The age of onset of AD may vary within a range of 50 years, with early-onset AD occurring in people younger than 65 years of age, and late-onset of AD occurring in those older than 65 years.

Currently, there is no cure for AD, nor is there an effective treatment to halt the progression of AD or even to diagnose AD ante-mortem with high probability. Several risk factors have been identified that predispose an individual to develop AD, among them most prominently the epsilon 4 allele of the three different existing alleles (epsilon 2, 3, and 4) of the apolipoprotein E gene (ApoE) (Strittmatter et al., Proc Natl Acad Sci USA 1993, 90: 1977-81; Roses, Ann NY Acad Sci 1998, 855: 738-43). The polymorphic plasmaprotein ApoE plays a role in the intercellular cholesterol and phospholipid transport by binding low-density lipoprotein receptors, and it seems to play a role in neurite growth and regeneration. Efforts to detect further susceptibility genes and disease-linked polymorphisms lead to the assumption that specific regions and genes on human chromosomes 10 and 12 may be associated with late-onset AD (Myers et al., Science 2000, 290: 2304-5; Bertram et al., Science 2000, 290: 2303; Scott et al., Am J Hum Genet 2000, 66: 922-32). Although there are rare examples of early-onset AD which have been attributed to genetic defects in the genes for amyloid precursor protein (APP) on chromosome 21, presenilin-1 on chromosome 14, and presenilin-2 on chromosome 1, the prevalent form of late-onset sporadic AD is of hitherto unknown etiologic origin.

The late onset and complex pathogenesis of neurodegenerative disorders pose a formidable challenge to the development of therapeutic and diagnostic agents. It is pivotal to expand the pool of potential drug targets and diagnostic markers. It is therefore an object of the present invention to provide insight into the pathogenesis of neurological diseases and to provide methods, materials, agents, compositions, and animal models which are suited inter alia for the diagnosis and development of a treatment of these diseases. This object has been solved by the features of the independent claims. The subclaims define preferred embodiments of the present invention.

The present invention is based on the detection and dysregulated, differential expression of a gene coding for a hypoxia-inducible factor 3 (HIF3α, HIF3alpha, HIF-3 alpha), alias HIF3a, and of the protein products in human Alzheimer's disease brain samples. The hypoxia-inducible factors (HIFs) belong to the growing number of proteins containing a ‘PAS’ domain. The abbreviation ‘PAS’ comes from the three protein-families PER (protein of the Drosophila Period gene), ARNT (AHR nuclear translocator) and SIM (protein of the Drosophila Single-minded locus). The PAS domain participates either in homotypic interactions with other PAS proteins or in heterotypic interactions with chaperones. Most often a basic-helix-loop-helix motif (bHLH) is found N-terminal to the PAS domain, which functions as a homotypic dimerization domain for other bHLH-PAS proteins. Both domains confer DNA binding and dimerization specificity (Jiang et al., J. Biol. Chem. 1996, 271: 17771-17778). At their C-terminus PAS proteins may contain transcriptionally active domains, for example one or more hypoxia responsive domains (HRDs). PAS proteins are implicated in various signal transduction pathways and play a role in the adaptation to environmental changes, as for example changes in atmospheric and cellular oxygen which is mediated by the hypoxia-inducible factor (HIF) system (Gu et al., Ann. Rev. Pharmacol. Toxicol. 2000, 40: 519-561). HIFs belong to the superfamily of bHLH-PAS proteins and are heterodimeric transcription factors (TFs) composed of α- and β-subunits. For the α-class subunits of the HIF1- and HIF2-TFs it has been shown that their expression levels are upregulated in response to cellular hypoxia, iron chelators, reactive oxygen species (ROS), transition metals, and exposure to divalent cations (i.e. Co²⁺ and others). These agents stabilize the α-subunit protein, thus allowing its dimerization with the β-subunit and hence the formation of a transcriptionally active HIF DNA-binding complex (Hogenesch et al., J. Biol. Chem. 1997, 271: 8581-8593 and Wang et al., Proc. Natl. Acad. Sci. USA 1995, 92: 5510-5514). The HIFα-subunits are continuously synthesized, but are present only in hypoxic cells due to a rapid degradation by the ubiquitin-proteasome system under normoxic conditions. Therefore, the hypoxic regulation of the α-class proteins guides the formation of the transcriptionally active HIF-complex. The β-subunits are constitutively expressed and interact with the corresponding α-subunit to form a complex which is translocated to the nucleus. A well studied β-subunit, HIF1β, is the aryl hydrocarbon receptor nuclear translocator (ARNT) (Wood et al., J. Biol. Chem. 1996, 271: 15117-15123 and Wang et al., Proc. Natl. Acad. Sci. USA 1995, 92: 5510-5514). Several hypoxia sensitive genes have been identified, They are regulated by binding of the heterodimeric HIF complexes to hypoxia responsive elements (HREs) which are located 5′ or 3′ to the gene promotor of the respective gene. Such HIF-regulated gene products are for example the peptide hormone erythropoietin (EPO), which is responsible for the regulation of erythropoiesis, angiogenic factors like the vascular endothelial growth factor (VEGF), the platelet-derived growth factor (PDGF), and the fibroblast growth factor (FGF), various glycolytic enzymes and glucose transporters such as GLUT1, which are involved in energy metabolism (Gu et al., Ann. Rev. Pharmacol. Toxicol. 2000, 40: 519-561). HIF proteins interact with non-PAS containing proteins as well, for instance with p53 which plays a role in hypoxia-induced apoptosis. Further, HIF1α interacts with the von Hippel-Lindau tumor suppressor gene product (pVHL) resulting in proteasome degradation (Maxwell et al., Nature 1999, 399: 271-275). In the presence of oxygen pVHL hydoxylates and targets the α-class protein of the HIF complex for polyubiquitination (Ivan et al., Proc. Natl. Acad. Sci. USA 2002, 99: 13459-13464 and Chan et al., J. Biol. Chem. 1999, 274: 12115-12123). The mechanisms of hypoxic regulation are currently based on the well studied HIF1α protein, but it is assumed that other known HIFα-family members are similarly regulated (Semenza and Wang, Mol. Cell. Biol. 1992, 12: 5447-5454).

It is well known that the human brain is highly dependent on oxygen. Constituting just approximately 2% of the whole body mass, the brain utilizes 20% of the respiratory oxygen uptake. Within minutes, oxygen deprivation leads to damages within the brain. A recently published review outlines how hypoxia causese progressive dysfunction, apoptosis, necrosis, and brain cell death (Bazan et al., Mol. Neurobiol. 2002, 26: 283-298). To date, three members of the alpha family of the HIF proteins have been identified, HIF1α, HIF2α alias endothelial PAS protein 1 (EPAS-1) or member of PAS family 2 (MOP2), and HIF3α. HIF2α is highly homologous to HIF1α in the bHLH-PAS domain, dimerizes with HIF1β, and was found to activate the hypoxia responsive VEGF promotor. In contrast to the widespread expression of HIF1α, HIF2α is mainly expressed in endothelial cells (Tian et al., Genes Dev. 1997, 11: 72-82 and Hbgenesch et al., J. Biol. Chem. 1997, 271: 8581-8593). A region of about 15 amino acids, which corresponds to amino acids 557-571 in the human HIF1α subunit, shows strong conservation among all members of the HIFα proteins. Srinivas et al. speculated that this conserved sequence is involved in the stabilization of HIFα proteins under hypoxic conditions and thus may guide hypoxia regulation (Srinivas et al., Biochem. Biophys. Res. Comm. 1999, 260: 557-561).

Very recently, in an attempt to identify new bHLH-PAS proteins, a novel α-class hypoxia-inducible factor cDNA, HIF3α, was cloned by Gu and coworkers (Gu et al., Gene Expression 1998, 7: 205-213). A mouse EST clone (GenBank accession number AA028416) was identified, based on similarities to a part of the human HIF1α gene. The mouse EST turned out to be part of a complete open reading frame of the mouse HIF3α cDNA (Genbank accession number AF060194), which spans 1.98 kb and encodes a protein of 662 amino acids with a molecular weight of about 73 kDa. The mouse HIF3α transcript was found to be expressed in thymus, lung, brain, heart, and kidney. Based on the mouse HIF3α sequence, the authors identified a human HIF3α cDNA fragment (Genbank accession number AF079154) and mapped the human HIF3α gene locus on chromosome 19q13.13-q13.2. Sequence similarities of about 57% and 53% of the Nterminal part (the bHLH-PAS region) of HIF3α with HIF1α and HIF2α, respectively, have been described. Sequence analysis gave rise to the suggestion that the N-terminal transactivation domain (NAD, HRD1) is present in HIF3α, but not the C-terminal transactivation domain (CAD, HRD2). Thus, HIF3α shares a high degree of similarity in the N-terminal region with human HIF1α and HIF2α, but not in the C-terminal region. As already described for the other HIF α-class proteins, HIF3α dimerizes with HIF1β (ARNT). Experiments performed with HRE containing reporter genes revealed that HIF3α suppresses hypoxia-inducible gene expression and therefore might be a negative regulator for HIF-mediated gene expression (Hara et al., Biochem. Biophys. Res. Comm. 2001, 287: 808-813). Hara and coworkers further characterized human HIF3α on the basis of a partial human HIF3α cDNA (Genbank accession number AF079154), the EST clone with the accession number AA359276, and the genomic DNA sequence with the Genbank accession number AC007193. The authors showed that the full length HIF3α cDNA, harbouring 15 exons (Genbank accession number AB054067), encodes a protein of 668 amino acids which is 81.9%, 35.9%, and 35.1% identical to mouse HIF3α, human HIF1α, and to human HIF2α, respectively, and wherein a bHLH domain (aa 12-65), a PAS domain (aa 87-338), and a NAD domain (aa 454-506) are present. To date, little is known about the expression and function of HIF3α. Expression of HIF3α was detected in the developing trachea, olfactory epithelium and human kidney. Assumptions about the function of HIF3α are mainly based on existing data for the α-class homologue HIF1α. Because of the different C-terminal structure of HIF3α, the protein exhibits other characteristics in transfection experiments than HIF1α or HIF2α, for instance an unaltered HIF3α level under hypoxic conditions (Hara et al., Biochem. Biophys. Res. Comm. 2001, 287: 808-813). Thus, HIF3α may play a distinct role in mediating responses to hypoxia. Interesting to note is the detection of a splice variant of the HIF3α locus in the mouse by Makino and coworkers. This splice product functions as a dominant negative regulator of HIF in dimerizing with the a-class proteins and was therefore named inhibitory PAS domain protein (IPAS) (Genbank accession number AF481145-AF481147) (Makino et al., J. Biol. Chem. 2002, 277: 32405-32408). IPAS, which is predominantly expressed in Purkinje neurons of the cerebellum and the cornea epithelium, forms complexes with those HIF proteins which fail to bind to the HRE elements of their respective target genes. Further, Makino et al. showed upregulated IPAS mRNA levels due to hypoxia and a corresponding HIF3α mRNA downregulation. Recently, in an attempt to identify orphan HIF-like proteins in the data base, Maynard and coworkers (Maynard et al., J. Biol. Chem. 2003, 278: 11032-11040) found and described multiple splice variants of the human HIF3alpha locus: hHIF-3alpha1 (Genbank accession numbers ,AB054067, NM_{—}152794, AC007193, as already reported earlier by Hara et al., Biochem. Biophys. Res. Comm. 2001, 287: 808-813), hHIF-3alpha2, also referred to as human inhibitory PAS domain protein (hIPAS) (Genbank accession numbers NM_{—}152795, AF463492 and ESTs BG699633, AL528423 and AL519496), hHIF-3alpha3 (Genbank accession numbers NM_{—}022462, AK021653 and EST BQ067192), hHIF-3alpha4 (Genbank accession number BC026308), hHIF-3alpha5 (Genbank accession number NM_{—}152796 and EST AL535689) and hHIF-3alpha6 (Genbank accession number AK024095). According to Makino et al., the human HIF-3alpha gene consists of 19 exons, spans about 43 kb, and the three exons 1a, 1b and 1c contain the transcription start sites for the at least six splice variants identified.

The singular forms “a”, “an”, and “the” as used herein and in the claims include plural reference unless the context dictates otherwise. For example, “a cell” means as well a plurality of cells, and so forth. The term “and/or” as used in the present specification and in the claims implies that the phrases before and after this term are to be considered either as alternatives or in combination. For instance, the wording “determination of a level and/or an activity” means that either only a level, or only an activity, or both a level and an activity are determined. The term “level” as used herein is meant to comprise a gage of, or a measure of the amount of, or a concentration of a transcription product, for instance an mRNA, or a translation product, for instance a protein or polypeptide. The term “activity” as used herein shall be understood as a measure for the ability of a transcription product or a translation product to produce a biological effect or a measure for a level of biologically active molecules. The term “activity” also refers to enzymatic activity or to biological activity and/or pharmacological activity which refers to binding, antagonization, repression, blocking or neutralization. The terms “level” and/or “activity” as used herein further refer to gene expression levels or gene activity. Gene expression can be defined as the utilization of the information contained in a gene by transcription and translation leading to the production of a gene product. “Dysregulation” shall mean an upregulation or downregulation of gene expression. A gene product comprises either RNA or protein and is the result of expression of a gene. The amount of a gene product can be used to measure how active a gene is. The term “gene” as used in the present specification and in the claims comprises both coding regions (exons) as well as non-coding regions (e.g. non-coding regulatory elements such as promoters or enhancers, introns, leader and trailer sequences). The term “ORF” is an acronym for “open reading frame” and refers to a nucleic acid sequence that does not possess a stop codon in at least one reading frame and therefore can potentially be translated into a sequence of amino acids. “Regulatory elements” shall comprise inducible and non-inducible promoters, enhancers, operators, and other elements that drive and regulate gene expression. The term “fragment” as used herein is meant to comprise e.g. an alternatively spliced, or truncated, or otherwise cleaved transcription product or translation product. The term “derivative” as used herein refers to a mutant, or an RNA-edited, or a chemically modified, or otherwise altered transcription product, or to a mutant, or chemically modified, or otherwise altered translation product. For the purpose of clarity, a derivative transcript, for instance, refers to a transcript having alterations in the nucleic acid sequence such as single or multiple nucleotide deletions, insertions, or exchanges. A “derivative” may be generated by processes such as altered phosphorylation, or glycosylation, or acetylation, or lipidation, or by altered signal peptide cleavage or other types of maturation cleavage. These processes may occur post-translationally. The term “modulator” as used in the present invention and in the claims refers to a molecule capable of changing or altering the level and/or the activity of a gene, or a transcription product of a gene, or a translation product of a gene. Preferably, a “modulator” is capable of changing or altering the biological activity of a transcription product or a translation product of a gene. Said modulation, for instance, may be an increase or a decrease in the biological activity and/or pharmacological activity, in enzyme activity, a change in binding characteristics, or any other change or alteration in the biological, functional, or immunological properties of said translation product of a gene. A “modulator” refers to a molecule which has the capacity to either enhance or inhibit, thus to “modulate” a functional property of an ion channel subunit or an ion channel, to “modulate” binding, antagonization, repression, blocking, neutralization or sequestration of an ion channel or ion channel subunit and to “modulate” activation, agonization and upregulation. “Modulation” will be also used to refer to the capacity to affect the biological activity of a cell. The terms “modulator”, “agent”, “reagent”, or “compound” refer to any substance, chemical, composition or extract that have a positive or negative biological effect on a cell, tissue, body fluid, or within the context of any biological system, or any assay system examined. They can be agonists, antagonists, partial agonists or inverse agonists of a target. They may be nucleic acids, natural or synthetic peptides or protein complexes, or fusion proteins. They may also be antibodies, organic or anorganic molecules or compositions, small molecules, drugs and any combinations of any of said agents above. They may be used for testing, for diagnostic or for therapeutic purposes. Such modulators, agents, reagents or compounds can be factors present in cell culture media, or sera used for cell culturing, factors such as trophic factors. “Trophic factors” as used in the present invention include but are not limited to neurotrophic factors, to neuregulins, to cytokines, to neurokines, to neuroimmune factors, to factors derived from the brain (BDNF) and to factors of the TGF beta family. Examples of such trophic factors are neurotrophin 3 (NT-3), neurotrophin 4/5 (NT-4/5), nerve growth factor (NGF), fibroblast growth factor (FGF), epidermal growth factor (EGF), interleukin-beta, glial cell-derived neurotrophic factors (GDNF), ciliary neurotrophic factor (CNTF), insulin-like growth factor (IGF), transforming growth factor (TGF) and platelet-derived growth factor (PDGF). The terms “oligonucleotide primer” or “primer” refer to short nucleic acid sequences which can anneal to a given target polynucleotide by hybridization of the complementary base pairs and can be extended by a polymerase. They may be chosen to be specific to a particular sequence or they may be randomly selected, e.g. they will prime all possible sequences in a mix. The length of primers used herein may vary from 10 nucleotides to 80 nucleotides. “Probes” are short nucleic acid sequences of the nucleic acid sequences described and disclosed herein or sequences complementary therewith. They may comprise full length sequences, or fragments, derivatives, isoforms, or variants of a given sequence. The identification of hybridization complexes between a “probe” and an assayed sample allows the detection of the presence of other similar sequences within that sample. As used herein, “homolog or homology” is a term used in the art to describe the relatedness of a nucleotide or peptide sequence to another nucleotide or peptide sequence, which is determined by the degree of identity and/or similarity between said sequences compared. In the art, the terms “identity” and “similarity” mean the degree of polypeptide or polynucleotide sequence relatedness which are determined by matching a query sequence and other sequences of preferably the same type (nucleic acid or protein sequence) with each other. Preferred computer program methods to calculate and determine “identity” and “similarity” include, but are not limited to GCG BLAST (Basic Local Alignment Search Tool) (Altschul et al., J. Mol. Biol. 1990, 215: 403-410; Altschul et al., Nucleic Acids Res. 1997, 25: 3389-3402; Devereux et al., Nucleic Acids Res. 1984, 12: 387), BLASTN 2.0 (Gish W., http://blast.wustl.edu, 1996-2002), FASTA (Pearson and Lipman, Proc. Natl. Acad. Sci. USA 1988, 85: 2444-2448), and GCG GelMerge which determines and aligns a pair of contigs with the longest overlap (Wilbur and Lipman, SIAM J. Appl. Math. 1984, 44: 557-567; Needleman and Wunsch, J. Mol. Biol. 1970, 48: 443-453). The term “variant” as used herein refers to any polypeptide or protein, in reference to polypeptides and proteins disclosed in the present invention, in which one or more amino acids are added and/or substituted and/or deleted and/or inserted at the N-terminus, and/or the C-terminus, and/or within the native amino acid sequences of the native polypeptides or proteins of the present invention. Furthermore, the term “variant” shall include any shorter or longer version of a polypeptide or protein. “Variants” shall also comprise a sequence that has at least about 80% sequence identity, more preferably at least about 90% sequence identity, and most preferably at least about 95% sequence identity with the amino acid sequences of HIF3a, SEQ ID NO. 2, SEQ ID NO. 3, SEQ ID NO. 4, SEQ ID NO. 5. “Variants” of a protein molecule shown in SEQ ID NO.2, SEQ ID NO. 3, SEQ ID NO. 4 and SEQ ID NO. 5 include, for example, proteins with conservative amino acid substitutions in highly conservative regions. “Proteins and polypeptides” of the present invention include variants, fragments and chemical derivatives of the protein comprising the amino acid sequences of HIF3a, SEQ ID NO. 2, SEQ ID NO. 3, SEQ ID NO. 4, SEQ ID NO. 5. Sequence variations shall be included wherein a codon are replaced with another codon due to alternative base sequences, but the amino acid sequence translated by the DNA sequence remains unchanged. This known in the art phenomenon is called redundancy of the set of codons which translate specific amino acids. Included shall be such exchange of amino acids which would have no effect on functionality, such as arginine for lysine, valine for leucine, asparagine for glutamine. Proteins and polypeptides can be included which can be isolated from nature or be produced by recombinant and/or synthetic means. Native proteins or polypeptides refer to naturally-occurring truncated or secreted forms, naturally occurring variant forms (e.g. splice-variants) and naturally occurring allelic variants. The term “isolated” as used herein is considered to refer to molecules or substances which have been changed and/or that are removed from their natural environment, i.e. isolated from a cell or from a living crganism in which they normally occur, and that are separated or essentially purified from the coexisting components with which they are found to be associated in nature, it is also said that they are “non-native”. This notion further means that the sequences encoding such molecules can be linked by the hand of man to polynucleotides to which they are not linked in their natural state and such molecules can be produced by recombinant and/or synthetic means (non-native). Even if for said purposes those sequences may be introduced into living or non-living organisms by methods known to those skilled in the art, and even if those sequences are still present in said organisms, they are still considered to be isolated, to be non-native. In the present invention, the terms “risk”, “susceptibility”, and “predisposition” are tantamount and are used with respect to the probability of developing a neurodegenerative disease, preferably Alzheimer's disease.

The term “AD” shall mean Alzheimer's disease. “AD-type neuropathology” as used herein refers to neuropathological, neurophysiological, histopathological and clinical hallmarks as described in the instant invention and as commonly known from state-of-the-art literature (see: lqbal, Swaab, Winblad and Wisniewski, Alzheimer's Disease and Related Disorders (Etiology, Pathogenesis and Therapeutics), Wiley & Sons, New York, Weinheim, Toronto, 1999; Scinto and Daffner, Early Diagnosis of Alzheimer's Disease, Humana Press, Totowa, N.J., 2000; Mayeux and Christen, Epidemiology of Alzheimer's Disease: From Gene to Prevention, Springer Press, Berlin, Heidelberg, N.Y., 1999; Younkin, Tanzi and Christen, Presenilins and Alzheimer's Disease, Springer Press, Berlin, Heidelberg, N.Y., 1998). The term “Braak stage” or “Braak staging” refers to the classification of brains according to the criteria proposed by Braak and Braak (Braak and Braak, Acta Neuropathology 1991, 82: 239-259). On the basis of the distribution of neurofibrillary tangles and neuropil threads, the neuropathologic progression of AD is divided into six stages (stage 0 to 6). In the instant invention Braak stages 0 to 2 represent healthy control persons (“controls”), and Braak stages 4 to 6 represent persons suffering from Alzheimer's disease (“AD patients”). The values obtained from said “controls” are the “reference values” representing a “known health status” and the values obtained from said “AD patients” are the “reference values” representing a “known disease status”. Braak stage 3 (middle Braak stage) may represent either a healthy control persons or an AD patient. The higher the Braak stage the more likely is the possibility to display the symptoms of AD. For a neuropathological assessment, i.e. an estimation of the probability that pathological changes of AD are the underlying cause of dementia, a recommendation is given by Braak H. (www.alzforum.org).

Neurodegenerative diseases or disorders according to the present invention comprise Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia and other tauopathies, and mild-cognitive impairment. Conditions involving neurodegenerative processes are, for instance, age-related macular degeneration, narcolepsy, motor neuron diseases, prion diseases and traumatic nerve injury and repair, and multiple sclerosis.

The present invention discloses the identification, differential expression, the differential regulation, a dysregulation of a gene coding for HIF3α-, alias HIF3alpha-, alias HIF3a, in specific samples, in specific brain regions of AD patients and/or in comparison to control persons. The present invention discloses that the gene expression for HIF3a is varied, is dysregulated in AD-affected brains, in that HIF3a mRNA levels are elevated, are up-regulated in the temporal cortex and/or the hippocampus as compared to the frontal cortex, or are down-regulated in the frontal cortex as compared to the temporal cortex and/or the hippocampus. Further, the present invention discloses that the HIF3a expression differs between the frontal cortex and the temporal cortex and/or the hippocampus of healthy age-matched control subjects compared to the frontal cortex and the temporal cortex and/or the hippocampus of AD patients. No such dysregulation is observed in samples obtained from age-matched, healthy controls. HIF3a is elevated in the temporal cortex but not frontal cortex of AD-patients compared to controls. This dysregulation presumably relates to a pathologic alteration of HIF3a signaling in AD-affected brains. To date, no experiments have been described that demonstrate a relationship between the dysregulation of HIF3a gene expression and the pathology of neurodegenerative diseases, in particular AD. Likewise, no mutations in the HIF3a gene have been described to be associated with said diseases. Linking the HIF3a gene to such diseases offers new ways, inter alia, for the diagnosis and treatment of said diseases.

The present invention discloses a dysregulation of a gene coding for HIF3a in specific brain regions of AD patients. Neurons within the inferior temporal lobe, the entorhinal cortex, the hippocampus, and the amygdala are subject to degenerative processes in AD (Terry et al., Annals of Neurology 1981, 10:184-192). These brain regions are mostly involved in the processing of learning and memory functions and display a selective vulnerability to neuronal loss and degeneration in AD. In contrast, neurons within the frontal cortex, the occipital cortex, and the cerebellum remain largely intact and preserved from neurodegenerative processes. Brain tissues from the frontal cortex (F), the temporal cortex (T), and the hippocampus (H) of AD patients and healthy, age-matched control individuals were used for the herein disclosed examples. Consequently, the HIF3a gene and its corresponding transcription and/or translation products have a causative role in the regional selective neuronal degeneration typically observed in AD. Alternatively, HIF3a may confer a neuroprotective function to the remaining surviving nerve cells. Based on these disclosures, the present invention has utility for the diagnostic evaluation and prognosis as well as for the identification of a predisposition to a neurodegenerative disease, in particular AD. Furthermore, the present invention provides methods for the diagnostic monitoring of patients undergoing treatment for such a disease.

In one aspect, the invention features a method of diagnosing or prognosticating a neurodegenerative disease in a subject, or determining whether a subject is at increased risk of developing said disease. The method comprises: determining a level, or an activity, or both said level and said activity of (i) a transcription product of a gene coding for HIF3a, and/or of (ii) a translation product of a gene coding for HIF3a, and/or of (iii) a fragment, or derivative, or variant of said transcription and/or said translation product, in a sample obtained from said subject and comparing said level, and/or said activity of said transcription product and/or said translation product to a reference value representing a known disease status and/or to a reference value representing a known health status, and said level and/or said activity is varied or altered compared to a reference value representing a known health status (control), and/or is similar or equal to a reference value representing a known disease status, preferably a disease status of AD (AD patient), thereby diagnosing or prognosticating said neurodegenerative disease in said subject, or determining whether said subject is at increased risk of developing said neurodegenerative disease. The wording “in a subject” refers to results of the methods disclosed as far as they relate to a disease afflicting a subject, that is to say, said disease being “in” a subject.

The invention also relates to the construction and the use of primers and probes which are unique to the nucleic acid sequences, or fragments, or variants thereof, as disclosed in the present invention. The oligonucleotide primers and/or probes can be labeled specifically with fluorescent, bioluminescent, magnetic, or radioactive substances. The invention further relates to the detection and the production of said nucleic acid sequences, or fragments and variants thereof, using said specific oligonucleotide primers in appropriate combinations. PCR-analysis, a method well known to those skilled in the art, can be performed with said primer combinations to amplify said gene specific nucleic acid sequences from a sample containing nucleic acids. Such sample may be derived either from healthy or diseased subjects. Whether an amplification results in a specific nucleic acid product or not, and whether a fragment of different length can be obtained or not, may be indicative for a neurodegenerative disease, in particular Alzheimer's disease. Thus, the invention provides nucleic acid sequences, oligonucleotide primers, and probes of at least 10 bases in length up to the entire coding and gene sequences, useful for the detection of gene mutations and single nucleotide polymorphisms in a given sample comprising nucleic acid sequences to be examined, which may be associated with neurodegenerative diseases, in particular Alzheimer's disease. This feature has utility for developing rapid DNA-based diagnostic tests, preferably also in the format of a kit. Primers for HIF3a are exemplarily described in Example (viii).

In a further aspect, the invention features a method of monitoring the progression of a neurodegenerative disease in a subject. A level, or an activity, or both said level and said activity, of (i) a transcription product of a gene coding for HIF3a, and/or of (ii) a translation product of a gene coding for HIF3a, and/or of (iii) a fragment, or derivative, or variant of said transcription or translation product in a sample obtained from said subject is determined. Said level and/or said activity is compared to a reference value representing a known disease or health status. Thereby, the progression of said neurodegenerative disease in said subject is monitored.

In still a further aspect, the invention features a method of evaluating a treatment for a neurodegenerative disease, comprising determining a level, or an activity, or both said level and said activity of (i) a transcription product of a gene coding for HIF3a, and/or of (ii) a translation product of a gene coding for HIF3a, and/or of (iii) a fragment, or derivative, or variant of said transcription or translation product in a sample obtained from a subject being treated for said disease. Said level, or said activity, or both said level and said activity are compared to a reference value representing a known disease or health status, thereby evaluating the treatment for said neurodegenerative disease.

In a preferred embodiment of the herein claimed methods, kits, recombinant animals, molecules, assays, and uses of the instant invention, said gene coding for a hypoxia-inducible factor (HIF), is the gene coding for a hypoxia-inducible factor 3 alpha protein, also termed HIF3 α, HIF-3 alpha, HIF3 alpha or simply HIF3a. The gene HIF3a is also referred to as the splice variant (sv) HIF3a sv1, represented by the cDNA sequence of SEQ ID NO. 6 (Genbank accession number AK021421, sequence corrected on the basis of EST and mRNA sequence information from the Genbank data base, refer to FIG. 12), and also referred to as the splice variant HIF3a sv2, represented by the CDNA sequence of SEQ ID NO. 7 (Genbank accession number BC026308, sequence corrected on the basis of EST and mRNA sequence information from the Genbank data base, refer to FIG. 13), and also referred to as the splice variant HIF3a sv3, represented by the cDNA sequence of SEQ ID NO. 8 (Genbank accession number AK027725, sequence corrected on the basis of EST and mRNA sequence information from the Genbank data base, refer to FIG. 14), and also referred to as the splice variant HIF3a sv5, represented by the cDNA sequence of SEQ ID NO. 9 (Genbank accession number AK021653, sequence corrected on the basis of EST and mRNA sequence information from the Genbank data base, refer to FIG. 15). In the instant invention said sequences are “isolated” as the term is employed herein. Further, in the instant invention, the gene coding for HIF3a protein and all splice variants as disclosed, is also generally referred to as the HIF3a gene, or simply HIF3a.

In another preferred embodiment of the herein claimed methods, kits, recombinant animals, molecules, assays, and uses of the instant invention, said hypoxia-inducible factor (HIF) protein, is the hypoxia-inducible factor 3 alpha protein, also termed HIF3 α, HIF-3 alpha, HIF3 alpha, or HIF3a. The protein HIF3a is also referred to as the HIF3a splice variant 1 (sv1) protein, represented by SEQ ID NO. 2 (FIG. 8) and by the coding sequence of HIF3a sv1(SEQ ID NO. 10, FIG. 16), and also referred to the HIF3a protein HIF3a splice variant 2 (sv2), represented by SEQ ID NO. 3 (FIG. 9) and by the coding sequence of HIF3a sv2 (SEQ ID NO. 11, FIG. 17), and also referred to the HIF3a protein HIF3a splice variant 3 (sv3), represented by SEQ ID NO. 4 (FIG. 10) and by the coding sequence of HIF3a sv3 (SEQ ID NO. 12, FIG. 18), and also referred to the HIF3a protein HIF3a splice variant 5 (sv5), represented by SEQ ID NO. 5, which is similar to protein BAB13865.1 of the Genbank data base (FIG. 11) and by the coding sequence of HIF3a sv5 (SEQ ID NO. 13, FIG. 19). In the instant invention, said sequences are “isolated” as the term is employed herein. Further, in the instant invention, said HIF3a proteins encoded by the HIF3a gene, HIF3a sv1, HIF3a sv2, HIF3a sv3, HIF3a sv5, are also generally referred to as the HIF3a proteins, or simply HIF3a.

In a further preferred embodiment of the herein claimed methods, kits, recombinant animals, molecules, assays, and uses of the instant invention, said neurodegenerative disease or disorder is Alzheimer's disease, and said subjects suffer from Alzheimer's disease.

It is preferred that the sample to be analyzed and determined is selected from the group comprising brain tissue or other tissues, or other body cells. The sample can also comprise cerebrospinal fluid or other body fluids including saliva, urine, serum plasma, blood, or mucus. Preferably, the methods of diagnosis, prognosis, monitoring the progression or evaluating a treatment for a neurodegenerative disease, according to the instant invention, can be practiced ex corpore, and such methods preferably relate to samples, for instance, body fluids or cells, removed, collected, or isolated from a subject or patient or healthy control person.

In further preferred embodiments, said reference value is that of a level, or an activity, or both said level and said activity of (i) a transcription product of a gene coding for HIF3a, and/or of (ii) a translation product of a gene coding for HIF3a, and/or of (iii) a fragment, or derivative, or variant of said transcription or translation product in a sample obtained from a subject not suffering from said neurodegenerative disease (healthy control person, control sample, control) or in a sample obtained from a subject suffering from a neurodegenerative disease, in particular Alzheimer's disease (patient sample, patient).

In preferred embodiments, an alteration in the level and/or activity, a varied level and/or activity of a transcription product of a gene coding for HIF3a and/or of a translation product of a gene coding for HIF3a and/or of a fragment, or derivative, or variant thereof in a sample cell, or tissue, or body fluid from said subject relative to a reference value representing a known health status (control sample) indicates a diagnosis, or prognosis, or increased risk of becoming diseased with a neurodegenerative disease, particularly AD.

In further preferred embodiments, an equal or similar level and/or activity of a transcription product of the gene coding for a HIF3a protein and/or of a translation product of the gene coding for a HIF3a protein and/or of a fragment, or derivative, or variant thereof in a sample cell, or tissue, or body fluid obtained from a subject relative to a reference value representing a known disease status of a neurodegenerative disease, in particular Alzheimer's disease (AD patient sample), indicates a diagnosis, or prognosis, or increased risk of becoming diseased with said neurodegenerative disease.

In preferred embodiments, measurement of the level of transcription products of an HIF3a gene is performed in a sample obtained from a subject using a quantitative PCR-analysis with primer combinations to amplify said gene specific sequences from cDNA obtained by reverse transcription of RNA extracted from a sample of a subject. Primer combinations are given in Example (viii) of the instant invention, but also other primers generated from the sequences as disclosed in the instant invention can be used. A Northern blot with probes specific for said gene can also be applied. It might further be preferred to measure transcription products by means of chip-based microarray technologies. These techniques are known to those of ordinary skill in the art (see e.g. Sambrook and Russell, Molecular Cloning: A Laboratory Manual, Cold Spring Harbor Laboratory Press, Cold Spring Harbor, N.Y., 2001; Schena M., Microarray Biochip Technology, Eaton Publishing, Natick, Mass., 2000). An example of an immunoassay is the detection and measurement of enzyme activity as disclosed and described in the patent application WO 02/14543.

Furthermore, a level and/or activity of a translation product of a gene coding for HIF3a and/or of a fragment, or derivative, or variant of said translation product, and/or the level of activity of said translation product, and/or of a fragment, or derivative, or variant thereof, can be detected using an immunoassay, an activity assay, and/or a binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots, and other techniques known to those of ordinary skill in the art (see Harlow and Lane, Using Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). All these detection techniques may also be employed in the format of microarrays, protein-arrays, antibody microarrays, tissue microarrays, electronic biochip or protein-chip based technologies (see Schena M., Microarray Biochip Technology, Eaton Publishing, Natick, Mass., 2000).

In a preferred embodiment, the level, or the activity, or both said level and said activity of (i) a transcription product of a gene coding for HIF3a, and/or of (ii) a translation product of a gene coding for HIF3a, and/or of (iii) a fragment, or derivative, or variant of said transcription or translation product in a series of samples taken from said subject over a period of time is compared, in order to monitor the progression of said disease. In further preferred embodiments, said subject receives a treatment prior to one or more of said sample gatherings. In yet another preferred embodiment, said level and/or activity is determined before and after said treatment of s aid subject.

In another aspect, the invention features a kit for diagnosing or prognosticating neurodegenerative diseases, in particular AD, in a subject, or determining the propensity or predisposition of a subject, the risk of a subject to develop a neurodegenerative disease, in particular AD, said kit comprising:
(a) at least one reagent which is selected from the group consisting of (i) reagents that selectively detect a transcription product of a gene coding for HIF3a, and (ii) reagents that selectively detect a translation product of a gene coding for HIF3a; and
(b) an instruction for diagnosing, or prognosticating a neurodegenerative disease, in particular AD, or determining the propensity or predisposition of a subject to develop such a disease by
   detecting a level, or an activity, or both said level and said activity, of said transcription product and/or said translation product of a gene coding for HIF3a, in a sample obtained from said subject; and
   diagnosing or prognosticating a neurodegenerative disease, in particular AD, or determining the propensity or predisposition, the risk of said subject to develop such a disease, wherein a varied or altered level, or activity, or both said level and said activity, of said transcription product and/or said translation product compared to a reference value representing a known health status (control), and/or wherein a level, or activity, or both said level and said activity, of said transcription product and/or said translation product is similar or equal to a reference value representing a known disease status, preferably a disease status of AD, indicates a diagnosis or prognosis of a neurodegenerative disease, in particular AD, or an increased propensity or predisposition of developing such a disease. The kit, according to the present invention, may be particularly useful for the identification of individuals that are at risk of developing a neurodegenerative disease, in particular AD.

In a further aspect the invention features the use of a kit in a method of diagnosing or prognosticating a neurodegenerative disease, in particular Alzheimer's disease, in a subject, and in a method of determining the propensity or predisposition of a subject to develop such a disease by the steps of: (i) detecting in a sample obtained from said subject a level, or an activity, or both said level and said activity of a transcription product and/or of a translation product of a gene coding for HIF3a, and (ii) comparing said level or activity, or both said level and said activity of a transcription product and/or of a translation product of a gene coding for HIF3a to a reference value representing a known health status and/or to a reference value representing a known disease status, and said level, or activity, or both said level and said activity, of said transcription product and/or said translation product is varied compared to a reference value representing a known health status, and/or is similar or equal to a reference value representing a known disease status.

Consequently, the kit, according to the invention, may serve as a means for targeting identified individuals for early preventive measures or therapeutic intervention prior to disease onset, before irreversible damage in the course of the disease has been inflicted. Furthermore, in preferred embodiments, the kit featured in the invention is useful for monitoring a progression of a neurodegenerative disease, in particular AD, in a subject, as well as monitoring success or failure of therapeutic treatment for such a disease of said subject.

In another aspect, the invention features a method of treating or preventing a neurodegenerative disease, in particular AD, in a subject comprising the administration to said subject in a therapeutically or prophylactically effective amount of an agent or agents which directly or indirectly affect a level, or an activity, or both said level and said activity, of (i) a gene coding for HIF3a, and/or (ii) a transcription product of a gene coding for HIF3a, and/or (iii) a translation product of a gene coding for HIF3a and/or (iv) a fragment, or derivative, or variant of (i) to (iii). Said agent may comprise a small molecule, or it may also comprise a peptide, an oligopeptide, or a polypeptide. Said peptide, oligopeptide, or polypeptide may comprise an amino acid sequence of a translation product of a gene coding for HIF3a protein, or a fragment, or derivative, or a variant thereof. An agent for treating or preventing a neurodegenerative disease, in particular AD, according to the instant invention, may also consist of a nucleotide, an oligonucleotide, or a polynucleotide. Said oligonucleotide or polynucleotide may comprise a nucleotide sequence of a gene coding for HIF3a protein, either in sense orientation or in antisense orientation.

In preferred embodiments, the method comprises the application of per se known methods of gene therapy and/or antisense nucleic acid technology to administer said agent or agents. In general, gene therapy includes several approaches: molecular replacement of a mutated gene, addition of a new gene resulting in the synthesis of a therapeutic protein, and modulation of endogenous cellular gene expression by recombinant expression methods or by drugs. Gene-transfer techniques are described in detail (see e.g. Behr, Acc Chem Res 1993, 26: 274-278 and Mulligan, Science 1993, 260: 926-931) and include direct gene-transfer techniques such as mechanical microinjection of DNA into a cell as well as indirect techniques employing biological vectors (like recombinant viruses, especially retroviruses) or model liposomes, or techniques based on transfection with DNA coprecipitation with polycations, cell membrane pertubation by chemical (solvents, detergents, polymers, enzymes) or physical means (mechanic, osmotic, thermic, electric shocks). The postnatal gene transfer into the central nervous system has been described in detail (see e.g. Wolff, Curr Opin Neurobiol 1993, 3: 743-748).

In particular, the invention features a method of treating or preventing a neurodegenerative disease by means of antisense nucleic acid therapy, i.e. the down-regulation of an inappropriately expressed or defective gene by the introduction of antisense nucleic acids or derivatives thereof into certain critical cells (see e.g. Gillespie, DN&P 1992, 5: 389-395; Agrawal and.Akhtar, Trends Biotechnol 1995, 13: 197-199; Crooke, Biotechnology 1992, 10: 882-6). Apart from hybridization strategies, the application of ribozymes, i.e. RNA molecules that act as enzymes, destroying RNA that carries the message of disease has also been described (see e.g. Barinaga, Science 1993, 262: 1512-1514). In preferred embodiments, the subject to be treated is a human, and therapeutic antisense nucleic acids or derivatives thereof are directed against transcripts of a gene coding for HIF3a. It is preferred that cells of the central nervous system, preferably the brain, of a subject are treated in such a way. Cell penetration can be performed by known strategies such as coupling of antisense nucleic acids and derivatives thereof to carrier particles, or the above described techniques. Strategies for administering targeted therapeutic oligodeoxynucleotides are known to those of skill in the art (see e.g. Wickstrom, Trends Biotechnol 1992, 10: 281-287). In some cases, delivery can be performed by mere topical application. Further approaches are directed to intracellular expression of antisense RNA. In this strategy, cells are transformed ex vivo with a recombinant gene that directs the synthesis of an RNA that is complementary to a region of target nucleic acid. Therapeutical use of intracellularly expressed antisense RNA is procedurally similar to gene therapy. A recently developed method of regulating the intracellular expression of genes by the use of double-stranded RNA, known variously as RNA interference (RNAi), can be another effective approach for nucleic acid therapy (Hannon, Nature 2002, 418: 244-251).

In further preferred embodiments, the method comprises grafting donor cells into the central nervous system, preferably the brain, of said subject, or donor cells preferably treated so as to minimize or reduce graft rejection, wherein said donor cells are genetically modified by insertion of at least one transgene encoding said agent or agents. Said transgene might be carried by a viral vector, in particular a retroviral vector. The transgene can be inserted into the donor cells by a nonviral physical transfection of DNA encoding a transgene, in particular by microinjection. Insertion of the transgene can also be performed by electroporation, chemically mediated transfection, in particular calcium phosphate transfection or liposomal mediated transfection (see Mc Celland and Pardee, Expression Genetics: Accelerated and High-Throughput Methods, Eaton Publishing, Natick, Mass., 1999).

In preferred embodiments, said agent for treating and preventing a neurodegenerative disease, in particular AD, is a therapeutic protein which can be administered to said subject, preferably a human, by a process comprising introducing subject cells into said subject, said subject cells having been treated in vitro to insert a DNA segment encoding said therapeutic protein, said subject cells expressing in vivo in said subject a therapeutically effective amount of said therapeutic protein. Said DNA segment can be inserted into said cells in vitro by a viral vector, in particular a retroviral vector.

Methods of treatment, according to the present invention, comprise the application of therapeutic cloning, transplantation, and stem cell therapy using embryonic stem cells or embryonic germ cells and neuronal adult stem cells, combined with any of the previously described cell- and gene therapeutic methods. Stem cells may be totipotent or pluripotent. They may also be organ-specific. Strategies for repairing diseased and/or damaged brain cells or tissue comprise (i) taking donor cells from an adult tissue. Nuclei of those cells are transplanted into unfertilized egg cells from which the genetic material has been removed. Embryonic stem cells are isolated from the blastocyst stage of the cells which underwent somatic cell nuclear transfer. Use of differentiation factors then leads to a directed development of the stem cells to specialized cell types, preferably neuronal cells (Lanza et al., Nature Medicine 1999, 9: 975-977), or (ii) purifying adult stem cells, isolated from the central nervous system, or from bone marrow (mesenchymal stem cells), for in vitro expansion and subsequent grafting and transplantation, or (iii) directly inducing endogenous neural stem cells to proliferate, migrate, and differentiate into functional neurons (Peterson D A, Curr. Opin. Pharmacol. 2002, 2: 34-42). Adult neural stem cells are of great potential for repairing damaged or diseased brain tissues, as the germinal centers of the adult brain are free of neuronal damage or dysfunction (Colman A, Drug Discovery World 2001, 7: 66-71).

In preferred embodiments, the subject for treatment or prevention, according to the present invention, can be a human, an experimental animal, e.g. a mouse or a rat or a fly, a domestic animal, or a non-human primate. The experimental animal can be a non-human animal model for a neurodegenerative disorder, a genetically altered animal, e.g. a transgenic mouse or fly and/or a knockout mouse or fly preferably displaying symptoms of AD, showing an AD-type neuropathology.

In a further aspect, the invention features a modulator of an activity, or a level, or both said activity and said level of at least one substance which is selected from the group consisting of (i) a gene coding for HIF3a, and/or (ii) a transcription product of a gene coding for HIF3a and/or (iii) a translation product of a gene coding for HIF3a, and/or (iv) a fragment, or derivative, or variant of (i) to (iii).

In an additional aspect, the invention features a pharmaceutical composition comprising said modulator and preferably a pharmaceutical carrier. Said carrier refers to a diluent, adjuvant, excipient, or vehicle with which the modulator is administered.

In a further aspect, the invention features a modulator of an activity, or a level, or both said activity and said level of at least one substance which is selected from the group consisting of (i) a gene coding for HIF3a, and/or (ii) a transcription product of a gene coding for HIF3a, and/or (iii) a translation product of a gene coding for HIF3a, and/or (iv) a fragment, or derivative, or variant of (i) to (iii) for use in a pharmaceutical composition.

In another aspect, the invention provides for the use of a modulator of an activity, or a level, or both said activity and said level of at least one substance which is selected from the group consisting of (i) a gene coding for HIF3a, and/or (ii) a transcription product of a gene coding for HIF3a and/or (iii) a translation product of a gene coding for HIF3a, and/or (iv) a fragment, or derivative, or variant of (i) to (iii) for a preparation of a medicament for treating or preventing a neurodegenerative disease, in particular AD.

In one aspect, the present invention also provides a kit comprising one or more containers filled with a therapeutically or prophylactically effective amount of said pharmaceutical composition.

In another aspect, the present invention features the use of non-native nucleic acid molecules and/or of translation products, protein molecules of the gene coding for human and/or mouse HIF3a and/or fragments, or derivatives, or variants thereof, of nucleic acid molecules as shown in SEQ ID NO. 6, SEQ ID NO. 7, SEQ ID NO. 8, SEQ ID NO. 9, SEQ ID NO. 10, SEQ ID NO. 11, SEQ ID NO.12, SEQ ID NO. 13, and protein molecules as shown in SEQ ID NO. 2, SEQ ID NO. 3, SEQ ID NO. 4, SEQ ID NO. 5, as targeting molecules to generate recombinant, genetically altered non-human animals which are transgenic animals and/or knockout animals. It is preferred that said genetically altered non-human animal is a mammal, preferably a rodent, more preferably a mouse or a rat or a guinea pig. It is further preferred that said genetically altered non-human animal is an invertebrate animal, preferably an insect, more preferably a fly such as the fly Drosophila melanogaster. Further, said genetically altered non-human animal may be a domestic animal, or a non-human primate. In one embodiment, the expression of said genetic alteration results in a non-human animal exhibiting a predisposition to developing symptoms and/or displaying symptoms of neuropathology similar to a neurodegenerative disease, in particular symptoms of a neuropathology similar to AD (AD-type neuropathology), including, inter alia , histological features of AD and behavioural changes characteristic of AD. In another embodiment, the expression of said genetic alteration results in a non-human animal which has a reduced risk of developing symptoms similar to a neurodegenerative disease, in particular a reduced risk of developing symptoms of a neuropathology similar to AD and/or which shows a reduction of AD symptoms and/or which has no AD symptoms due to a beneficial effect caused by the expression of the gene used to genetically alter said non-human animal.

In one aspect, the invention features a recombinant, genetically altered non-human animal comprising a non-native gene sequence coding for HIF3a, or a fragment or a derivative, or a variant thereof, as shown in SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13 and as shown in SEQ ID NO. 2, SEQ ID NO. 3, SEQ ID NO. 4, SEQ ID NO. 5. Said non-native gene sequence coding for HIF3a may be either the human and/or the mouse HIF3a gene sequence. The generation of said recombinant, genetically altered non-human animal comprises (i) the use of non-native nucleic acid molecules and of translation products, protein molecules of the gene coding for human and/or mouse HIF3a and/or fragments, or derivatives, or variants thereof, as shown in SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13 and as shown in SEQ ID NO. 2, SEQ ID NO. 3, SEQ ID NO. 4, SEQ ID NO. 5, for generating a gene targeting construct and (ii) providing said gene targeting construct containing a gene sequence of human and/or mouse HIF3a, or a fragment, or a variant of said gene sequence, and a selectable marker sequence, and (iii) introducing said targeting construct into a stem cell, into an embryonal stem (ES) cell of a non-human animal, and (iv) introducing said non-human animal stem cell into a non-human embryo, and (v) transplanting said embryo into a pseudopregnant non-human animal, and (vi) allowing said embryo to develop to term, and (vii) identifying a genetically altered non-human animal whose genome comprises a modification of said gene sequence in one or both alleles, and (viii) breeding the genetically altered non-human animal of step (vii) to obtain a genetically altered non-human animal whose genome comprises a modification of said endogenous gene. It is preferred that said genetically altered non-human animal expresses a recombinant, an altered gene wherein said expression is a mis-expression, or under-expression, or over-expression, or non-expression. Examples of such targeting constructs containing a gene sequence of human and/or mouse HIF3a and a selectable marker sequence, as well as the expression of said recombinant, altered HIF3a genes in non-human genetically altered animals, preferably animals such as mouse or fly, are disclosed in the present invention (see Example (xii) and FIGS. 39 to 44).

In one preferred embodiment, said gene disruption or suppression or activation or the expression of said genetic alteration results in said non-human animal exhibiting a predisposition to developing symptoms, and/or displaying symptoms of neuropathology similar to a neurodegenerative disease, in particular symptoms of a neuropathology similar to AD (AD-type neuropathology).

In another preferred embodiment, the expression of said genetic alteration results in a non-human animal which has a reduced risk of developing symptoms similar to a neurodegenerative disease, in particular a reduced risk of developing symptoms similar to AD and/or which shows a reduction of AD symptoms and/or which has no AD symptoms due to an effect, which can be a beneficial effect, caused by the expression of the gene used to genetically alter said non-human animal.

In a further preferred embodiment of the present invention, said genetically altered non-human animal is a mammal, preferably a rodent, more preferably a mouse or a rat or a guinea pig. It is further preferred that said genetically altered non-human animal is an invertebrate animal, preferably an insect, more preferably a fly such as the fly Drosophila melanogaster. Further, said genetically altered non-human animal may be a domestic animal, or a non-human primate. Said genetically altered non-human is a transgenic animal and/or a knockout animal.

In a further preferred embodiment of the present invention, said recombinant, genetically altered non-human animal whose genome comprises a non-native gene sequence coding for either the human and/or the mouse HIF3a, or a fragment or a derivative, or a variant thereof, which is generated by the steps of (i)-(viii) and as described in the present invention, is crossed to an Alzheimer's disease animal model as commonly known in the art to produce a transgenic HIF3a animal and/or HIF3a knock-out animal on an Alzheimer's disease background. The impact of HIF3a expression on Alzheimer's disease pathology in said genetically altered non-human transgenic HIF3a animal and/or HIF3a knock-out animal is defined by histological analyses, immunohistochemistry and/or quantification of diffuse and mature plaques in the brain, by staining for certain cell populations and/or for signs of inflammation and neurodegeneration and further, by biochemical analyses like differential extraction of Abeta and/or phosphorylation status of Tau protein. The neurological function is assessed by a battery of behavioural tests including but are not limited to minineurological examinations, rotarod, grip test, hotplate test, zero maze, openfield test, Y maze, Morris water maze and/or active avoidance test. Further, the phenotype of said non-human transgenic HIF3a animal and/or HIF3a knock-out animal is analyzed using gene expression analyses, protein detection methods and histopathology of a variety of organs.

In further preferred embodiment Alzheimer's disease animal models which are used for the crossing with transgenic HIF3a animals and/or HIF3a knock-out animals are selected from genetically altered mice and/or flies expressing human Alzheimer Precursor Protein (APP) and/or mutant forms of APP, e.g. APP with the swedish mutation, and/or human Presenilin-1 or -2 with known mutations as described in the literature (Janus and Westaway, Physiology Behavior 2001, 873-886; Richards et al., J. Neuroscience 2003, 23:8989-9003) and/or human Tau with known mutations, e.g. the P301L mutation (Gotz et al., J. Biological Chemistry 2001, 276:529-534) or double or triple transgenic animals from those or other mouse mutants developing Alzheimer-like pathologies. Further, genetically altered non-human animals are selected such as human APP (hAPP) and Drosophila Presenilin transgenic flies, as for example the UAS-APP69511 and the UAS-DPsn-mutants (L235P), such as UAS-bovine TAU transgenic flies, actin-GAL4 flies and/or gmr-GAL4 flies. Other Alzheimer's disease animal models can be recombinant animal models which are capable of producing neurofibrillary tangles and/or amyloid plaques; recombinant animal models which express a recombinant gene coding for a tau protein, such as human or mouse tau or tau isoforms as the four-repeat isoform or the P301 L mutant tau; recombinant animal models which express a recombinant gene coding for an amyloid precursor protein or a mutant amyloid precursor protein, or beta-amyloid; recombinant animal models which express a recombinant gene coding for a secretase, gamma-secretase, beta-secretase or alpha-secretase, Presenilin1 or Presenilin2; and any recombinant animal models which express a combination of the recombinant genes as described above.

In preferred embodiment of the present invention, said crossing results in HIF3a knockout animals and/or transgenic HIF3a animals on an Alzheimer's disease background which feature a strengthened and boosted predisposition to develop symptoms and/or to display symptoms of neuropathology similar to a neurodegenerative disease, in particular symptoms of a neuropathology similar to AD (AD-type neuropathology), including inter alia histological features of AD and behavioural changes characteristic of AD.

In another preferred embodiment of the present invention, said crossing results in HIF3a knockout animals and/or transgenic HIF3a animals on an Alzheimer's disease background which have a reduction of AD symptoms and/or a reduced risk of developing symptoms similar to a neurodegenerative disease, in particular a reduced risk of developing symptoms of a neuropathology similar to AD, or showing no AD symptoms due to a beneficial effect caused by the expression of the gene used to genetically alter said non-human animal.

The genetically altered non-human transgenic animal and/or a knockout animal can be used as an experimental animal, as a test animal, as an animal model for a neurodegenerative disorder, preferably as an animal model for Alzheimer.

Examples of such genetically altered transgenic non-human animals showing such neuropathological features and/or showing reduced symptoms are disclosed in the present invention (see Examples (xii) and FIGS. 39 to 44).

Strategies and techniques for the generation and construction of such a transgenic and/or knockout animal are known to those of ordinary skill in the art (see e.g. Capecchi, Science 1989, 244: 1288-1292 and Hogan et al., Manipulating the Mouse Embryo: A Laboratory Manual, Cold Spring Harbor Laboratory Press, Cold Spring Harbor, N.Y., 1994 and Jackson and Abbott, Mouse Genetics and Transgenics: A Practical Approach, Oxford University Press, Oxford, England, 1999) and are described in detail in the present invention (see Example (xii)).

In a further aspect of the present invention, it is preferred to make use of such a recombinant, genetically altered non-human animal, transgenic or knockout animal, as an animal model for investigating neurodegenerative diseases, in particular Alzheimer's disease. Such an animal may be a test animal or an experimental animal useful for screening, testing and validating compounds, agents and modulators in the development of diagnostics and therapeutics to treat neurodegenerative diseases, in particular Alzheimer's disease.

In one further aspect, the invention features a screening assay for a modulator of neurodegenerative diseases, in particular AD, or related diseases and disorders of one or more substances selected from the group consisting of (i) a gene coding for HIF3a, and/or (ii) a transcription product of a gene coding for HIF3a, and/or (iii) a translation product of a gene coding for HIF3a, and/or (iv) a fragment, or derivative, or variant of (i) to (iii), comprising (a) administering a test compound to a test animal or experimental animal or animal model, which is predisposed to developing or has already developed symptoms of a neurodegenerative disease or related diseases or disorders, and (b) measuring the activity and/or level of one or more substances recited in (i) to (iv), and (c) measuring the activity and/or level of said substances in a matched control animal which is equally predisposed to developing or has already developed symptoms of said diseases and to which animal no such test compound has been administered, and (d) comparing the activity and/or level of the substance in the animals of step (b) and (c), wherein an alteration in the activity and/or level of substances in the test animal, or experimental animal, or animal model indicates that the test compound is a modulator of said diseases and disorders.

In a preferred embodiment, said test animal, or experimental animal, or animal model and/or said control animal is a recombinant, genetically altered non-human animal which expresses a gene coding for HIF3a, or a fragment, or a derivative, or a variant thereof, under the control of a transcriptional regulatory element which is not the native HIF3a gene transcriptional control regulatory element, as disclosed in the present invention (see below).

In a further aspect, the genetically altered non-human animals according to the present invention provide an in-vivo assay to determine or validate the efficacy of therapies, or modulatory agents, or compounds for the treatment of neurodegenerative diseases, in particular Alzheimer's disease.

In another aspect, the invention features an assay for screening for a modulator, or an agent, or compound of neurodegenerative diseases, in particular AD, or related diseases and disorders of one or more substances selected from the group consisting of (i) a gene coding for HIF3a , and/or (ii) a transcription product of a gene coding for HIF3a, and/or (iii) a translation product of a gene coding for HIF3a, and/or (iv) a fragment, or derivative, or variant of (i) to (iii). This screening method comprises (a) contacting a cell with a test compound, agent, or modulator and (b) measuring the activity, or the level, or both the activity and the level of one or more substances recited in (i) to (iv), and (c) measuring the activity, or the level, or both the activity and the level of said substances in a control cell not contacted with said test compound, and (d) comparing the levels of the substance in the cells of step (b) and (c), wherein an alteration in the activity and/or level of said substances in the contacted cells, or the contacted cells, indicates that the test compound, or agent, or modulator, is a modulator of said diseases and disorders, wherein said modulator can be the activity, or the level, or both the activity and the level of one or more substances recited in (i) to (iv).

Examples of cells used in said screening assay, such as cells over-expressing the HIF3a protein, preferably stably over-expressing the HIF3a sv3 protein, as disclosed in the present invention, are given below (Example (x) and FIG. 35). The examples of the genetically altered animals and cells and screening assays as disclosed, are illustrative only and not intended to limit the remainder of the disclosure in any way.

In another embodiment, the present invention provides a method for producing a medicament comprising the steps of (i) identifying a modulator of neurodegenerative diseases by a method of the aforementioned screening assays and (ii) admixing the modulator, with a pharmaceutical carrier. However, said modulator may also be identifiable by other types of screening assays.

In another aspect, the present invention provides for an assay for testing a compound, preferably for screening a plurality of compounds, for inhibition of binding between a ligand and a translation product of a gene coding for HIF3a, or a fragment, or derivative, or variant thereof. Said screening assay comprises the steps of (i) adding a liquid suspension of said HIF3a translation product, or a fragment, or derivative, or variant thereof, to a plurality of containers, and (ii) adding a compound or a plurality of compounds to be screened for said inhibition to said plurality of containers, and (iii) adding a detectable, preferably a fluorescently labelled ligand to said containers, and (iv) incubating said HIF3a translation product, or said fragment, or derivative, or variant thereof, and said compound or plurality of compounds, and said detectable, preferably fluorescently labelled ligand, and (v) measuring the amounts of preferably the fluorescence associated with said HIF3a translation product, or with said fragment, or derivative, or variant thereof, and (vi) determining the degree of inhibition by one or more of said compounds of binding of said ligand to said HIF3a translation product, or said fragment, or derivative, or variant thereof. Instead of utilizing a fluorescently labelled ligand, it might in some aspects be preferred to use any other detectable label known to the person skilled in the art, e.g. radioactive labels, and detect it accordingly. Said method may be useful for the identification of novel compounds as well as for evaluating compounds which have been improved or otherwise optimized in their ability to inhibit the binding of a ligand to a gene product of the gene coding for HIF3a, or a fragment, or derivative, or variant thereof. One example of a fluorescent binding assay, in this case based on the use of carrier particles, is disclosed and described in patent application WO 00/52451. A further example is the competitive assay method as described in patent WO 02/01226. Preferred signal detection methods for the screening assays of the instant invention are described in the following patent applications: WO 96/13744, WO 98/16814, WO 98/23942, WO 99/17086, WO 99/34195, WO 00/66985, WO 01/59436, WO 01/59416.

In one further embodiment, the present invention provides a method for producing a medicament comprising the steps of (i) identifying a compound as an inhibitor of binding between a ligand and a gene product of a gene coding for HIF3a by the aforementioned inhibitory binding assay and (ii) admixing the compound with a pharmaceutical carrier. However, said compound may also be identifiable by other a types of screening assays.

In another aspect, the invention features an assay for testing a compound, preferably for screening a plurality of compounds to determine the degree of binding of said compounds to a translation product of a gene coding for HIF3a, or to a fragment, or derivative, or variant thereof. Said screening assay comprises (i) adding a liquid suspension of said HIF3a translation product, or a fragment, or derivative, or variant thereof, to a plurality of containers, and (ii) adding a detectable, preferably a fluorescently labelled compound or a plurality of detectable, preferably fluorescently labelled compounds to be screened for said binding to said plurality of containers, and (iii) incubating said HIF3a translation product, or said fragment, or derivative, or variant thereof, and said detectable, preferably fluorescently labelled compound or detectable, preferably fluorescently labelled compounds, and (iv) measuring the amounts of preferably the fluorescence associated with said HIF3a translation product, or with said fragment, or derivative, or variant thereof, and (v) determining the degree of binding by one or more of said compounds to said HIF3a translation product, or said fragment, or derivative, or variant thereof. In this type of assay it might be preferred to use a fluorescent label. However, any other type of detectable label might also be employed. Said assay methods may be useful for the identification of novel compounds as well as for evaluating compounds which have been improved or otherwise optimized in their ability to bind to an HIF3a translation product, or fragment, or derivative, or variant thereof.

In one further embodiment, the present invention provides a method for producing a medicament comprising the steps of (i) identifying a compound as a binder to a gene product of the HIF3a gene by the aforementioned binding assays and (ii) admixing the compound with a pharmaceutical carrier. However, said compound may also be identifiable by other types of screening assays.

In another embodiment, the present invention provides for a medicament obtainable by any of the methods according to the herein claimed screening assays. In one further embodiment, the instant invention provides for a medicament obtained by any of the methods according to the herein claimed screening assays.

The present invention features protein molecules and the use of said protein molecules as shown in SEQ ID NO. 2, SEQ ID NO. 3, SEQ ID NO. 4, SEQ ID NO. 5, said protein molecules being translation products of the gene coding for HIF3a, or fragmenst, or derivatives, or variants thereof, as a diagnostic targets for detecting a neurodegenerative disease, preferably Alzheimer's disease.

The present invention further features protein molecules and the use of said protein molecules as shown in SEQ ID NO. 2, SEQ ID NO. 3, SEQ ID NO. 4, SEQ ID NO. 5, said protein molecules being translation products of the gene coding for HIF3a, or fragments, or derivatives, or variants thereof, as a screening targets for reagents or compounds preventing, or treating, or ameliorating a neurodegenerative disease, preferably Alzheimer's disease.

The present invention features an antibody which is specifically immunoreactive with an immunogen, wherein said immunogen is a translation product of a gene coding for HIF3a, SEQ ID NO. 2, or SEQ ID NO. 3, or SEQ ID NO. 4, or SEQ ID NO. 5, or a fragment, or variant, or derivative thereof. The immunogen may comprise immunogenic or antigenic epitopes or portions of a translation product of said gene, wherein said immunogenic or antigenic portion of a translation product is a polypeptide, and wherein said polypeptide elicits an antibody response in an animal, and wherein said polypeptide is immunospecifically bound by said antibody. Methods for generating antibodies are well known in the art (see Harlow et al., Antibodies, A Laboratory Manual, Cold Spring Harbor Laboratory Press, Cold Spring Harbor, N.Y., 1988). The term “antibody”, as employed in the present invention, encompasses all forms of antibodies known in the art, such as polyclonal, monoclonal, chimeric, recombinatorial, anti-idiotypic, humanized, or single chain antibodies, as well as fragments thereof (see Dubel and Breitling, Recombinant Antibodies, Wiley-Liss, N.Y., NY, 1999). Antibodies of the present invention are useful, for instance, in a variety of diagnostic and therapeutic methods, based on state-in-the-art techniques (see Harlow and Lane, Using Antibodies: A Laboratory Manual, Cold Spring Harbor Laboratory Press, Cold Spring Harbor, N.Y., 1999 and Edwards R., Immunodiagnostics: A Practical Approach, Oxford University Press, Oxford, England, 1999) such as enzyme-immuno assays (e.g. enzyme-linked immunosorbent assay, ELISA), radioimmuno assays, chemoluminescence-immuno assays, Western-blot, immunoprecipitation and antibody microarrays. These methods involve the detection of translation products of the HIF3a gene, or fragments, or derivatives, or variants thereof.

In a preferred embodiment of the present invention, said antibodies can be used for detecting the pathological state of a cell in a sample obtained from a subject, comprising immunocytochemical staining of said cell with said antibody, wherein an altered degree of staining, or an altered staining pattern in said cell compared to a cell representing a known health status indicates a pathological state of said cell. Preferably, the pathological state relates to a neurodegenerative disease, in particular to AD. Immunocytochemical staining of a cell can be carried out by a number of different experimental methods well known in the art. It might be preferred, however, to apply an automated method for the detection of antibody binding, wherein the determination of the degree of staining of a cell, or the determination of the cellular or subcellular staining pattern of a cell, or the topological distribution of an antigen on the cell surface or among organelles and other subcellular structures within the cell, are carried out according to the method described in U.S. Pat. No. 6,150,173.

Other features and advantages of the invention will be apparent from the following description of figures and examples which are illustrative only and not intended to limit the remainder of the disclosure in any way.

### FIGURES

FIGS. 1 and 2 illustrate the verification of the differential expression of the human HIF3a gene, in particular of the HIF3a splice variant 1, in AD brain tissues by quantitative RT-PCR analysis. Quantification of RT-PCR products from RNA samples collected from the frontal cortex (F) and the temporal cortex (T) of AD patients (FIG. 1a) and samples from the frontal cortex (F) and the hippocampus (H) of AD patients (FIG. 2a) was performed by the LightCycler rapid thermal cycling technique. Likewise, samples of healthy, age-matched control individuals were compared (FIG. 1b for frontal cortex and temporal cortex, FIG. 2b for frontal cortex and hippocampus). The data were normalized to the combined average values of a set of standard genes which showed no significant differences in their gene expression levels. Said set of standard genes consisted of genes for cyclophilin B, the ribosomal protein S9, the transferrin receptor, GAPDH, and beta-actin. The figures depict the kinetics of amplification by plotting the cycle number against the amount of amplified material as measured by its fluorescence. Note that the amplification kinetics of HIF3a splice variant 1 cDNAs from both, the frontal and temporal cortices of a normal control individual, and from the frontal cortex and hippocampus of a normal control individual, respectively, during the exponential phase of the reaction are juxtaposed (FIGS. 1b and 2b, arrowheads), whereas in Alzheimer's disease (FIGS. 1a and 2a, arrowheads) there is a significant separation of the corresponding curves, indicating a differential expression of the gene coding for HIF3a, in particular of the HIF3a splice variant 1, in the respective analyzed brain regions, indicating a dysregulation, preferably an upregulation of a transcription product of the human HIF3a gene, in particular of the HIF3a splice variant 1, or a fragment, or derivative, or variant thereof, in the temporal cortex relative to the frontal cortex, and in the hippocampus relative to the frontal cortex.

FIGS. 3, 4 and 5 illustrate the verification of the differential expression of the human HIF3a gene, in particular of the HIF3a splice variant 2 (FIG. 3), of the HIF3a splice variant 3 (FIG. 4) and of the HIF3a splice variant 5 (FIG. 5), respectively, in AD brain tissues by quantitative RT-PCR analysis. Quantification of RT-PCR products from RNA samples collected from the frontal cortex (F) and the temporal cortex (T) of AD patients (FIGS. 3a, 4a, 5a) and likewise, samples collected from healthy, age-matched control individuals (FIGS. 3b, 4b, 5b) was performed by the LightCycler rapid thermal cycling technique. The data were normalized to the combined average values of a set of standard genes which showed no significant differences in their gene expression levels. Said set of standard genes consisted of genes for cyclophilin B, the ribosomal protein S9, the transferrin receptor, GAPDH, and beta-actin. The figures depict the kinetics of amplification by plotting the cycle number against the amount of amplified material as measured by its fluorescence. Note that the amplification kinetics of HIF3a splice variant 2 cDNAs, of HIF3a splice variant 3 cDNAs, and of HIF3a splice variant 5 cDNAs from the frontal and temporal cortices of a normal control individual during the exponential phase of the reaction are juxtaposed (FIGS. 3b, 4b, 5b, arrowheads), whereas in Alzheimer's disease (FIGS. 3a, 4a, 5a, arrowheads) there is a significant separation of the corresponding curves, indicating a differential expression of the gene coding for HIF3a, in particular of the HIF3a splice variant 2, of the HIF3a splice variant 3, and of the HIF3a splice variant 5, in the respective analyzed brain regions, indicating a dysregulation, preferably an upregulation of a transcription product of the human HIF3a gene, in particular of the HIF3a splice variant 2, of the HIF3a splice variant 3, and of the HIF3a splice variant 5, or a fragment, or derivative, or variant thereof, in the temporal cortex relative to the frontal cortex.

FIG. 6 depicts SEQ ID NO. 1, the nucleotide sequence of the 289 bp HIF3a cDNA fragment, identified and obtained by suppressive subtractive hybridization on biochips and by subsequent cloning (sequence in 5′ to 3′ direction).

FIG. 7 outlines the sequence alignment of SEQ ID NO. 1, the 289 bp human HIF3a cDNA fragment, with the nucleotide sequence of the human HIF3a splice variant 1 cDNA, SEQ ID NO. 6 (nucleotides 1421 to 1709).

FIG. 8 discloses SEQ ID NO. 2, the polypeptide sequence of human HIF3a splice variant 1 comprising 450 amino acids. The protein is deduced from a consensus cDNA sequence, SEQ ID NO. 6, constructed on the basis of mRNA and EST sequence information from the Genbank data base as depicted in FIG. 20.

FIG. 9 discloses SEQ ID NO. 3, the polypeptide sequence of human HIF3a splice variant 2 comprising 342 amino acids. The protein is deduced from a consensus cDNA sequence, SEQ ID NO. 7, constructed on the basis of mRNA and EST sequence information from the Genbank data base as depicted in FIG. 21.

FIG. 10 discloses SEQ ID NO. 4, the polypeptide sequence of human HIF3a splice variant 3 comprising 632 amino acids. The protein is deduced from a consensus cDNA sequence, SEQ ID NO. 8, constructed on the basis of mRNA and EST sequence information from the Genbank data base as depicted in FIG. 22.

FIG. 11 discloses SEQ ID NO. 5, the amino acid sequence of human HIF3a splice variant 5 comprising 648 amino acids. The protein is deduced from a consensus cDNA sequence, SEQ ID NO. 9, constructed on the basis of mRNA and EST sequence information from the Genbank data base as depicted in FIG. 23.

FIG. 12 represents SEQ ID NO. 6, the nucleotide sequence of human HIF3a splice variant 1 cDNA, comprising 1709 nucleotides, constructed on the basis of mRNA and EST sequence information from the Genbank data base as depicted in FIG. 20.

FIG. 13 shows SEQ ID NO. 7, the nucleotide sequence of the human HIF3a splice variant 2 cDNA, comprising 2239 nucleotides, as defined by the sequences of mRNAs and ESTs of the Genbank database as depicted in FIG. 21.

FIG. 14 represents SEQ ID NO. 8, the nucleotide sequence of human HIF3a splice variant 3 cDNA, comprising 2082 nucleotides, constructed from the nucleotides of mRNAs and ESTs of the Genbank data base as depicted in FIG. 22.

FIG. 15 represents SEQ ID NO. 9, the nucleotide sequence of human HIF3a splice variant 5 cDNA, comprising 2595 nucleotides, constructed from the nucleotides of mRNAs and ESTs of the Genbank data base as depicted in FIG. 23.

FIG. 16 shows the nucleotide sequence of SEQ ID NO. 10, the coding sequence (cds) of the human HIF3a splive variant 1, comprising 1353 nucleotides (nucleotides 125-1477 of SEQ ID NO. 6).

FIG. 17 shows the nucleotide sequence of SEQ ID NO.11, the coding sequence (cds) of the human HIF3a splive variant 2, comprising 1029 nucleotides (nucleotides 23-1051 of SEQ ID NO. 7).

FIG. 18 shows the nucleotide sequence of SEQ ID NO.12, the coding sequence (cds) of the human HIF3a splive variant 3, comprising 1899 nucleotides (nucleotides 13-1911 of SEQ ID NO. 8).

FIG. 19 shows the nucleotide sequence of SEQ ID NO.13, the coding sequence (cds) of the human HIF3a splive variant 5, comprising 1947 nucleotides (nucleotides 226-2172 of SEQ ID NO. 9).

FIG. 20 schematically charts the assembly of the nucleotide sequence of human HIF3a splice variant 1 cDNA, SEQ ID NO. 6, derived from the alignment of human mRNA sequence with the accession number AK021421 and of human “expressed sequence tags” (ESTs) as found in the Genbank genetic data base. EST and mRNA numbers are written on the left side, all sequences are 5′ to 3′ directed.

FIG. 21 schematically charts the assembly of the nucleotide sequence of human HIF3a splice variant 2 cDNA, SEQ ID NO. 7, derived from the alignment of human mRNA sequence with the accession number BC026308 and of human “expressed sequence tags” (ESTs) as found in the Genbank genetic data base. EST and mRNA numbers are written on the left side, all sequences are 5′0 to 3′ directed.

FIG. 22 schematically charts the assembly of the nucleotide sequence of human HIF3a splice variant 3 cDNA, SEQ ID NO. 8, derived from the alignment of human mRNA sequences with the accession numbers AK021421, AK021653, AK027725, AB054067 and AF463492, and of human “expressed sequence tags” (ESTs) as found in the Genbank genetic data base. EST and mRNA numbers are written on the left side, all sequences are 5′ to 3′ directed.

FIG. 23 schematically charts the assembly of the nucleotide sequence of human HIF3a splice variant 5 cDNA, SEQ ID NO. 9, derived from the alignment of human mRNA sequence with the accession number AK021653 and of human “expressed sequence tags” (ESTs) as found in the Genbank genetic data base. EST and mRNA numbers are written on the left side, all sequences are 5′ to 3′ directed.

FIG. 24 discloses the initial identification of differential expression of the human HIF3a gene by subtractive suppressive microarray hybridization experiments. Identical biochips containing cDNA clones of subtracted AD and control brain cDNA libraries were co-hybridized with different Cyanine3 (Cy3) and Cyanine5 (Cy5) labeled cDNA probes, designated as probes A, B, or C, respectively. Cy3 and Cy5 labeled cDNA probes (A) were generated by labeling cDNAs from frontal or temporal cortex of AD patients and control persons, respectively, refer to section (vi-a) of the example description. Cy3 and Cy5 labeled SMART probes (B) were generated from cDNAs, derived from frontal or temporal cortex of AD patients and control persons, respectively, refer to section (vi-b). Cy3 and Cy5 labeled SSH probes (C) were derived from cDNA populations after suppressive subtractive hybridization of brain cDNAs from frontal and temporal cortex of AD patients and of control individuals, respectively, refer to section (vi) (PF_{SSH(1)}= AD patients frontal cortex cDNA after subtraction of AD patients temporal cortex cDNA; PT_{SSH(2)}= AD patients temporal cortex cDNA after subtraction of AD patients frontal cortex cDNA; CT_{SSH(3)}=control individuals temporal cortex cDNA after subtraction of AD patients temporal cortex cDNA; PT_{SSH(4)}=AD patient temporal cortex cDNA after subtraction of control individual temporal cortex cDNA). The table lists the gene expression level of HIF3a indicated as the ratio of fluorescence intensity measured for the temporal cortex relative to the frontal cortex of AD patients. The ratios of fluorescence intensity reflect a differential regulation of human HIF3a RNA expression in temporal and frontal cortex of AD patients and a relative downregulation of HIF3a transcripts in temporal cortex and upregulation in frontal cortex of AD patients compared to controls.

FIG. 25 lists HIF3a splice variant 1 expression levels in the temporal cortex relative to the frontal cortex in fifteen AD patients, herein identified by internal reference numbers P010, P011, P012, P014, P016, P017, P019, P038, P040, P041, P042, P046, P047, P048, P049 (0.78 to 2.30 fold, values according to the formula described below) and twentyfive healthy, age-matched control individuals, herein identified by internal reference numbers C005, C008, C011, C012, C014, C025, C026, C027, C028, C029, C030, C031, C032, C033, C034, C035, C036, C038, C039, C041, C042, DE02, DE03, DE05, DE07 (0.67 to 1.79 fold, values according to the formula described below). For an up-regulation in the temporal cortex, the values shown are calculated according to the formula described herein (see below) and in case of an up-regulation in the frontal cortex the reciprocal values are calculated, respectively. An obvious difference reflecting an up-regulation in the temporal cortex is shown.The bar diagram visualizes individual natural logarithmic values of the temporal to frontal cortex, In(IT/IF), and of the frontal to temporal cortex regulation factors, In(IF/IT), in different Braak stages (0 to 6).

FIG. 26 lists the gene expression levels in the hippocampus relative to the frontal cortex for the HIF3a splice variant 1 in six Alzheimer's disease patients, herein identified by internal reference numbers P010, P011, P012, P014, P016, P019 (0.70 to 2.46 fold) and three healthy, age-matched control individuals, herein identified by internal reference numbers C004, C005, C008 (0.65 to 1.55 fold). The values shown are calculated according to the formula described herein (see below). The scatter diagram visualizes individual logarithmic values of the hippocampus to frontal cortex regulation ratios, log(ratio HC/IF), in control samples (dots) and in AD patient samples (triangles).

FIG. 27 lists HIF3a splice variant 2 expression levels in the temporal cortex relative to the frontal cortex in fifteen AD patients, herein identified by internal reference numbers P010, P011, P012, P014, P016, P017, P019, P038, P040, P041, P042, P046, P047, P048, P049 (0.55 to 2.38 fold, values according to the formula described below) and twentyfive healthy, age-matched control individuals, herein identified by internal reference numbers C005, C008, C011, C012, C014, C025, C026, C027, C028, C029, C030, C031, C032, C033, C034, C035, C036, C038, C039, C041, C042, DE02, DE03, DE05, DE07 (0.33 to 2.32 fold, values according to the formula described below). For an up-regulation in the temporal cortex, the values shown are calculated according to the formula described herein (see below) and in case of an up-regulation in the frontal cortex the reciprocal values are calculated, respectively. An obvious difference reflecting an up-regulation in the temporal cortex is shown. The bar diagram visualizes individual natural logarithmic values of the temporal to frontal cortex, In(IT/IF), and of he frontal to temporal cortex regulation factors, In(IF/IT), in different Braak stages (0 to 6).

FIG. 28 lists HIF3a splice variant 3 expression levels in the temporal cortex relative to the frontal cortex in fifteen AD patients, herein identified by internal reference numbers P010, P011, P012, P014, P016, P017, P019, P038, P040, P041, P042, P046, P047, P048, P049 (0.77 to 2.56 fold, values according to the formula described below) and twentyfour healthy, age-matched control individuals, herein identified by internal reference numbers C005, C008, C011, C012, C014, C025, C026, C027, C028, C029, C030, C031, C033, C034, C035, C036, C038, C039, C041, C042, DE02, DE03, DE05, DE07 (0.47 to 2.25 told, values according to the formula described below). For an up-regulation in the temporal cortex, the values shown are calculated according to the formula described herein (see below) and in case of an up-regulation in the frontal cortex the reciprocal values are calculated, respectively. A prominent difference reflecting a strong up-regulation in the temporal cortex is shown. The bar diagram visualizes individual natural logarithmic values of the temporal to frontal cortex, In(IT/IF), and of the frontal to temporal cortex regulation factors, In(IF/IT), in different Braak stages (0 to 6).

FIG. 29 lists HIF3a splice variant 5 expression levels in the temporal cortex relative to the frontal cortex in fifteen AD patients, herein identified by internal reference numbers P010, P011, P012, P014, P016, P017, P019, P038, P040, P041, P042, P046, P047, P048, P049 (0.77 to 2.33 fold, values according to the formula described below) and twentyfour healthy, age-matched control individuals, herein identified by internal reference numbers C005, C008, C011, C012, C014, C025, C026, C027, C028, C029, C030, C031, C033, C034, C035, C036, C038, C039, C041, C042, DE02, DE03, DE05, DE07 (0.63 to 1.92 fold, values according to the formula described below). For an up-regulation in the temporal cortex, the values shown are calculated according to the formula described herein (see below) and in case of an up-regulation in the frontal cortex the reciprocal values are calculated, respectively. An obvious difference reflecting an up-regulation in the temporal cortex is shown. The bar diagram visualizes individual natural logarithmic values of the temporal to frontal cortex, In(IT/IF), and of the frontal to temporal cortex regulation factors, In(IF/IT), in different Braak stages (0 to 6).

FIG. 30 shows the analysis of absolute mRNA expression of HIF3a splice variant 1 by comparison of control and AD stages using statistical method of the median at 98%-confidence level. The data were calculated by defining control groups including subjects with either Braak stages 0 to 1, Braak stages 0 to 2, or Braak stages 0 to 3 which are compared with the data calculated for the defined AD patient groups including Braak stages 2 to 6, Braak stages 3 to 6 and Braak stages 4 to 6, respectively. Additionally, three groups including subjects with either Braak stages 0 to 1, Braak stages 2 to 3 and Braak stages 4 to 6, respectively, were compared with each other. A difference was detected comparing frontal cortex (F) and inferior temporal cortex (T) of AD patients and of control persons with each other. Said difference reflects an upregulation of HIF3a sv1 in the temporal cortex and in the frontal cortex of AD patients relative to the temporal cortex and frontal cortex of of control persons which is prominent comparing the Braak stages 0-3 with Braak stages 4-6 with each other. The Braak stages correlate with the progressive course of AD disease which, as shown in the instant invention, is associated with an increasing difference in the regulation, the level and the activity of HIF3a sv1 as described above.

FIG. 31 shows the analysis of absolute mRNA expression of HIF3a splice variant 2 by comparison of control and AD stages using statistical method of the median at 98%-confidence level. The data were calculated by defining control groups including subjects with either Braak stages 0 to 1, Braak stages 0 to 2, or Braak stages 0 to 3 which are compared with the data calculated for the defined AD patient groups including Braak stages 2 to 6, Braak stages 3 to 6 and Braak stages 4 to 6, respectively. Additionally, three groups including subjects with either Braak stages 0 to 1, Braak stages 2 to 3 and Braak stages 4 to 6, respectively, were compared with each other. A difference was detected comparing frontal cortex (F) and inferior temporal cortex (T) of AD patients and of control persons with each other. Said difference reflects a strong upregulation of HIF3a sv2 in the temporal cortex and in the frontal cortex of AD patients relative to the temporal cortex and frontal cortex of of control persons which is prominent comparing the Braak stages 0-3 with Braak stages 4-6 with each other. The Braak stages correlate with the progressive course of AD disease which, as shown in the instant invention, is associated with an increasing difference in the regulation, the level and the activity of HIF3a sv2 as described above.

FIG. 32 shows the analysis of absolute mRNA expression of HIF3a splice variant 3 by comparison of control and AD stages using statistical method of the median at 98%-confidence level. The data were calculated by defining control groups including subjects with either Braak stages 0 to 1, Braak stages 0 to 2, or Braak stages 0 to 3 which are compared with the data calculated for the defined AD patient groups including Braak stages 2 to 6, Braak stages 3 to 6 and Braak stages 4 to 6, respectively. Additionally, three groups including subjects with either Braak stages 0 to 1, Braak stages 2 to 3 and Braak stages 4 to 6, respectively, were compared with each other. A difference was detected comparing frontal cortex (F) and inferior temporal cortex (T) of AD patients and of control persons with each other. Said difference reflects a strong upregulation of HIF3a sv3 in the temporal cortex and in the frontal cortex of AD patients relative to the temporal cortex and frontal cortex of of control persons which is prominent comparing the Braak stages 0-3 with Braak stages 46 with each other. The Braak stages correlate with the progressive course of AD disease which, as shown in the instant invention, is associated with an increasing difference in the regulation, the level and the activity of HIF3a sv3 as described above.

FIG. 33 shows the analysis of absolute mRNA expression of HIF3a splice variant 5 by comparison of control and AD stages using statistical method of the median at 98%-confidence level. The data were calculated by defining control groups including subjects with either Braak stages 0 to 1, Braak stages 0 to 2, or Braak stages 0 to 3 which are compared with the data calculated for the defined AD patient groups including Braak stages 2 to 6, Braak stages 3 to 6 and Braak stages 4 to 6, respectively. Additionally, three groups including subjects with either Braak stages 0 to 1, Braak stages 2 to 3 and Braak stages 4 to 6, respectively, were compared with each other. A difference was detected comparing frontal cortex (F) and inferior temporal cortex (T) of AD patients and of control persons with each other. Said difference reflects a strong upregulation of HIF3a sv5 in the temporal cortex and in the frontal cortex of AD patients relative to the temporal cortex and frontal cortex of of control persons which is prominent comparing the Braak stages 0-3 with Braak stages 46 with each other. The Braak stages correlate with the progressive course of AD disease which, as shown in the instant invention, is associated with an increasing difference in the regulation, the level and the activity of HIF3a sv5 as described above.

FIG. 34 depicts a Western blot image of total cell protein extracts labeled with polyclonal anti-myc antibody (MBL, 1:5000).

Lanes A and B: total protein extract of H4APPsw cells stably expressing HIF3a sv3 tagged with a myc-tag (HIF3a sv3-myc, A) and control H4APPsw cells (B). The arrow indicates a major band at about 70 kDa (lane A), which, corresponds to the predicted molecular weight of the HIF3a sv3 protein.

FIG. 35 shows the immunofluorescence analysis of H4APPsw control cells and H4APPsw cells stably over-expressing the myc-tagged HIF3a sv3 protein (H4APPsw-HIF3a sv3 cds-myc). The HIF3a sv3-myc protein was detected with rabbit polyclonal anti-myc antibodies (MBL) and a Cy3-conjugated anti-rabbit antibody (Amersham) (FIGS. 35A and B). The cellular nucleus was stained with DAPI (FIGS. 35C and D). The overlay analysis indicate that the HIF3a sv3 cds-myc protein is mainly localized to the nucleus (FIG. 35E) and is over-expressed in more than 70% of the H4APPsw-HIF3a sv3 cds-myc transduced cells as compared to the H4APPsw control cells (FIG. 35F).

FIG. 36 depicts sections from human temporal cortex (cortex, CT) labeled with an affinity-purified rabbit polyclonal anti-HIF3a antiserum (HSR1, 1:80) raised against a peptide corresponding to amino acids 290 to 304 present in HIF3a sv1, HIF3a sv3, HIF3a sv5 and a FITC-conjugated goat anti-rabbit IgG antiserum (green signals, FIG. 36 panel A and B, middle and right pictures). Neuronal cells are labeld with the neuron specific marker NeuN (red signals, FIG. 36 panel A) and astrocytes are labeled with the astrocytic marker GFAP (red signals, FIG. 36 panel B). Blue signals indicate nuclei stained with DAPI. The upper panel A shows that the neurons (marker NeuN) exhibit strong nuclear HIF3a immunoreactivity, the yellow arrows examplarily indicate neuronal cells expressing HIF3a (right and middle pictures). The lower panel B shows the staining of astrocytes (marker GFAP), which present only weak nuclear HIF3a immunosignals (yellow arrows, middle and right pictures).

FIG. 37 depicts sections from the white matter (WM) of human temporal cortex labeled with an affinity-purified rabbit polyclonal anti-HIF3a antiserum (HSR1, 1:80) raised against a peptide corresponding to amino acids 290 to 304 present in HIF3a sv1, HIF3a sv3, HIF3a sv5 and a FITC-conjugated goat anti-rabbit IgG antiserum (green signals, FIG. 37 panel A and B, middle and right pictures). Microglial cells are labeld with the microglia specific marker CD68 (red signals,

FIG. 37 panel A) and oligodendrocytes are labeled with the oligodendrocytic marker CNPase (red signals, FIG. 37 panel B). Blue signals indicate nuclei stained with DAPI. The upper panel A shows that the microglia (marker CD68) exhibit nuclear HIF3a immunoreactivity, the yellow arrows examplarily indicate micoglial cells expressing HIF3a (right and middle pictures). The lower panel B shows the staining of oligodendrocytes (marker CNPase), which show moderate nuclear HIF3a immunosignals (yellow arrows, right and middle pictures), which is not co-localized with myelin.

FIG. 38 exemplarily depicts micrographs digitally taken from sections of the inferior temporal gyrus (CT, lower panel) and of the frontal cortex (CF) from control donors (Control Braak 1 (C029)), from persons with middle Braak stage (Braak 3 (C035)), and from Alzheimer's patients (Patient Braak 4 (P016), Braak 5 (P011), Braak 6 (P017)). The tissue sections are immunolabeled with affinity-purified rabbit polyclonal anti-HIF3a antiserum (HSR1) (green signals) (magnification 40×). Astrocytes are stained with an antibody against the astrocytic-specific marker GFAP (red signal). The nucleus is stained with DAPI (blue signal). As compared to low-Braak controls (Braak 1), nuclear astrocytic HIF3a immunoreactivity is increased, the level of HIF3a translation product, of HIF3a protein is increased in the temporal cortex of persons with middle Braak stages (Braak 3 and 4), as well as in both the frontal and the temporal cortex of persons having high Braak stages (Braak 5 and 6). The finding that HIF3a immunoreactivity, the level of HIF3a translation product, of HIF3a protein is increased in the temporal cortex from middle-Braak-stage persons, and at even higher Braak stages in both the frontal and the temporal cortex, indicates that the course of AD, the progression of neurofibrillary pathology, is reflected by an elevated astrocytic HIF3a expression which may either accompany or followed or even preceded AD neurodegenerative changes. The data exemplarily shown here clearly indicate that the level of intensity and quantity of astrocytic immunoreactivity of the HIF3a protein is increased in the inferior temporal cortex and in the frontal cortex from patients (Braak stages 4 to 6) as compared to the inferior temporal cortex and/or the frontal cortex from control persons (Braak 1) and persons having middle Braak stage (Braak 3), that the level of HIF3a protein is increasing with increasing Braak stages of AD. Temporal cortex (CT); Frontal cortex (CF); Healthy control person (Control); Alzheimer's patient (Patient).

FIG. 39 depicts the comparison of the expression efficiency of three different HIF3a sv3-myc expressing fly lines. The efficiency is calculated according to the cycle number and efficiency of the RT-PCR reaction of the HIF3a sv3-myc specific primer pair. Measurements were done in triplicates for each genotype. Genotypes used: w; UAS-HIF3a-sv3-myc#3/gmr-GAL4; w; UAS-HIF3a-sv3-myc#4/gmr-GAL4; w; UAS-HIF3a-sv3-myc#57/+; gmr-GAL4/+.

FIG. 40 HIF3a sv3 protein localizes to nuclei of photoreceptor cells in the adult retina. Transgenic HIF3a-sv3 fly line #3 was expressed under the control of gmr-GAL4. Cryostat sections through the adult eye were stained with anti-myc (MYC, red signal) and DAPI (DAPI, blue signal) to label nuclei. Wild-type non-transgenic flies were used as negative controls. Arrowheads point to nuclei with overlapping myc- and DAPI-positive signals. Re: retina.

FIG. 41 shows western blots of head homogenates of flies expressing HIF3a-sv3. Three different transgenic fly lines (#3, #4, and #14, lane2, 3 and 4) were used to express HIF3a sv3 under the control of gmr-GAL4 and expression was compared to non-transgenic w1118 flies (lane 1). Equal amounts of protein were loaded in lane 1 to 4. Lane 5: protein extract of H4-APPsw cells stably transfected with HIF3a-sv3-myc construct. Lane 6: protein extract of H4-APPsw cells transiently transfected with HIF3a-sv3-myc construct. Lane 7: H4-APPsw control cells. Blot was probed with a polyclonal anti-myc antibody (MBL). Red asterisks point to low expression of HIF3a sv3 in protein extracts of transgenic flies and H4-neuroglioma cells.

FIG. 42 point to the rescue of photoreceptor cell degeneration in flies expressing hAPP and hBACE under the control of gmr-GAL4. 10 μm cryostat sections of the adult brain and complex eye were stained with the photoreceptor cell specific antibody 24B10 8 days after eclosion. (A) Age matched control flies expressing hAPP and hBACE show age-dependent degeneration of photoreceptor cells in the retina. (B) Co-expression of HIF3a-sv3 (#3, #4, #57) rescues photoreceptor cell degeneration as judged by the integrity of the retina. (C) Strong suppression of photoreceptor cell degeneration in 3 day old flies co-expressing hAPP, hBACE, DPsnL235P and HIF3a-sv3#57. Re: retina; La: lamina; Me: medulla. Scale bar: 50 μm.

FIG. 43 shows a western blot of Abeta immunoprecipitated from homogenates of flies expressing hAPP/hBACE and hAPP/hBACE/HIF3a-sv3. Monomeric synthetic Abeta40 is detected at a molecular weight of 4 kDa whereas Abeta peptides isolated from fly homogenates are detected by antibody 6E10 as a dimer. Homogenates of fifteen flies per lane were immunoprecipitated using antibody 6E10. Equal amounts of fly protein were used for each immunoprecipitation. No difference in Abeta level between control flies and flies co-expressing HIF3a sv3 could be detected.

FIG. 44 pictures Thioflavin S positive plaques (indicated by arrows) on paraffin sections through the retina of 42 day old male flies expressing hAPP/hBACE/ DPsnL235P (panel A) or hAPP/hBACE/DPsnL235P/HIF3a-sv3#4 (panel B). No difference in the onset of plaque formation was detected between control flies and hAPP/hBACE/DPsnL235P/HIF3a-sv3#4 expressing flies. Ten heads per genotype were sectioned and stained in parallel. Magnification: 20×; Insets: 100×; Scale bar: 10 μm.

### EXAMPLE

(i) Brain Tissue Dissection from Patients with AD:

Brain tissues from AD patients and age-matched control subjects (temporal cortex, T, frontal cortex, F, hippocampus, H) were collected on average within 5 hours post-mortem and immediately frozen on dry ice. Sample sections from each tissue were fixed in paraformaldehyde for histopathological confirmation of the diagnosis. Brain areas for differential expression analysis were identified and stored at −80° C. until RNA extractions were performed.

(ii) Isolation of Total mRNA:

Total RNA was extracted from post-mortem brain tissue by using the RNeasy kit (Qiagen) according to the manufacturer's protocol. The accurate RNA concentration and the RNA quality were determined with the DNA LabChip system using the Agilent 2100 Bioanalyzer (Agilent Technologies). For additional quality testing of the prepared RNA, i.e. exclusion of partial degradation and testing for DNA contamination, specifically designed intronic GAPDH oligonucleotides and genomic DNA as reference control were used to generate a melting curve with the LightCycler technology as described in the manufacturer's protocol (Roche).

(iii) cDNA Synthesis and Rsa I Digestion

In order to identify changes in gene expression in different tissues, a screening method combining cDNA synthesis, suppressive subtractive hybridization (SSH) and screening of microarray chips with a diversity of cDNA probes from SSH was employed. This technique compares different populations of mRNA and provides clones of genes that are expressed in one population of cells but not, or at lower level, in the other population of cells. In the present invention, RNA populations from selected post-mortem brain tissues (frontal and temporal cortex) of AD patients and age-matched control subjects were corn pared.

As starting material for the suppressive subtractive microarray analysis total RNA was extracted as described above (ii). For production of preferably full-length cDNAs, the polymerase chain reaction (PCR)-based method ‘SMART cDNA Synthesis’ was performed according to the manufacturer's protocol (Clontech).

The principle of ‘SMART cDNA synthesis’ has been described in detail (Chenchik et al., in Gene Cloning and Analysis by RT-PCR, eds. Siebert and Larrick, Biotechniques Books, Natick 1998:305-320). For SMART cDNA synthesis, four RNA pools, each consisting of 8 μg total RNA, were prepared. Each pool contained 2 μg of each of four different samples, i.e. from inferior frontal cortex (CF) and from inferior temporal cortex (CT) of control brains, from inferior frontal cortex (PF) and from inferior temporal cortex (PT) of patient brains, respectively. An amount of 1 μg of total RNA mix was utilized in a reaction volume of 50 μl (PCR cycler: Multi Cycler PTC 200, MJ Research). The second SMART PCR step was performed using 19 cycles. SuperScript II RNaseH Reverse Transcriptase and 5× first-strand buffer (Invitrogen) were used.

After extraction and purification of the PCR products, restriction digestions were carried out with 30 U Rsa I (MBI Fermentas) for 2.5 hours at 37° C. Rsa I restriction sites are located within the universal priming sites of the double stranded (ds) cDNA. The quality of the digestions was analyzed by agarose gel electrophoresis, the digested samples were purified (QlAquick PCR Purification Kit, Qiagen), and the cDNA concentrations were determined by UV spectrophotornetry (Biorad).

(iv) Suppressive Subtractive Hybridization (SSH)

Four SMART cDNA pools (iii) were compared using suppressive subtractive hybridization. A pool of cDNA containing differentially expressed genes is thereby designated as “Tester”, the reference cDNA pool as “Driver”. The two pools are hybridized, and all cDNAs, present in both pools, will be eliminated, i.e. the Driver-pool will be subtracted from the Tester-pool. Thus, clones of genes that are predominantly expressed in the Tester population are obtained.

The ‘PCR-Select cDNA Subtraction Kit’ was used to perform the subtractive hybridization (Clontech). The ‘Tester’ SMART cDNA pools, derived from frontal and temporal cortex (CF and CT) of control brains, and from frontal and temporal cortex (PF and PT) of patient brains (iii), were subdivided into two pools each. Each pool was ligated with Adaptor 1 or Adaptor 2, respectively, thus obtaining 6 different ‘Tester’ cDNA pools. The three ‘Driver’ SMART cDNA pools, CT, PF and PT, remained unligated. In a first hybridization step, used to enrich for differentially expressed sequences, the following three different ‘Tester’ SMART cDNA pools were combined with an excess of the following ‘Driver’ SMART cDNAs: SSH(1): PF-‘Tester’ and PT-‘Driver’; SSH(2): PT-‘Tester’ and PF-‘Driver’; SSH(3): CT-‘Tester’ and PT-‘Driver’; SSH(4): PT-‘Tester’ and CT-‘Driver’. Following a denaturation step for 1.5 min at 98° C., the hybridization was carried out for 8 hours at 68° C. In a second step, the two corresponding primary hybridization samples of ‘Tester’ SMART CDNA pools ligated to Adaptor 1 or 2, respectively, were mixed and re-hybridized at 68° C. for 15 hours, with an excess of the ‘Driver’ SMART CDNA pool, as used before. Thus, suitable double stranded cDNAs for subsequent amplification, i.e. with both Adaptor sequences at their 5′ and 3′ ends and therefore with different annealing sites, were generated. The following PCR steps were applied to obtain efficiently amplified specific products and to suppress nonspecific amplification. In the first PCR, missing strands of the adaptors were filled in by DNA-polymerase activity. 1 μl of the obtained hybridization products each were subjected to PCR using the corresponding ‘Primer 1’ (10 μM) (Clontech) along with 1×PCR reaction buffer (Clontech), 10 mM dNTP-Mix (dATP, dGTP, dCTP, dTTP, Amersham Pharmacia Biotech), and 0.5 μl 50× Advantage CDNA Polymerase Mix (Clontech) in a 25 μl final volume. PCR conditions were set as follows: one round at 75° C. for 5 min, which was followed by 27 or 30 cycles: 94° C. for 30 sec, 64° C. or 66° C. for 30 sec, 72° C. for 1.5 min. One final step at 72° C. for 5 min was added to the last cycle. A second nested PCR was performed as described for the first PCR, except that instead of ‘Primer 1’ the nested primers ‘Nested Primer 1’ and ‘2R’ were used and an annealing temperature of 66° C. or 68° C. and 12 or 15 cycles, were applied. PCR-products obtained by different conditions were pooled for subsequent analysis. For the primer sequences used, refer to appendix B of the supplier's user manual (Clontech).

(v) Cloning of Subtracted PCR Products and Production of DNA-biochips

The SSH SMART double stranded cDNAs of the four different combinations SSH(1)-SSH(4), refer to (iv), were ligated into the pCR2.1-vector and transformed into INValphaF' cells according to the manufacturer's instructions (TA Cloning Kit, Invitrogen). Bacterial colonies were picked and analyzed by colony PCR on MTPs (microtiter plates, 96 well, Abgene), using ‘Nested Primer 1’ and ‘Nested Primer 2’. Those MTPs showing more than 90% positive clones were subjected to a preparative colony PCR approach. Per well, the following PCR mix was generated: the corresponding oligonucleotides ‘Nested Primer 1’ and ‘Nested Primer 2’ (0.5 μM each), 1× Titanium PCR buffer (Clontech), 200 μM dNTP-Mix (Amersham Pharmacia Biotech), 0.2 × TitaniumTaq DNA-Polymerase (Clontech) in a 120 μl final volume. PCR conditions were set as follows: one round at 94° C. for 30 sec for denaturing, the next round was followed by 35 cycles: 94° C. for 30 sec and 68° C. for 3 min. The quality of the amplified products was checked and analyzed (DNA LabChip system, Agilent 2100 Bioanalyzer, Agilent Technologies), followed by purification (Multiscreen-PCR-Purification system, Millipore).

Additionally, the following standard control samples were generated: three different Arabidopsis thaliana genes, polyA-DNA, salmon sperm DNA, human Cot-1 DNA, and 3×SSC-buffer were used as negative controls (Microarray Validation System, Stratagene); beta-Actin and Xenopus cDNA were used as normalizing contro Is. Several MTPs were made of each of the SSH combinations SSH(1)-(4), harboring amplification products of 96 different clones per plate. The amplified products were spotted in triplicates onto GAPS glass-slides (CMT-GAPS, Corning) by GeneScan Europe.

(vi) Probe Synthesis and Identification of Differentially Expressed Genes by Screening of DNA Biochips

A: cDNA Probe Synthesis

As starting material for the generation of Cyanine3 (Cy3) and Cyanine5 (Cy5) labeled cDNA probes total RNA was extracted and used as described above (ii) and (iii). Two samples of a mix of 2 μg of total RNA and additionally 2 ng Xenopus RNA (standard RNA) per labeling reaction were subjected to a specific reverse transcriptase reaction, whereby the polyA-mRNA is converted into fluorescein-12-dCTP (FL) or biotin-11-dCTP (B) labeled cDNA. The RNA samples derived from frontal cortex (CF) of control brains were labeled with fluorescein, the RNA from the temporal cortex (PT) of patient brains with biotin, respectively. The cDNA reactions were performed according to the Micromax TSA Labeling protocol (NEN Life Science). The purified cDNA probes were resuspended in hybridization buffer and denatured for 7 min at 100° C. Subsequently, half the volume of the fluorescein-labeling reaction (i.e. 1 μg RNA) and half the amount of the biotin-probe were mixed together in 5×SSC, 0.1% SDS, 25% formamide buffer, and applied evenly onto one prehybridized (5×SSC, 0.1% SDS, 1% BSA, 45 min at 42° C.) microarray. Array hybridization was performed over night at 42° C. Following a high stringency wash step, the detection of the bound fluorescein- and biotin labeled probes was performed according to the instructions of the TSA Detection Kit protocol (NEN Life Science). Thereby, in a first step, anti-FL-HRP (fluorescein-horseradish peroxidase) binds to the FL-labeled cDNA probe, and HRP catalyzes the deposition of the fluorescent reporter Cy3 tyramide. In a second step, streptavidin-HRP binds to the B-labeled cDNA probe and catalyzes the deposition of the fluorescent reporter molecule Cy5 tyramide. Biochip 3 was hybridized with cDNA mix PF(Cy3) and PT(Cy5). Scanning the microarrays with the appropriate wavelengths (635 nm, 532 nm) allowed detection of both cyanine dyes simultaneously.

B: SMART Probe Synthesis

For the production of Cyanine3 (Cy3) and Cyanine5 (Cy5) labeled SMART cDNA-probes the PCR-based method ‘SMART cDNA Synthesis’ was performed as described in section (iii). Here we used total RNA as starting material which was extracted as described above (ii). Four RNA mixtures were prepared as described in section (iii). 1 μg of each RNA mix and 1 ng Xenopus total RNA were subjected to the SMART cDNA reaction. For PCR amplification, extraction and purification of the cDNAs, restriction digestion with Rsa I and subsequent purification of the digested samples, refer to section (iii).

SMART cDNA samples were labeled with either Cy3 or Cy5 (Atlas Glass Fluorescent Labeling Kit, Clontech). In the first labeling step, aliphatic amino groups, i.e. aminoallyl-dUTP (Clontech), were incorporated into denatured (100° C., 7 min) Rsa I digested PCR products. The reaction was catalyzed by the Klenow Fragment (MBI Fermentas). In a second labeling step, the fluorescent reporter dyes Cy3 or Cy5 were coupled to the incorporated functionalities. The purified Cy3 and Cy5 labeled SMART cDNA probes (Atlas NucleoSpin Extraction Kit, Clontech) were resuspended in hybridization buffer (5×SSC, 0.1% SDS, 25% formamide) after denaturation for 7 min at 100° C. Subsequently, the Cy3 labeled SMART probe was mixed with the Cy5 labeled SMART probe and together applied evenly onto one prehybridized (5×SSC, 0.1% SDS, 1% BSA, 45 min at 42° C.) microarray. Array hybridization was performed over night at 42° C. High stringency washing of the biochips followed according to the instructions of the TSA Detection Kit protocol (NEN Life Science). Biochips 2 and 7 were hybridized with SMART cDNA mix PF(Cy3) and PT(Cy5). Scanning the microarrays with the appropriate wavelengths (635 nm, 532 nm) allowed detection of both cyanine dyes simultaneously.

C: Subtraction Probe Synthesis

For the production of Cyanine3 (Cy3) and Cyanine5 (Cy5) labeled SSH cDNA-probes, the PCR-based method ‘SMART cDNA Synthesis’ was performed as described in section (iii). Here we used total RNA as starting material which was extracted as described above (ii). Four RNA mixtures were prepared as disclosed in section (iii). 1 μg of each RNA mix was subjected to the SMART cDNA reaction. For PCR amplification, extraction and purification of the cDNAs, restriction digestion with Rsa I and subsequent purification of the digested samples, refer to section (iii). For subtractive hybridization, the PCR-Select cDNA Subtraction Kit (Clontech) was utilized as described in detail in section (iv). The subtracted PCR products of the combinations SSH(1) and SSH(2), and of SSH(3) and SSH(4), respectively, were purified (StrataClean Kit, Stratagene), and Adaptor 1 and 2 removed by restriction digest with Rsa I and Sma I (MBI Fermentas). The SSH cDNA pools were labeled with either Cy3 or Cy5 (Atlas Glass Fluorescent Labeling Kit, Clontech). In the first labeling step, aliphatic amino groups, i.e. aminoallyl-dUTP (Clontech), were incorporated into the denatured (100° C., 7 min) Rsa I and Sma I digested SSH cDNA products. The reaction was catalyzed by the Klenow Fragment (MBI Fermentas). In a second labeling step, the fluorescent reporter dyes Cy3 and Cy5 were coupled to the incorporated functionalities.

The purified Cy3 and Cy5 labeled SSH cDNA probes (for purification refer to the Atlas NucleoSpin Extraction Kit, Clontech) were resuspended in hybridization buffer (5×SSC, 0.1% SDS, 25% formamide) after denaturation for 7 min at 100° C. Subsequently, the Cy3 labeled SSH1 probe was mixed with the Cy5 labeled SSH2 probe, and the Cy3 labeled SSH3 probe with the Cy5 labeled SSH4 probe, respectively. Each combination was applied evenly onto one prehybridized (5×SSC, 0.1% SDS, 1% BSA, 45 min at 42° C.) microarray. Array hybridization was performed over night at 42° C. High stringency washing of the biochips followed according to the instructions of the TSA Detection Kit protocol (NEN Life Science). Biochips 1 and 4 were hybridized with the cDNA mix SSH(1)(Cy3) and SSH(2)(Cy5), and with the mix SSH(3)(Cy3) and SSH(4)(Cy5), respectively. Scanning of the microarrays with the appropriate wavelengths (635 nm, 532 nm) allowed detection of both cyanine dyes simultaneously.

(vii) DNA Biochips Data Evaluation

Fluorescence raw data for Cy3 and Cy5, measured at 635 and 532 nm, respectively, were taken severalfold (for each of the three spots per cDNA). One set of measurements was performed within the spot area (signal), and another set of measurements was taken nearby (background). Subsequently the net fluorescence intensity (Fl₆₃₅, Fl₅₃₂) of the spots was calculated as follows:
Fl_{635/532}=(M Flₛₚₒₜ−1SD Flₛₚₒₜ)−(MFl_{background}+1SDFl_{background}).

In this calculation, M defines the median of the replicate measurements per spot, SD the standard deviation of the corresponding mean. Subsequently, only Fl₆₃₅ and Fl₅₃₂ values of >2 were considered for further evaluation plus those Fl₆₃₅ and Fl₅₃₂ values of <2 where the corresponding value for the second wavelength was >3.

In an analogous manner, the corresponding values for the Xenopus CDNA control and the set of standard (housekeeping) genes were evaluated. The Xenopus cDNA was used as an internal calibrator for the efficiency of cDNA synthesis of the disease relevant mRNAs. Then, from the background corrected Fl_{635/532} medians of the three replicate spots, the statistical mean was calculated and the signal ratio R for the cDNA probes was derived using formula:
R_{635/532}=Fl₆₃₅, calibrated/Fl₅₃₂, calibrated.

In a last step of evaluation, the results of the different hybridizations were considered for logical coherence.

(viii) Confirmation of differential by quantitative RT-PCR analysis:

Positive corroboration of the expression levels of the human HIF3a gene in temporal cortex versus frontal cortex and in the hippocampus versus frontal cortex were analyzed using the LightCycler technology (Roche). This technique features rapid thermal cyling for the polymerase chain reaction as well as real-time measurement of fluorescent signals during amplification and therefore allows for highly accurate quantification of RT-PCR products by using a kinetic, rather than endpoint readout. The ratios of HIF3a cDNAs from temporal cortices of AD patients and of healthy age-matched control individuals, from the frontal cortices of AD patients and of healthy age-matched control individuals, from the hippocampi of AD patients and of age-matched control individuals, and the ratios of HIF3a cDNAs from the temporal cortex and frontal cortex of AD patients and of healthy age-matched control individuals, and the ratios of HIF3a cDNAs from the hippocampus and from frontal cortex of AD patients and of healthy age-matched control individuals, respectively, were determined (relative quantification).

The mRNA expression profiling between frontal cortex tissue (F) and inferior temporal cortex tissue (T) of HIF3a has been analyzed in four up to nine tissues per Braak stage. Because of the lack of high quality tissues from one donor with Braak 3 pathology, tissues of one additional donor with Braak 2 pathology were included, and because of the lack of high quality tissues from one donor with Braak 6 pathology, tissue samples of one additional donor with Braak 5 pathology were included.

For the analysis of the profiling, two general approaches have been applied. Both comparative profiling studies, frontal cortex against inferior temporal cortex as well as control against AD patients, which contribute to the complex view of the relevance of HIF3a in AD physiology, are shown in detail below.

1) Relative comparison of the mRNA expression between frontal cortex tissue and inferior temporal cortex tissue of controls and of AD patients.

This approach allowed to verify that HIF3a is either involved in the protection of the less vulnerable tissue (frontal cortex) against degeneration, or is involved in or enhances the process of degeneration in the more vulnerable tissue (inferior temporal cortex).

First, a standard curve was generated to determine the efficiency of the PCR with specific primers for the HIF3a splice variant 1 encoding gene: 5′-GGGCTCAAGTGATCCTCCTACTT-3′ (nucleotides 1466-1488 of SEQ ID NO. 6) and 5′-CATGATGGCACATAGCTGCAGT-3′ (nucleotides 1510-1531 of SEQ ID NO. 6) and with specific primers for the HIF3a splice variant 2 encoding gene: 5′-TTTGCGTGAACCTCTGCTTAAG-3′ (nucleotides 1305-1326 of SEQ ID NO. 7) and 5′-CACCATGCCAGGCCAAAT-3′ (nucleotides 1360-1377 of SEQ ID NO. 7) and with specific primers for the HIF3a splice variant 3 encoding gene: 5′-TCTCTGGCCCTCATTACCTAGCT-3′ (nucleotides 1866-1888 of SEQ ID NO. 8) and 5′-CTGTATGACCCTCAACCAGCC-3′ (nucleotides 1935-1955 of SEQ ID NO. 8) and with specific primers for the HIF3a splice variant 5 encoding gene: 5′-ACTCTTGGTCTCCCACAGGAAA-3′ (nucleotides 2318-2339 of SEQ ID NO. 9) and 5′-AACAGAGCGAGCAGTGCCTT-3′ (nucleotides 2380-2399 of SEQ ID NO. 9). PCR amplification (95° C. and 1 sec, 56° C. and 5 sec, and 72° C. and 5 sec) was performed in a volume of 20 μl containing LightCycler-FastStart DNA Master SYBR Green I mix (contains FastStart Taq DNA polymerase, reaction buffer, dNTP mix with dUTP instead of dTTP, SYBR Green I dye, and 1 mM MgCl₂; Roche), 0.5 μM primers, 2 μl of a cDNA dilution series (final concentration of 40, 20, 10, 5, 1 and 0.5 ng human total brain cDNA; Clontech) and, depending on the primers used, additional 3 mM MgCl₂. Melting curve analysis revealed a single peak at approximately 83.5° C. for the HIF3a splice variant 1 gene specific primers, at 78° C. for the HIF3a splice variant 2 gene specific primers, at 82° C. for the HIF3a splice variant 3 gene specific primers and at about 85° C. for the HIF3a splice variant 5 gene specific primers, with no visible primer dimers. Quality and size of the PCR product were determined with the DNA LabChip system (Agilent 2100 Bioanalyzer, Agilent Technologies). A single peak at the expected size of 66 bp for the HIF3a splice variant 1 gene, at 73 bp for the HIF3a splice variant 2 gene, at 90 bp for the HIF3a splice variant 3 gene and at 82 bp for the HIF3a splice variant 5 gene was observed in the electropherogram of the sample.

In an analogous manner, the PCR protocol was applied to determine the PCR efficiency of a set of reference genes which were selected as a reference standard for quantification. In the present invention, the mean value of five such reference genes was determined: (1) cyclophilin B, using the specific primers 5′-ACTGAAGCACTACGGGCCTG-3′ and 5′-AGCCGTTGGTGTCTTTGCC-3′ except for MgCI₂ (an additional 1 mM was added instead of 3 mM). Melting curve analysis revealed a single peak at approximately 87° C. with no visible primer dimers. Agarose gel analysis of the PCR product showed one single band of the expected size (62 bp). (2) Ribosomal protein S9 (RPS9), using the specific primers 5′-GGTCAAATTTACCCTGGCCA-3′ and 5′-TCTCATCAAGCGTCAGCAGTTC-3′ (exception: additional 1 mM MgCI₂ was added instead of 3 mM). Melting curve analysis revealed a single peak at approximately 85° C. with no visible primer dimers. Agarose gel analysis of the PCR product showed one single band with the expected size (62 bp). (3) beta-actin, using the specific primers 5′-TGGAACGGTGAAGGTGACA-3′ and 5′-GGCAAGGGACTTCCTGTAA-3′. Melting curve analysis revealed a single peak at approximately 87° C. with no visible primer dimers. Agarose gel analysis of the PCR product showed one single band with the expected size (142 bp). (4) GAPDH, using the specific primers 5′-CGTCATGGGTGTGAACCATG-3′ and 5′-GCTAAGCAGTTGGTGGTGCAG-3′. Melting curve analysis revealed a single peak at approximately 83° C. with no visible primer dimers. Agarose gel analysis of the PCR product showed one single band with the expected size (81 bp). (5) Transferrin receptor TRR, using the specific primers 5′-GTCGCTGGTCAGTTCGTGATT-3′ and 5′-AGCAGTTGGCTGTTGTACCTCTC-3′. Melting curve analysis revealed a single peak at approximately 83° C. with no visible primer dimers. Agarose gel analysis of the PCR product showed one single band with the expected size (80 bp).

For calculation of the values, first the logarithm of the cDNA concentration was plotted against the threshold cycle number Cₜ for the gene coding for HIF3a, i.e. for the HIF3a splice variant 1, HIF3a splice variant 2, HIF3a splice variant 3 and for the HIF3a splice variant 5, respectively, and the five reference standard genes. The slopes and the intercepts of the standard curves (i.e. linear regressions) were calculated for all genes. In a second step, cDNAs from frontal cortex, temporal cortex and hippocampus were analyzed in parallel and normalized to cyclophilin B. The Cₜ values were measured and converted to ng total brain cDNA using the corresponding standard curves:
10ˆ((C₁value−intercept)/slope)[ng total brain cDNA]

The values for temporal and frontal cortex and the values for hippocampus and frontal cortex of HIF3a cDNAs (i.e. of the HIF3a splice variant 1, HIF3a splice variant 2, HIF3a splice variant 3 and/or HIF3a splice variant 5, respectively) and the values from the frontal cortex HIF3a cDNAs of AD patients (P) and control individuals (C), and the values for temporal cortex HIF3a cDNAs of AD patients (P) and of control individuals (C) were normalized to cyclophilin B and the ratios were calculated according to formulas:

$$Ratio = \frac{HIF 3 a temporal [ ng ] \text{/} cyclophilin B temporal [ ng ]}{HIF 3 a frontal [ ng ] \text{/} cyclophilin B frontal [ ng ]}$$

$$Ratio = \frac{HIF 3 a hippocampus [ ng ] \text{/} cyclophilin B hippocampus [ ng ]}{HIF 3 a frontal [ ng ] \text{/} cyclophilin B frontal [ ng ]}$$

$$Ratio = \frac{HIF 3 a ( P ) temporal [ ng ] \text{/} cyclophilin B ( P ) temporal [ ng ]}{HIF 3 a ( C ) temporal [ ng ] \text{/} cyclophilin B ( C ) temporal [ ng ]}$$

$$Ratio = \frac{HIF 3 a ( P ) frontal [ ng ] \text{/} cyclophilin B ( P ) frontal [ ng ]}{HIF 3 a ( C ) frontal [ ng ] \text{/} cyclophilin B ( C ) frontal [ ng ]}$$

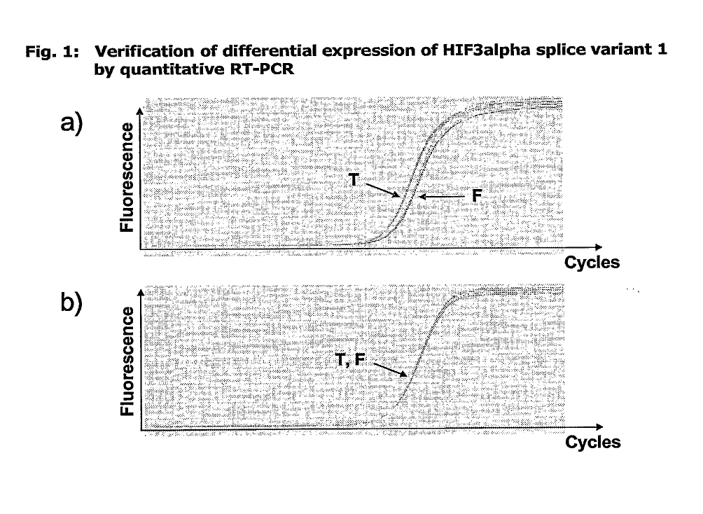

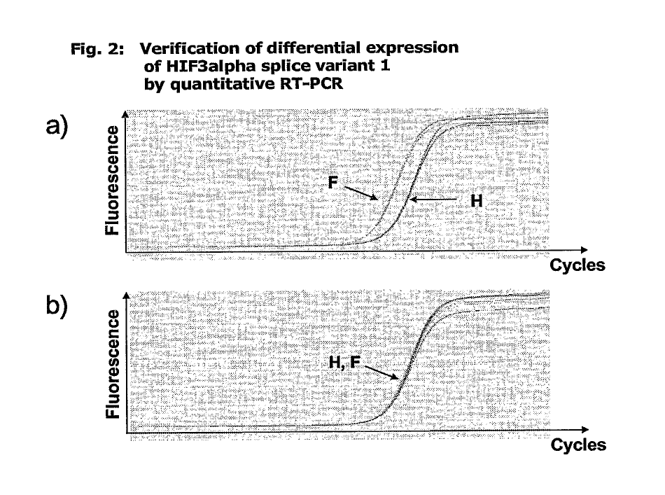

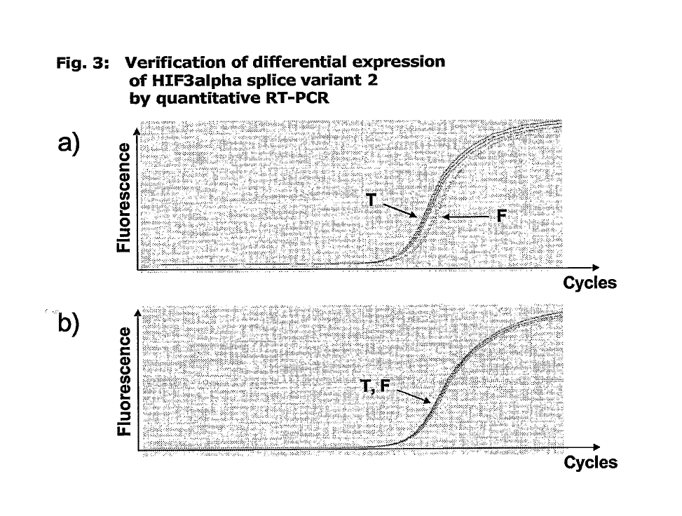

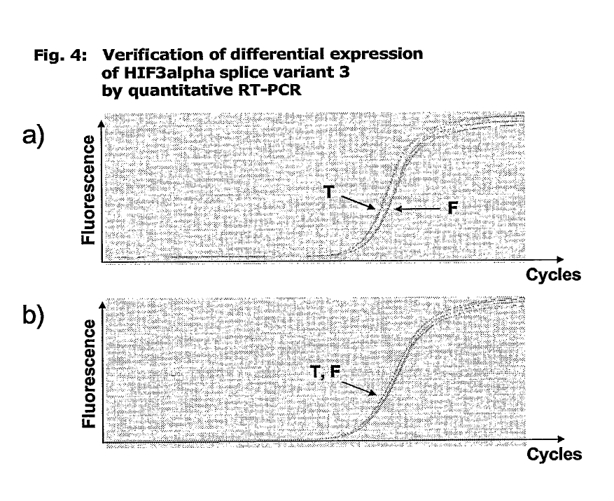

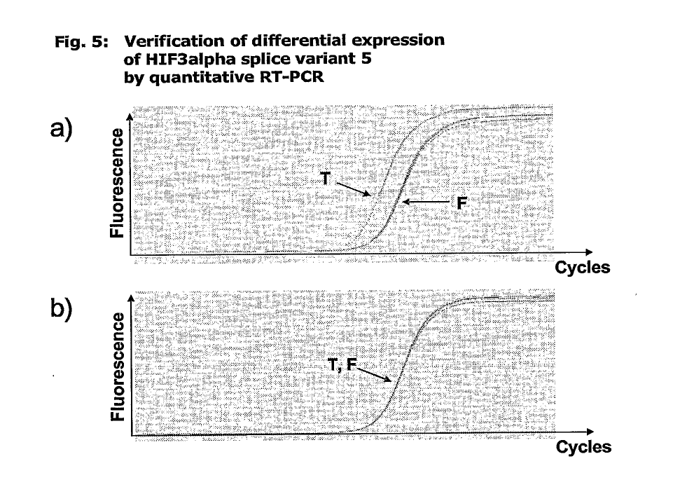

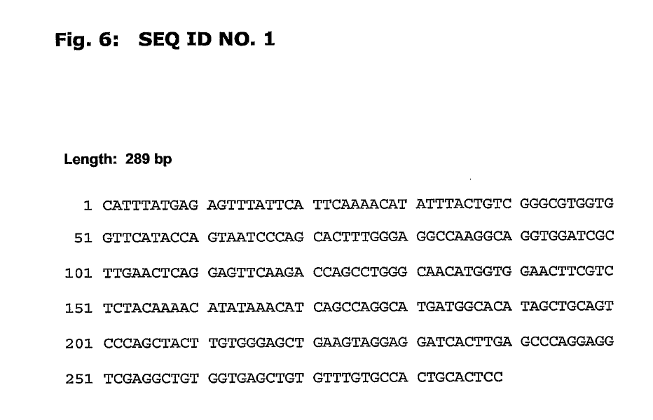

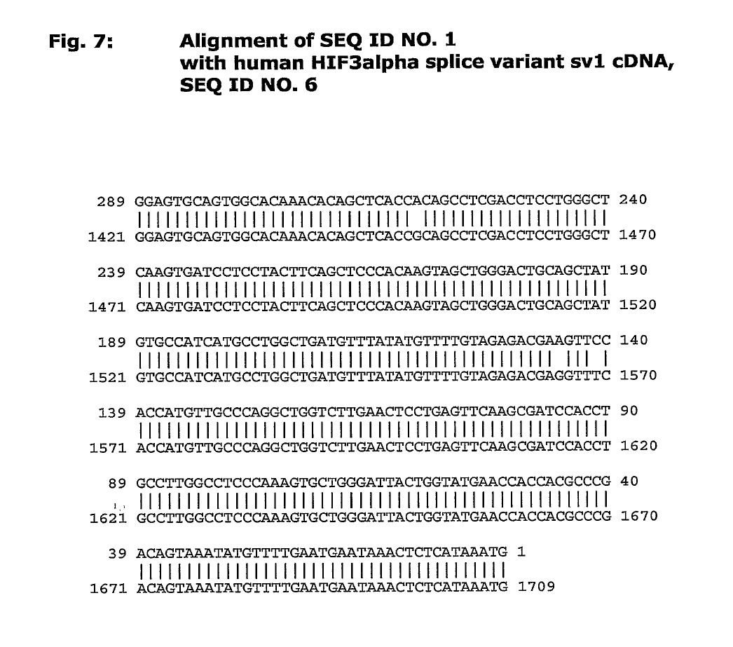

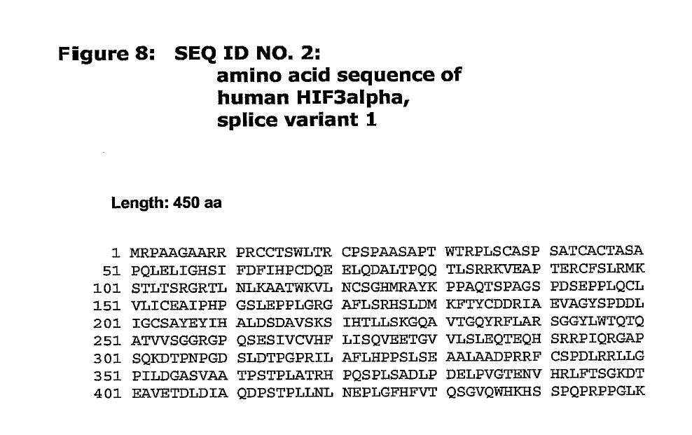

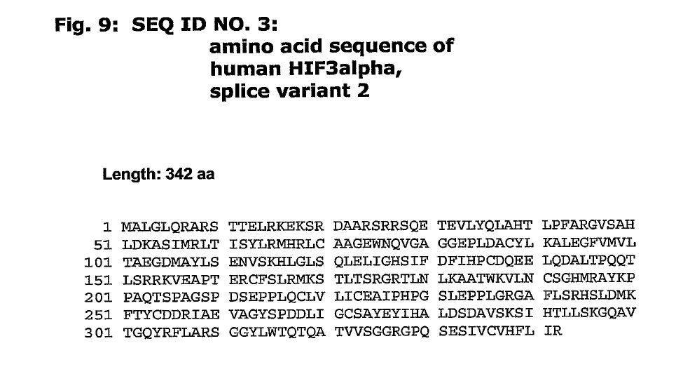

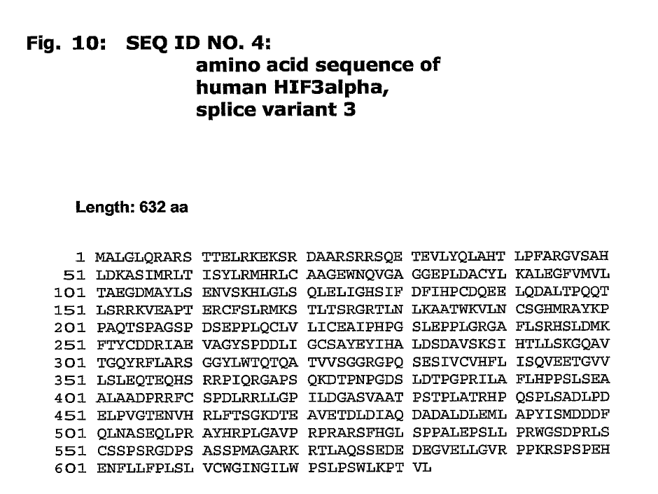

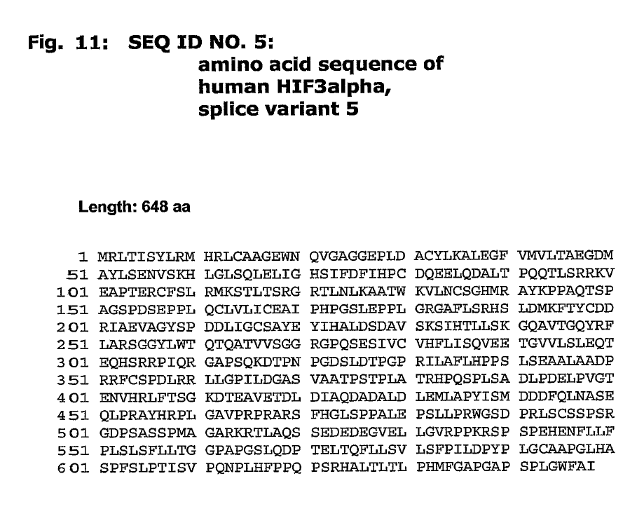

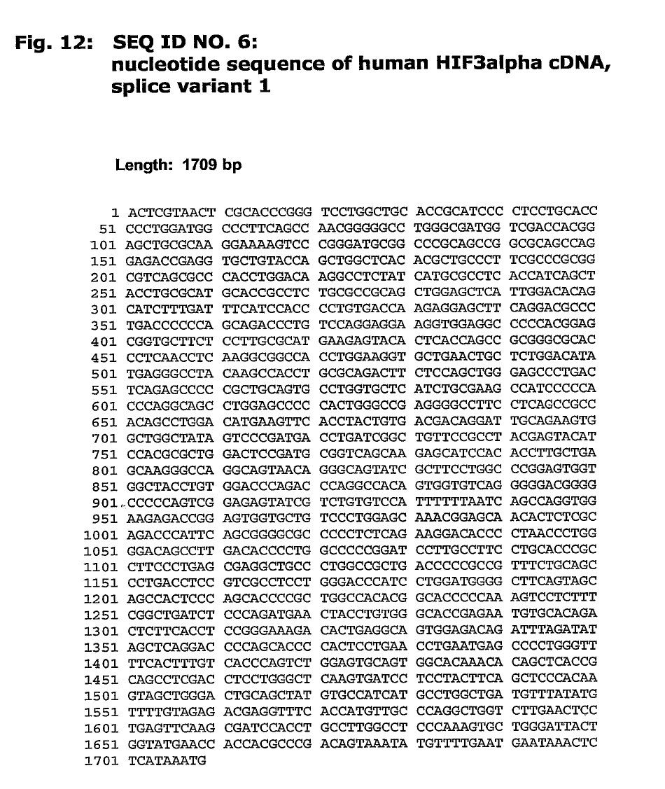

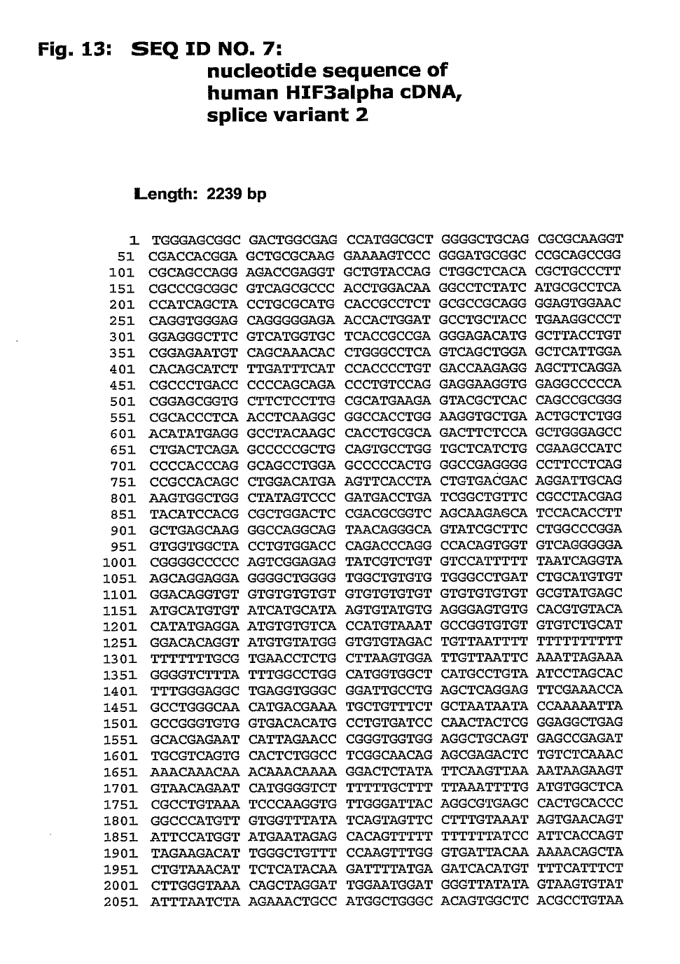

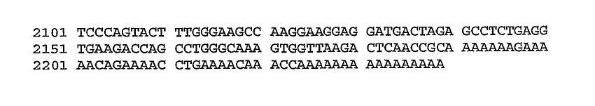

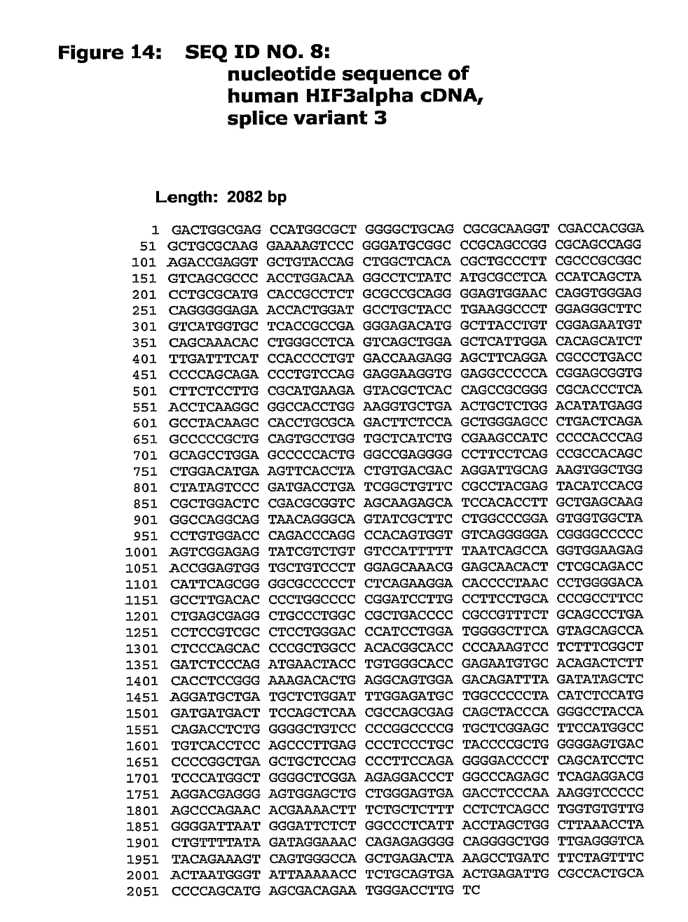

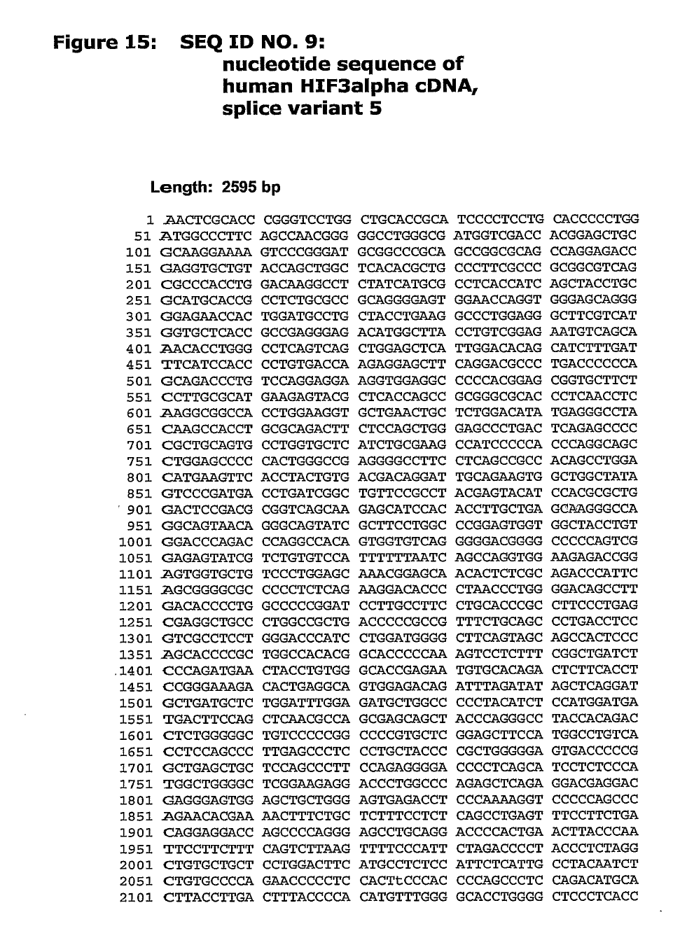

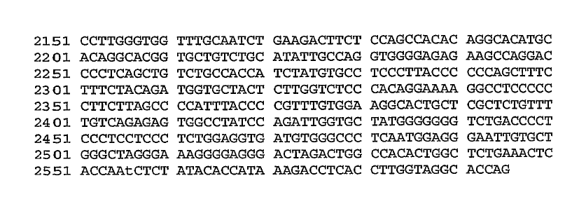

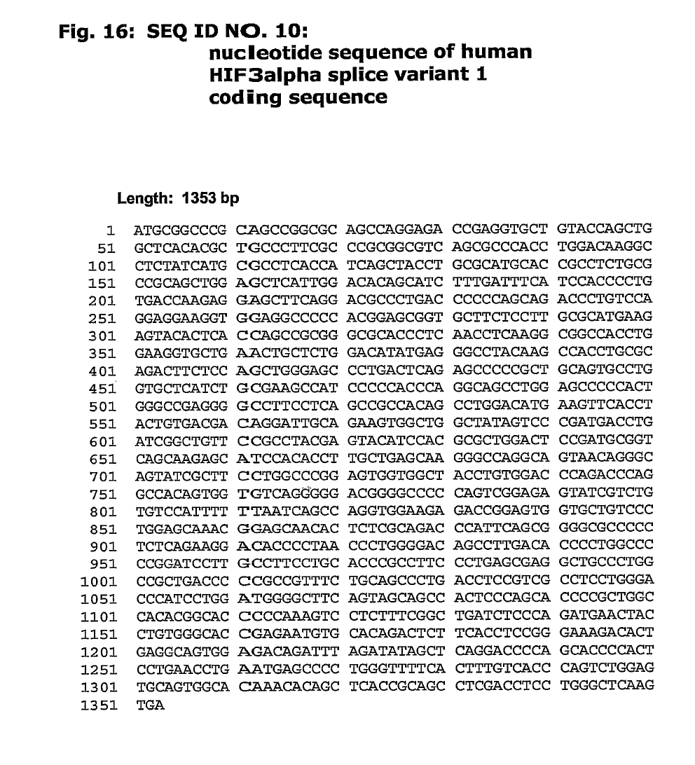

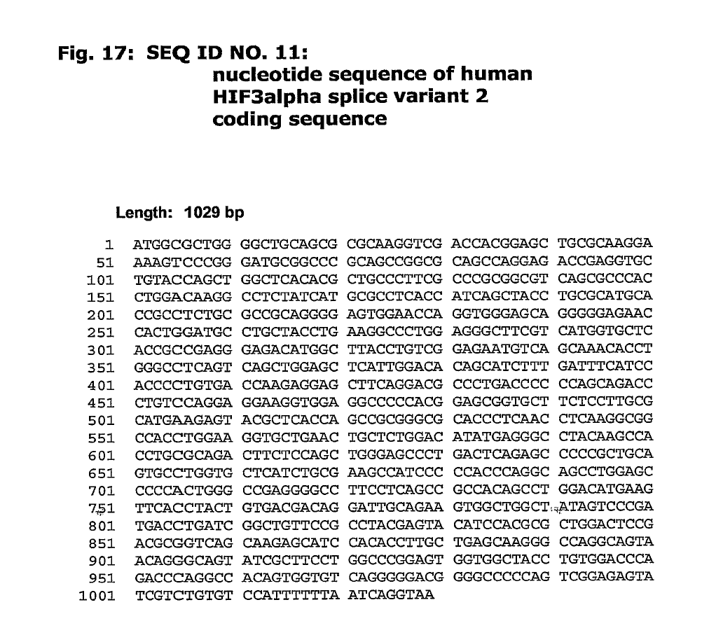

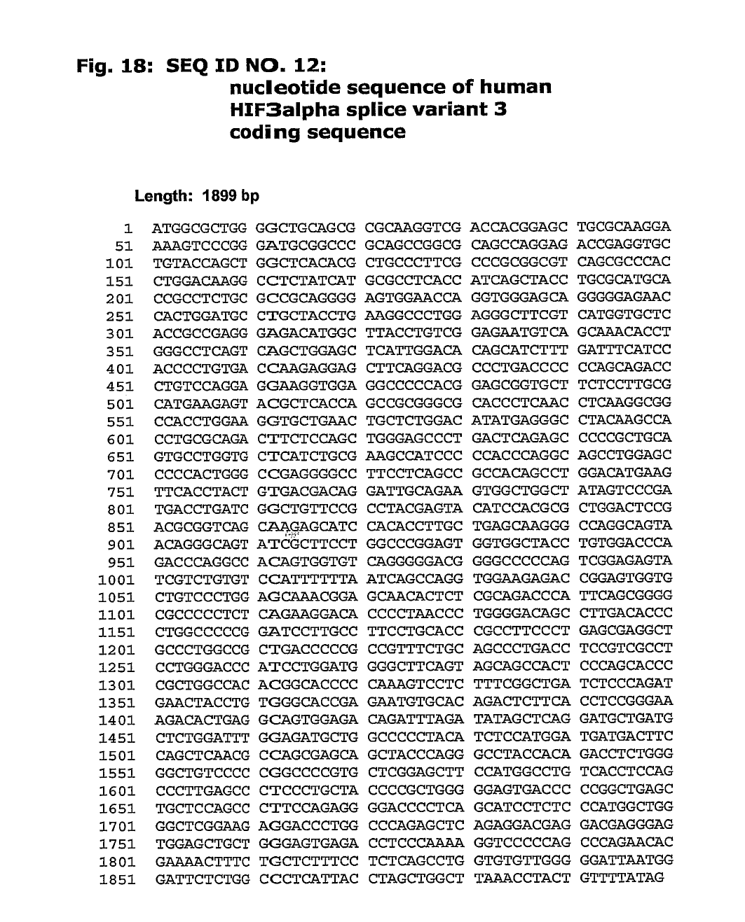

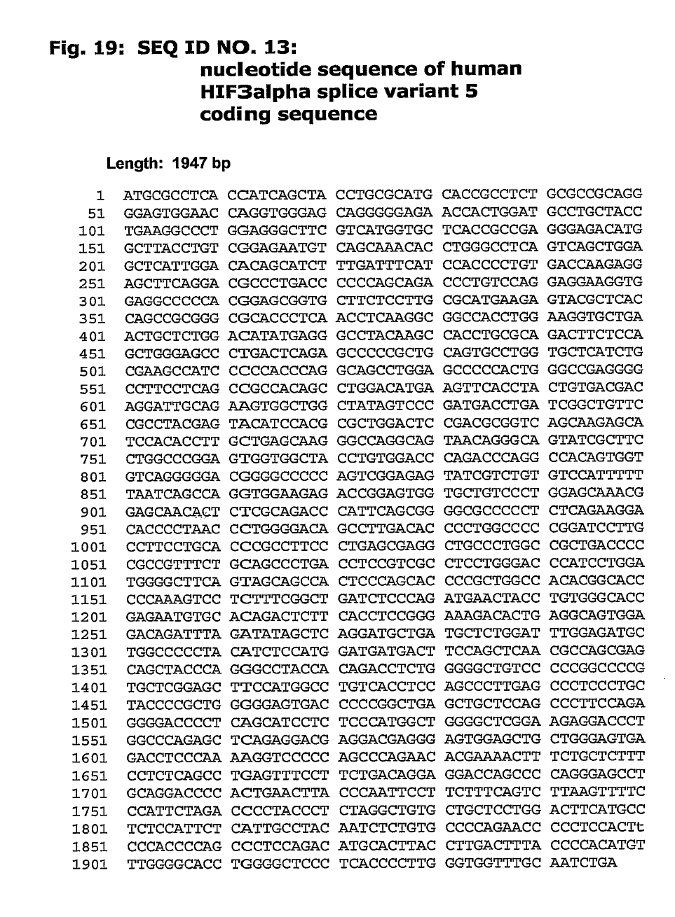

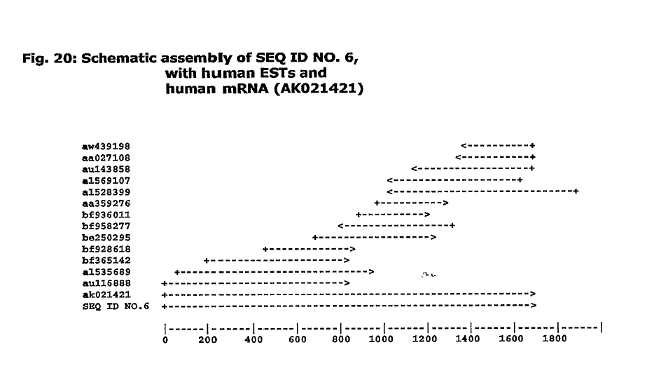

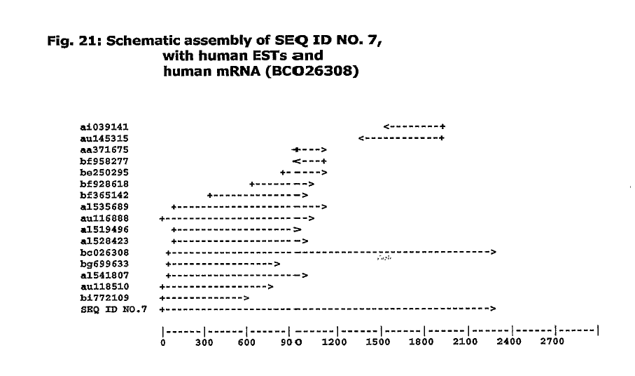

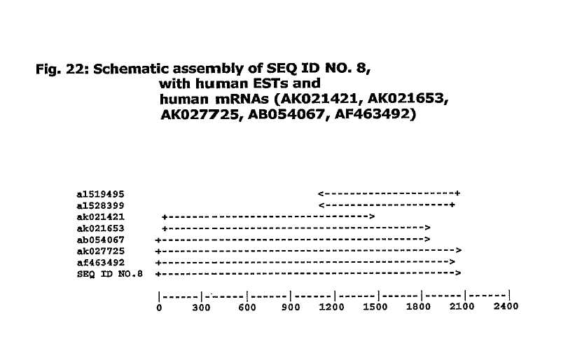

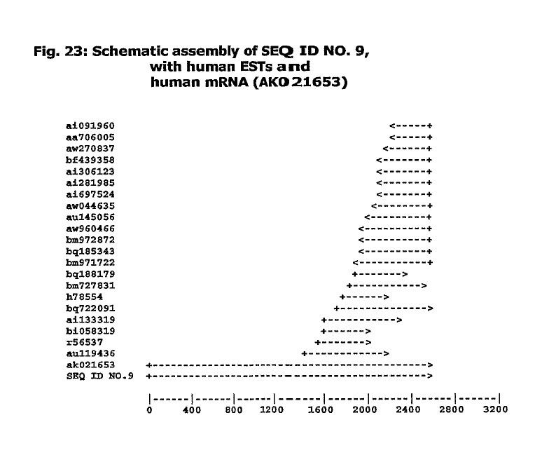

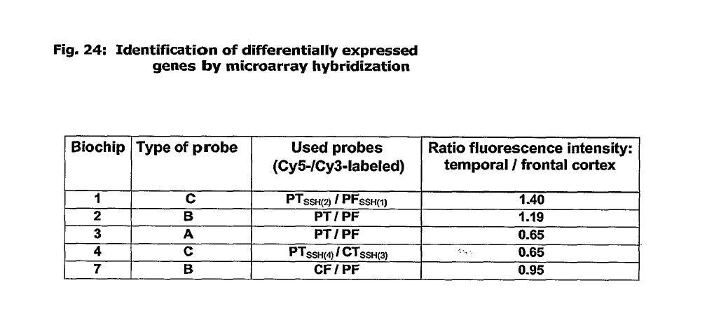

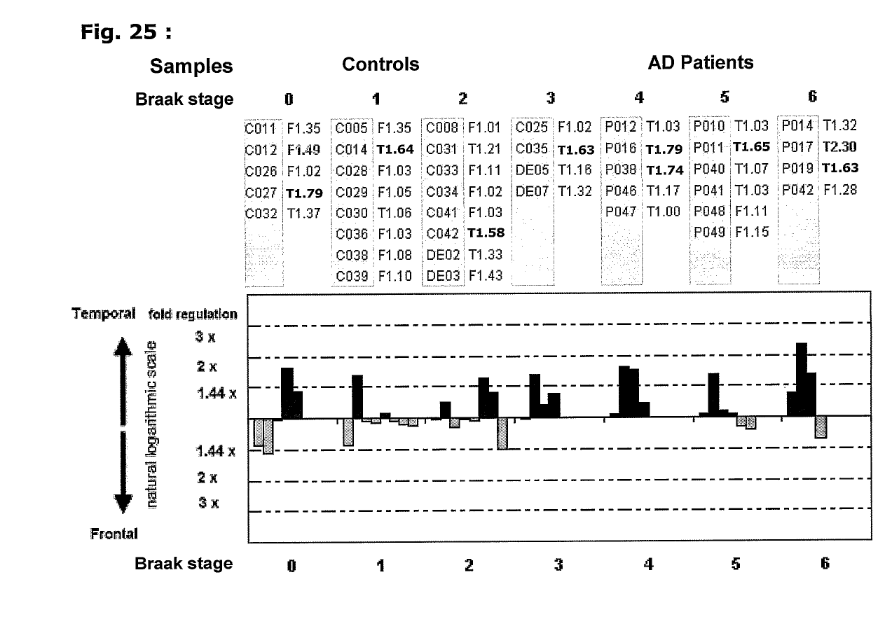

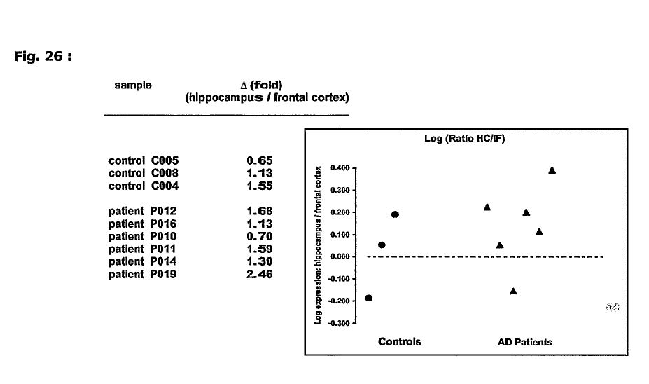

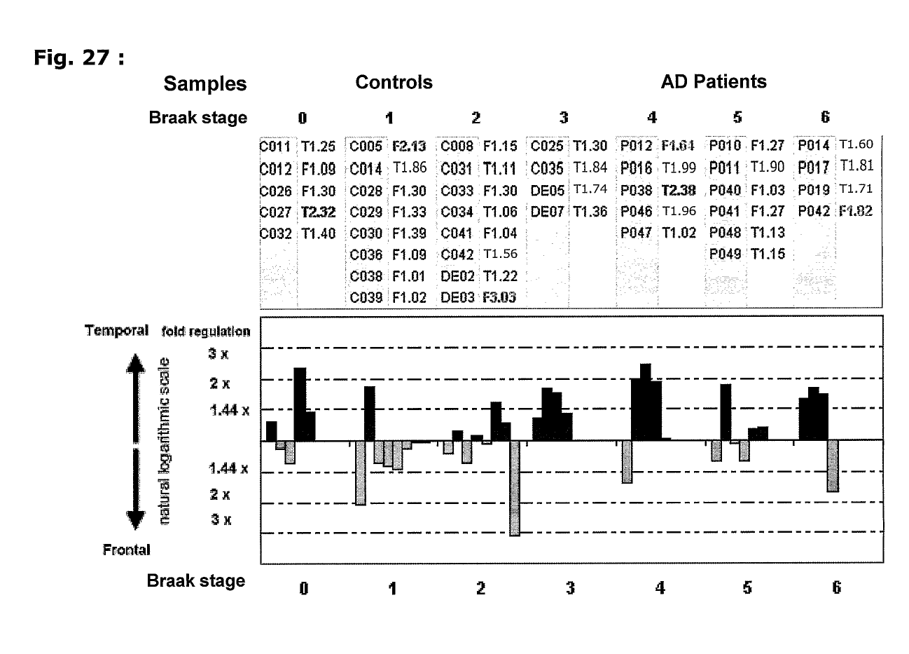

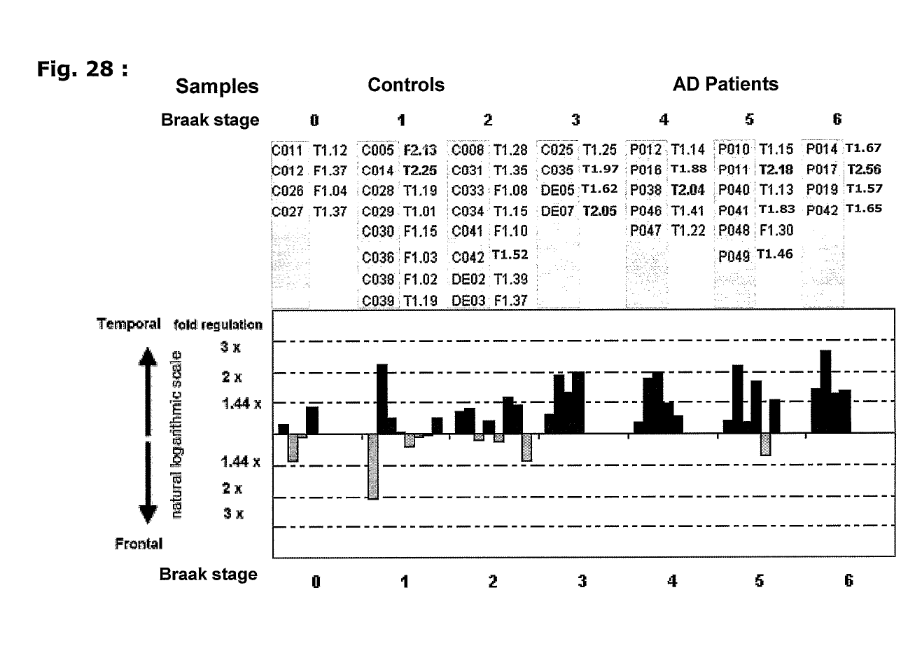

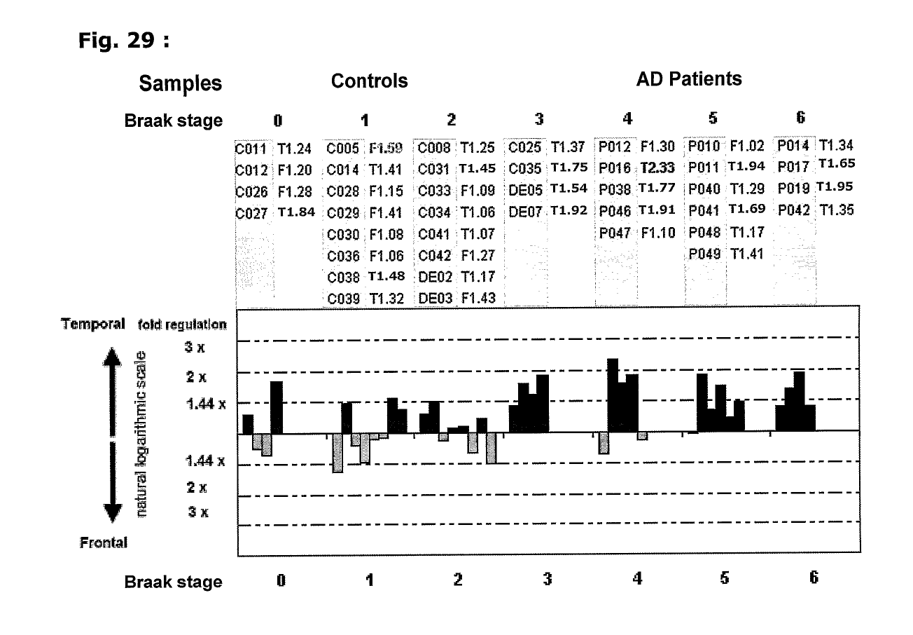

In a third step, the set of reference standard genes was analyzed in parallel to determine the mean average value of the AD patient to control person temporal cortex ratios, of the AD patient to control person frontal cortex ratios, and of the temporal to frontal ratios, and of the hippocampal to frontal ratios of AD patients and control persons, respectively, of expression levels of the reference standard genes for each individual brain sample. As cyclophilin B was analyzed in step 2 and step 3, and the ratio from one gene to another gene remained constant in different runs, it was possible to normalize the values for HIF3a, i.e. for the HIF3a splice variant 1, HIF3a splice variant 2, splice variant 3 and for the HIF3a splice variant 5, respectively, to the mean average value of the set of reference standard genes instead of normalizing to one single gene alone. The calculation was performed by dividing the respective ratios shown above by the deviation of cyclophilin B from the mean value of all housekeeping genes. The results of such quantitative RT-PCR analysis for the HIF3a gene and the respective calculated values for the HIF3a gene, i.e. for the HIF3a splice variant 1, HIF3a splice variant 2, HIF3a splice variant 3 and the HIF3a splice variant 5, are shown in FIGS. 1, 2 and 25, 26, in FIGS. 3 and 27, in FIGS. 4 and 28, in FIGS. 5 and 29, respectively.

2) Comparison of the mRNA expression between controls and AD patients. For this analysis it was proven that absolute values of real-time quantitative PCR (Lightcycler method) between different experiments at different time points are consistent enough to be used for quantitive comparisons without usage of calibrators. Cyclophilin was used as a standard for normalization in any of the qPCR experiments for more than 100 tissues. Between others it was found to be the most consistently expressed housekeeping gene in our normalization experiments. Therefore a proof of concept was done by using values that were generated for cyclophilin.

First analysis used cyclophilin values from qPCR experiments of frontal cortex and inferior temporal cortex tissues from three different donors. From each tissue the same cDNA preparation was used in all analyzed experiments. Within this analysis no normal distribution of values was achieved due to small number of data. Therefore the method of median and its 98% -conficence level was applied. This analysis revealed a middle deviation of 8.7% from the median for comparison of absolute values and a middle deviation of 6.6% from the median for relative comparison.

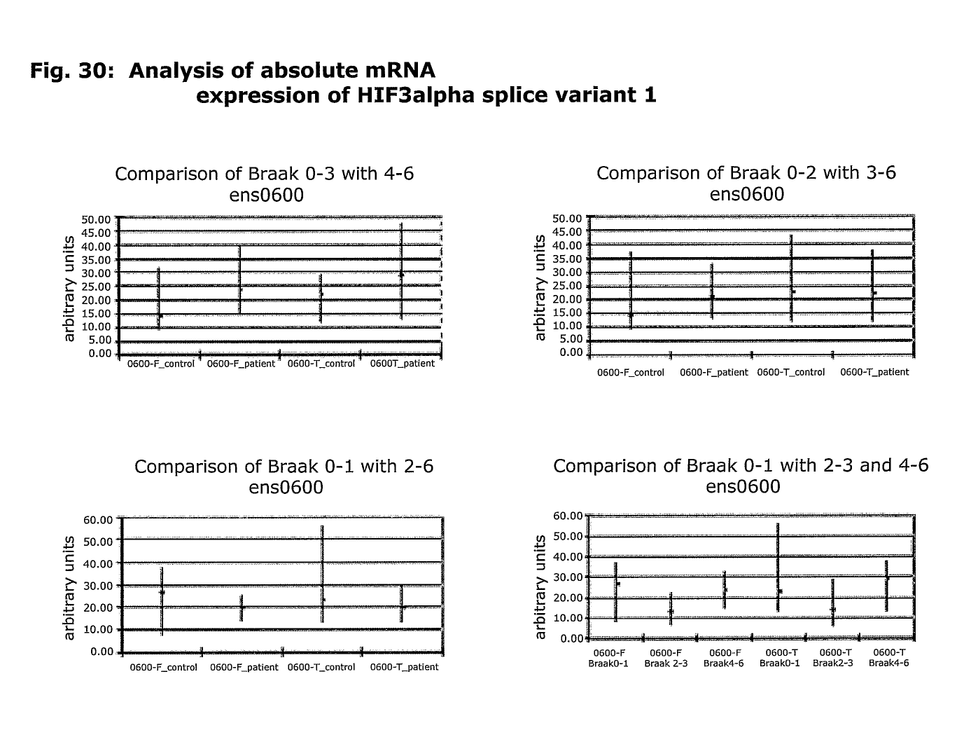

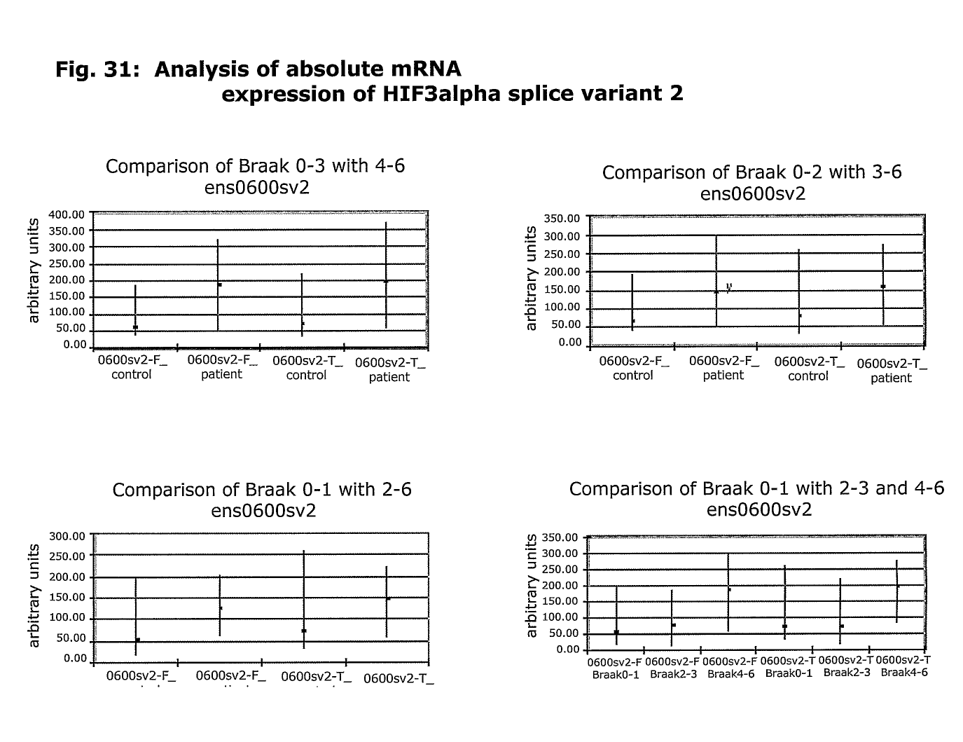

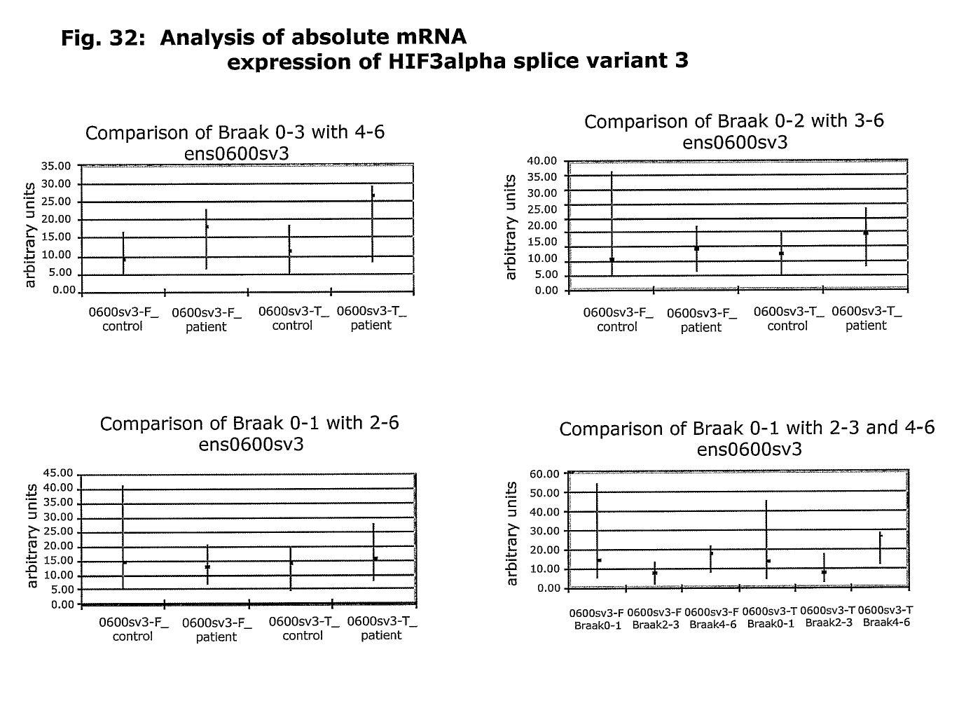

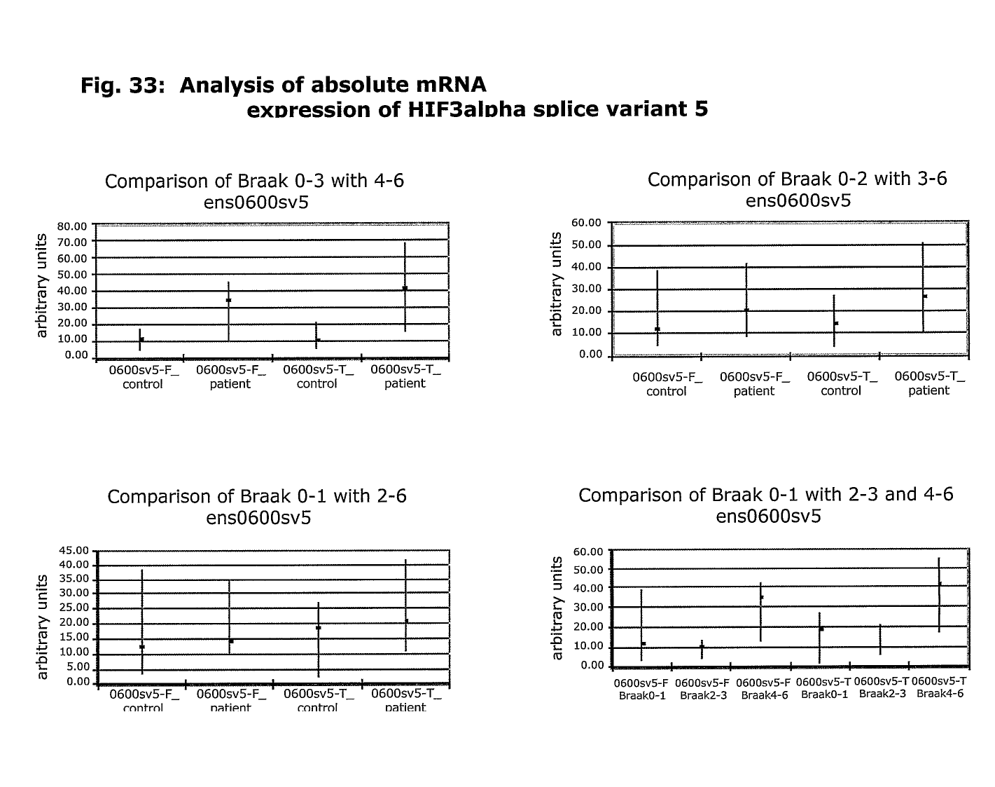

Second analysis used cyclophilin values from qPCR experiments of frontal cortex and inferior temporal cortex tissues from two different donors each, but different cDNA preparations from different time points were used. This analysis revealed a middle deviation of 29.2% from the median for comparison of absolute values and a middle deviation of 17.6% from the median for relative comparison. From this analysis it was concluded, that absolute values from qPCR experiments can be used, but the middle deviation from median should be taken into further considerations. A detailed analysis of absolute values for HIF3a was performed. Therefore, absolute levels of HIF3a were used after relative normalization with cyclophilin. The median as well as the 98%-c,confidence level was calculated for the control group (Braak 0-Braak 3) and the patient group (Braak 4-Braak 6), respectively. The same analysis was done redefining the control group (Braak 0-Braak 2) and the patient group (Braak 3-Braak 6) as well as redefining the control group (Braak 0-Braak 1) and the patient group (Braak 2-Braak 6). The latter analysis was aimed to identify early onset of mRNA expression differences between controls and AD patients. In another view of this analysis, three groups comprising Braak stages 0-1, Braak stages 2-3, and Braak stages 4-6, respectively, were compared to each other in order to identify tendencies of gene expression regulation as well as early onset differences. Said analysis as described above are shown in FIGS. 30, 31, 32, 33.

(ix) Immunoblotting:

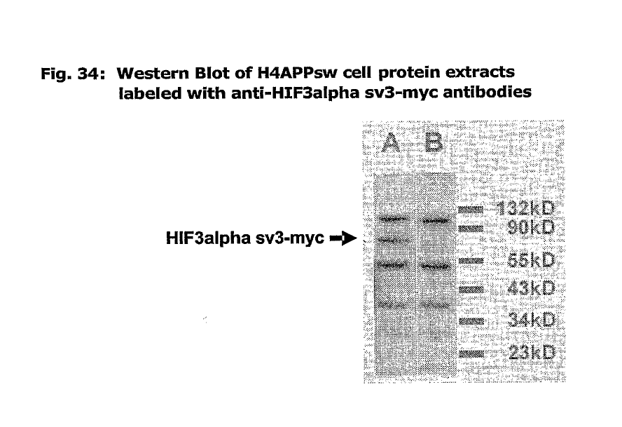

Total protein extract was obtained from H4APPsw cells expressing HIF3a sv3-myc by homogenization in 1 ml RIPA buffer (150 mM sodium chloride, 50 mM tris-HCl, pH7.4, 1 mM ethylenediamine-tetraacetic acid, 1 mM phenylmethylsulfonyl flouride, 1% Triton X-100, 1% sodium deoxycholic acid, 1% sodium dodecylsulfate, 5 μg/ml of aprotinin, 5 μg/ml of leupeptin) on ice. After centrifuging twice for 5 min at 3000 rpm at 4° C., the supernatant was diluted five-fold in SDS-loading buffer. Aliquots of 12 μl of the diluted sample were resolved by SDS-PAGE (8% polyacrylamide) and transferred to PVDF Western Blotting membranes (Boehringer Mannheim). The blots were probed with rabbit polyclonal anti-myc antibodies (MBL, 1:5000) followed by horseradish peroxidase-coupled goat anti-rabbit IgG antiserum (Santa Cruz sc-2030, diluted 1:5000) and developed with the ECL chemoluminescence detection kit (Amersham Pharmacia) (FIG. 34).

(x) Immunofluorescence Analysis (IF):

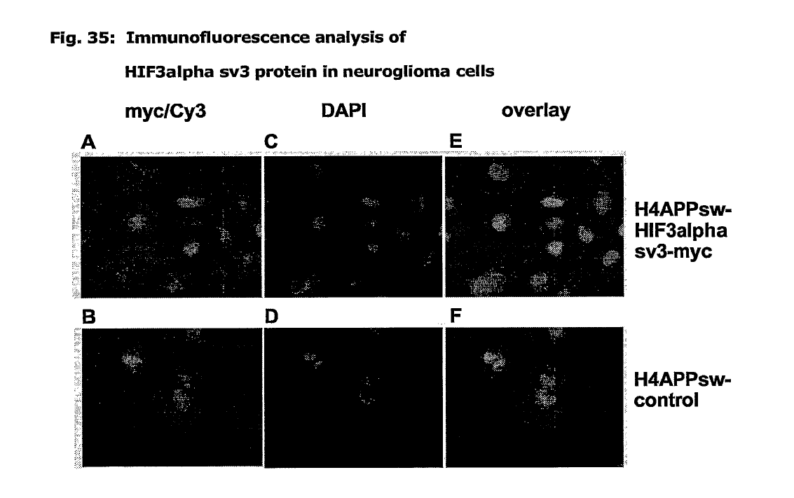

For the immunofluorescence staining of HIF3a protein in cells, a human neuroglioma cell line was used (H4 cells) which stably expresses the human APP695 isoform carrying the Swedish mutation (K670N, M671 L) (H4APPsw cells). The H4APPsw cells were transduced with a pFB-Neo vector (Stratagene, #217561) containing the coding sequence of HIF3a sv3 (HIF3a sv3 cds) (SEQ ID NO. 12, 1899 bp) and a myc-tag (pFB-Neo-HIF3a sv3 cds-myc, HIF3a sv3-myc vector, 9181 bp, EcoRI/Xhol) under the control of a strong CMV promotor. For the generation of the HIF3a sv3-myc vector, the HIF3a sv3 cds-myc sequence was introduced into the EcoRI/Xhol restriction sites of the multiple cloning site (MCS) of the pFB-Neo vector. For transduction of the H4APPsw cells with the HIF3a sv3-myc vector the retroviral expression system ViraPort from Stratagene was used. The myc-tagged HIF3a sv3 over-expressing cells (H4APPsw-HIF3a sv3-myc) were seeded onto glass cover slips in a 24 well plate (Nunc, Roskilde, Denmark; #143982) at a density of 5×10⁴ cells and incubated at 37° C. at 5% CO₂ over night. To fix the cells onto the cover slip, medium was removed and chilled methanol (−20° C.) was added. After an incubation period of 15 minutes at −20° C., methanol was removed and the fixed cells were blocked for 1 hour in blocking solution (200 μl PBS/5% BSA/3% goat serum) at room temperature. The first antibody (polyclonal anti-myc antibody, rabbit, 1:5000, MBL) and DAPI (DNA-stain, 0.05 μg/ml, 1:1000) in PBS/1% goat serum was added and incubated for 1 hour at room temperature. After removing the first antibody, the fixed cells were washed 3 times with PBS for 5 minutes. The second antibody (Cy3-conjugated anti-rabbit antibody, 1:1000, Amersham Pharmacia, Germany) was applied in blocking solution and incubated for 1 hour at room temperature. The cells were washed 3 times in PBS for 5 minutes. Coverslips were mounted onto microscope slides using Permafluor (Beckman Coulter) and stored over night at 4° C. to harden the mounting media. Cells were visualized using microscopic dark field epifluorescence and bright field phase contrast illumination conditions (IX81, Olympus Optical). Microscopic images (FIG. 35) were digitally captured with a PCO SensiCam and analysed using the appropriate software (AnalySiS, Olympus Optical).

(xi) Immunohistochemistry:

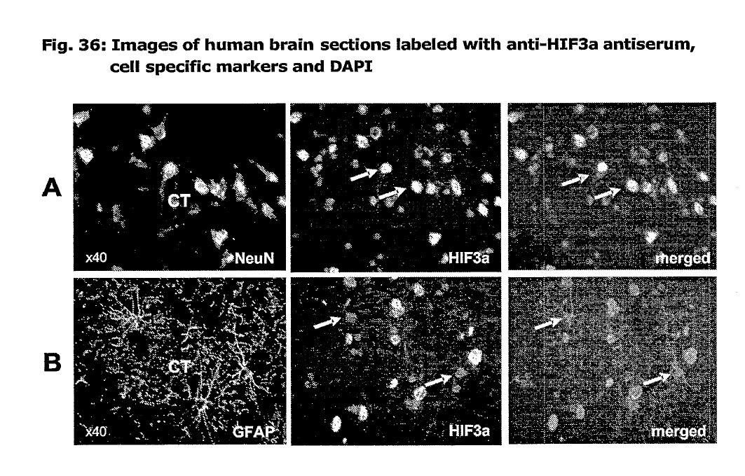

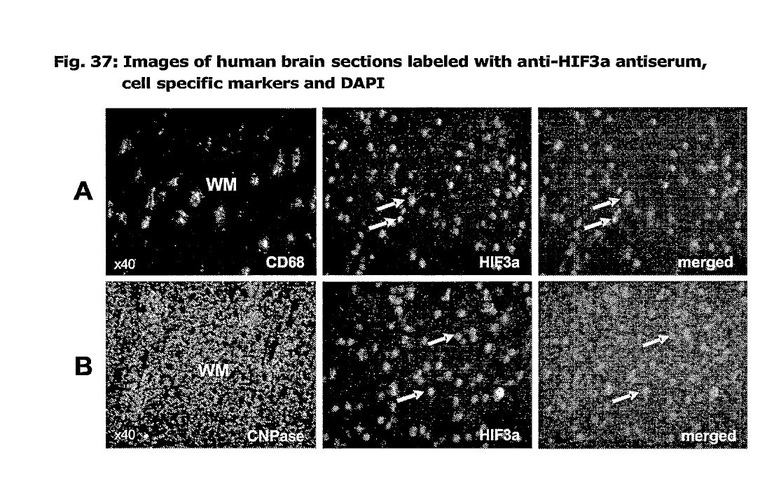

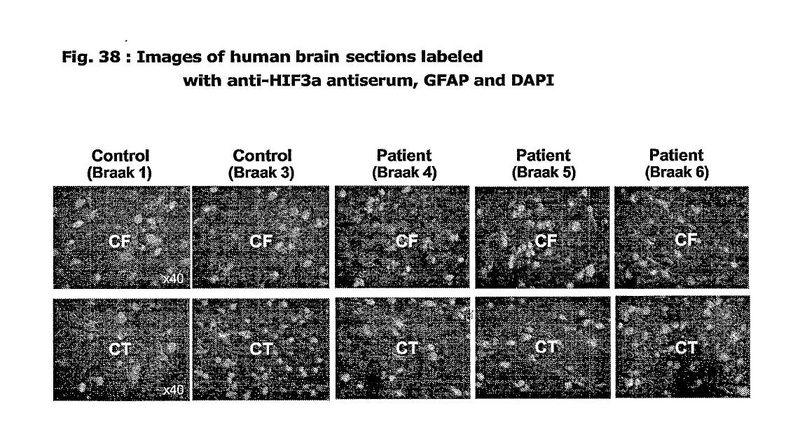

For immunofluorescence staining of HIF3a, respectively HIF3a svl, HIF3a sv3 and HIF3a sv5, in human brain, and for the comparison of AD-affected tissue with control tissues, post-mortem fresh-frozen frontal and temporal forebrain specimens from donors comprising patients with clinically diagnosed and neuropathologically confirmed Alzheimer's disease at various Braak stages (P016, P011, P017-Braak 4, 5, 6), as well as age-matched control individuals without Alzheimer (C029-Braak 1) and individuals which are at middle Braak stage level (C035-Braak 3), were cut at 14 μm thickness using a cryostat (Leica CM3050S). The tissue sections were air-dried and fixed in acetone for 10 min at room temperature. After washing in PBS, the sections were pre-incubated with blocking buffer (10% normal goat serum, 0.2% Triton X-100 in PBS) for 30 min and then incubated with affinity-purified rabbit polyclonal anti-HIF3a antiserum (1:40 diluted in blocking buffer; Davids Biotechnology; amino acids 290-304) overnight at 40° C. After rinsing three times in 0.1% Triton X-100/PBS, the sections were incubated with FITC-conjugated goat anti-rabbit IgG antiserum (Jackson/Dianova, No. 111-096-045, 1:150 diluted in 1% BSA/PBS) for 2 hours at room temperature and then again washed in PBS. Staining of the neuronal cells was performed by using a mouse monoclonal antibody against the neuronal specific marker NeuN (Chemicon, MAB377, dilution 1:400) and a secondary Cy3-conjugated goat anti-mouse antibody (Dianova, 115-166-062, dilution 1:600). Staining of the astrocytes was performed by using an antibody against the astrocyte-specific marker GFAP (Abcam, AB780b, dilution 1:300), staining of microglia was performed by using an antibody against the microglial specific marker CD68 (DAKO, Mo718, dilution 1:200) and staining against oligodendrocytes by using an antibody against the oligodendrocyte specific marker CNAPase (Sigma, C5P22, dilution 1:400). Staining of the nuclei was performed by incubation of the sections with 5 μM DAPI in PBS for 3 min. In order to block the autofluoresence of lipofuscin in human brain, the sections were treated with 1% Sudan Black B in 70% ethanol for 2-10 min at room temperature and then sequentially dipped in 70% ethanol, destilled water and PBS. The sections were coverslipped with ‘Vectashield’ mounting medium (Vector Laboratories, Burlingame, Calif.). Microscopic images were obtained using dark field epifluorescence and bright field phase contrast illumination conditions (IX81, Olympus Optical). Microscopic images were digitally captured with a PCO SensiCam and analyzed using the appropriate software (AnalySiS, Olympus Optical) (see FIGS. 36, 37 and 38).

(xii) Generation of Trans Genic Drosophila Melanogaster:

Human BACE transgenic flies and human HIF3a transgenic flies were generated according to Greeve et al. (Greeve et al., J. Neurosci. 2004, 24: 3899-3906) and as described in the present invention. A 1942 bp EcoRI/Xhol fragment of the HIF3a sv3 cDNA (SEQ ID NO. 8) containing the entire open reading frame of HIF3a sv3 (SEQ ID NO.12, SEQ ID NO.4) and fused in frame to a myc-tag (aa EQKLISEEDL) at the 3′ end was subcloned into the EcoRI/Xhol restriction sites of the vector pUAST downstream of the GAL4-binding sites UAS (Brand and Perrimon, Development 1993, 118: 401-15). P-element-mediated germline transformation was performed as described by Spradling and Rubin (Rubin and Spradling, Science 1982, 218: 348-53; Spradling and Rubin, Science 1982, 218: 341-7). Twentyeight independent human HIF3a sv3 transgenic fly lines were generated and three different lines were used for the analysis.

Human APP and Drosophila Presenilin transgenic flies, the UAS-APP695II and the UAS-DPsn-mutants (L235P), were kindly provided by R. Paro and E. Fortini (Fossgreen et al., Proc Natl Acad Sci USA 1998, 95: 13703-8; Ye and Fortini, J Cell Biol 1999, 146: 1351-64). The actin-GAL4 line was obtained from the Bloomington stock center. The gmr-GAL4 line from F. Pignoni was used to achieve the eye-specific expression of the transgenes.

Genetic crosses were set up on standard Drosophila culture medium at 25° C. Genotypes used were: w; UAS-hAPP₆₉₅, UAS-hBACE437/CyO; gmr-GAL4/Tm3-w; UAS-hAPP₆₉₅, UAS-hBACE437/CyO; gmr-GAL4,UAS-DPsnL235P/Tm3-w; UAS-HIF3a-sv3-myc#3-w; UAS-HIF3a-sv3-myc#4-w; UAS-HIF3a-sv3-myc#57.

For immunohistochemical and histological analysis the adult flies were immunostained and prepared according to the following methods. For immunostaining adult flies were fixed in 4% paraformaldehyde for 3 hours, washed in 1×PBS and transferred to 25% sucrose for an overnight incubation at 40° C. Flies were decapitated with a razor blade, and the heads were imbedded in Tissue Tek (Sakura) and snap frozen. 10 μm horizontal frozen sections were prepared on a cryostat (Leica CM3050S). Immunostaining was done with the Vectastain Elite kit (Vector Laboratories) according to the instructions of the manufacturer. The following primary antibodies were used: mouse monoclonal antibody 24B10 (alpha-chaoptin, 1:5) provided by the Developmental Studies Hybridoma Bank and a rabbit polyclonal anti-myc antibody (MBL, Medical and Biological Laboratories). Paraffin sections of adult heads and mass histology were done as described by Jager and Fischbach (Ashburner, Drosophila: A Laboratory Manual 1989, Cold Spring Harbor Laboratory Press, Cold Spring Harbor, NY: 254-259). For thioflavin S staining 5 μm paraffin sections were counterstained for 5 minutes in Mayers Hemalum (Sigma), rinsed for 10 minutes in tap water and stained for 3 minutes in 1% thioflavin S (Sigma) watery solution. Slides were rinsed in several changes of distilled water, incubated for 15 minutes in 1% acetic acid, rinsed in tap water and mounted in Vectashield mounting medium (Vector laboratories). Slides were analyzed under an Olympus BX51 fluorescence microscope (430 nm excitation, 550 nm emission).

For the protein analyses by western blotting, fly heads were homogenized in 1×PBS, 5 mM EDTA, 0.5% Triton X-100 and a protease-inhibitor mix Complete (Roche Applied Science). Equal amounts of protein were separated by 10% SDS-PAGE, transferred to Immobilon membranes (Millipore GmbH), blocked in 5% low fat milk for two hours at room temperature and incubated with a rabbit polyclonal anti-myc antibody (MBL, Medical and Biological Laboratories). Bound antibodies were detected with goat anti-mouse peroxidase conjugated secondary antibodies (Dianova). For immunoprecipitation fly heads were homogenized as described above and lysates were treated as described in the antibodies protocol guide from Clontech. The antibody mab 6E10 (alpha-Abeta1-16, Signet Pathology Systems) was used for immunoprecipitation. Samples were separated on 10-20% gradient Novex Tris-Tricine gels (Invitrogen) and blotted onto Protran BA 79 Cellulosenitrate membranes (0.1 μm, Schleicher/Schuell, Dassel, Germany). Detection of beta-amyloid was performed as described (Ida et al., J Biol Chem 1996, 271: 22908-14) using mab 6E10 and goat anti-mouse peroxidase conjugated secondary antibody (Dianova).

For detection of human HIF3a splice variant 3 expression in transgenic Drosophila a reverse transcriptase PCR reaction was performed using HIF3a splice variant 3 specific primers as described in example (viii). To calculate relative differences in expression levels of the three HIF3a sv3 transgenic fly lines that were used for all experiments a Drosophila housekeeping gene rp49 (ribosomal protein L32) was profiled in the same lightcycler run using the following rp49 specific primer pair: 5′-GAAGAAGCGCACCAAG GACT-3′ and 3′-TTGAATCCGGTGGGCAGCAT-5′.

To characterize the potential impact of human HIF3a sv3 expression on the neuropathology associated with amyloidogenic processing of human APP (beta-amyloid precursor protein, hAPP) in transgenic flies, HIF3a sv3 was co-expressed with hAPP and human BACE (Beta site APP cleaving enzyme, hBACE) in the adult retina by using the eye-specific GAL4 line gmr-GAL4.

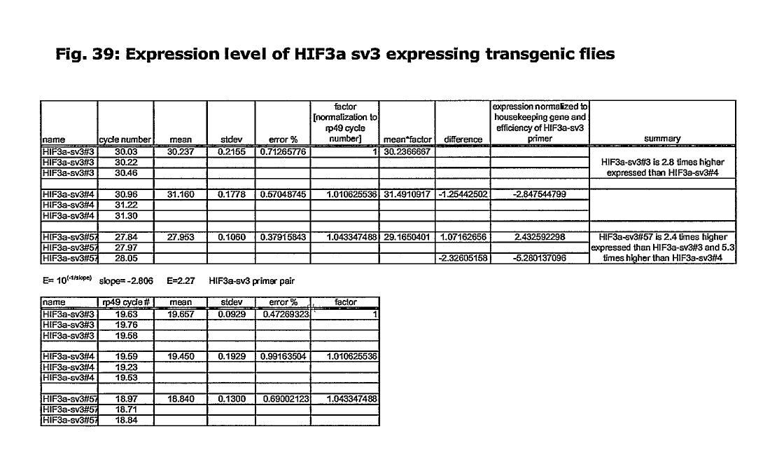

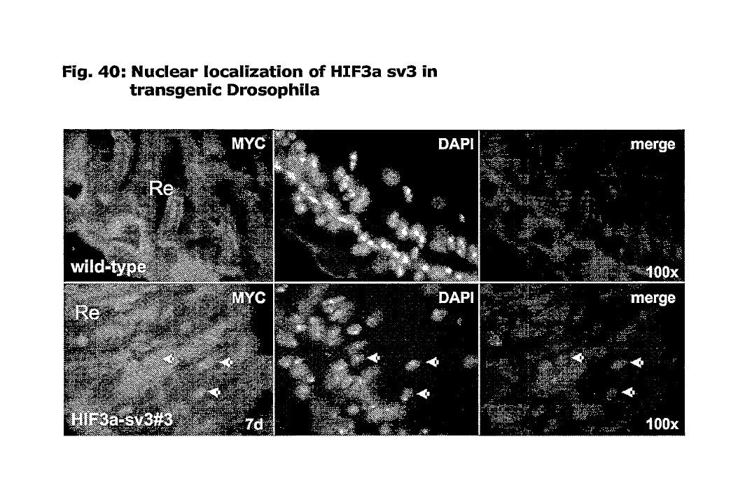

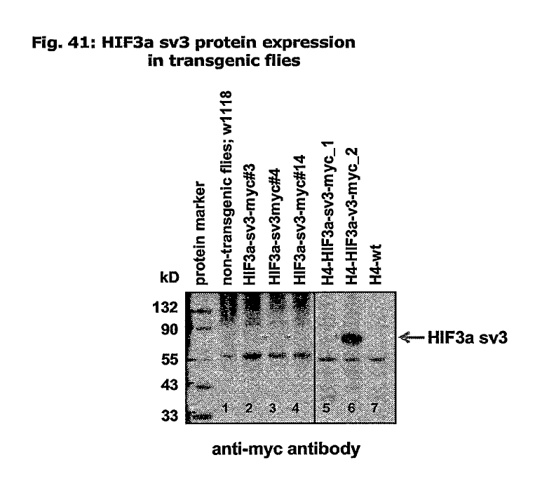

Transgenic expression of HIF3a sv3 under the control of gmr-GAL4 was confirmed by RT-PCR using HIF3a sv3 specific primers (see Example viii). Three different transgenic fly lines were used (HIF3a-sv3-myc#3, #4 and #57). Relative differences in the expression efficiency (FIG. 39) were calculated according to the cycle number of the HIF3a sv3 primer pair and normalized to cycle numbers of the rp49 gene of Drosophila (ribosomal protein L32). Based on this calculation HIF3a sv3 transgenic fly line #57 is 2.4 times higher expressed than fly line #3 and 5.3 times stronger than fly line #4 (FIG. 39). Transgenic expression of human HIF3a sv3 under the control of gmr-GAL4 results in a nuclear localization of the protein in the retina of Drosophila (FIG. 40, arrowheads). On Western Blots a polyclonal anti-myc antibody (MBL) detects a protein with the expected molecular weight of HIF3a sv3 (70 kD, FIG. 41 arrow) which is absent from protein extracts of non-transgenic control flies. Interestingly, HIF3a sv3 expression is hardly detectable on protein level in other transgenic flies (FIG. 41, lanes 2, 3 and 4, red asterisk) as well as H4-APPsw cells stably transfected with a human HIF3a sv3-myc construct (lane 5, as shown in example (X)) which suggests protein degradation or low expression of transgenic HIF3a sv3-myc under normoxia conditions in Drosophila and human H4-neuroglioma cells.

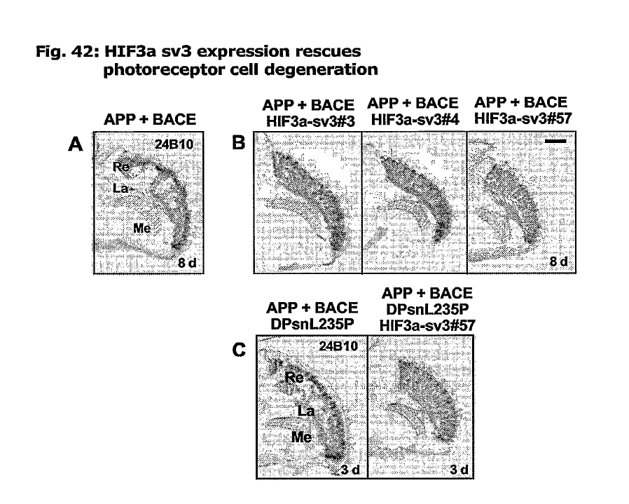

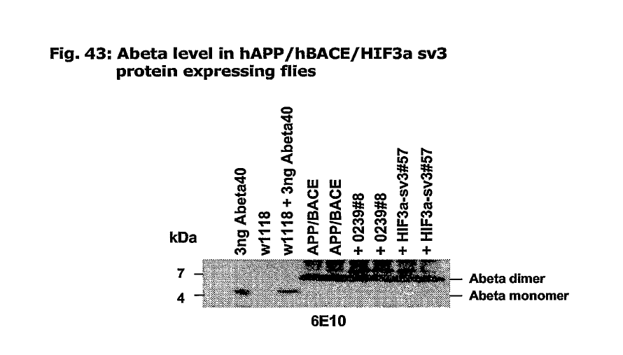

Expression of hAPP and hBACE in the adult retina of Drosophila leads to age-dependent degeneration of photoreceptor cells (FIG. 42A, Greeve, I. et al., J. Neurosci. 2004, 24: 3899-3906). Co-expression of HIF3a sv3 rescues photoreceptor cell degeneration in age-matched 8 days old male and female flies (FIG. 42B) as demonstrated on cryostat sections through the brain and complex eye of adult flies that were stained with a photoreceptor cell specific monoclonal antibody 24B10 (FIG. 42). Photoreceptor cell degeneration is accelerated in flies co-expressing a mutated form of Presenilin (PsnL235P, FIG. 43 and Greeve, l. et al., J. Neurosci. 2004, 24: 3899-3906). Rescue of photoreceptor cell degeneration is even more evident in 3 day old male and female flies co-expressing HIF3a-sv3#57 with hAPP, hBACE and DPsnL235P when compared to age-matched control flies (FIG. 42C).

To further characterize the neuroprotective effect of HIF3a sv3 on photoreceptor cell degeneration in hAPP/hBACE expressing flies, the amyloidogenic processing of hAPP in triple transgenic flies was investigated and the co-expression of HIF3a-sv3#57 was examined which shows the highest impact on the neuropathology associated with APP processing in the model system. Abeta level are not changed in control flies (hAPP/hBACE) when compared to flies co-expressing hAPP/hBACE and HIF3a sv3 as demonstrated on Western blots of immunoprecipitated Abeta-peptides that were probed with the Abeta N-terminal specific antibody 6E10 (FIG. 43).

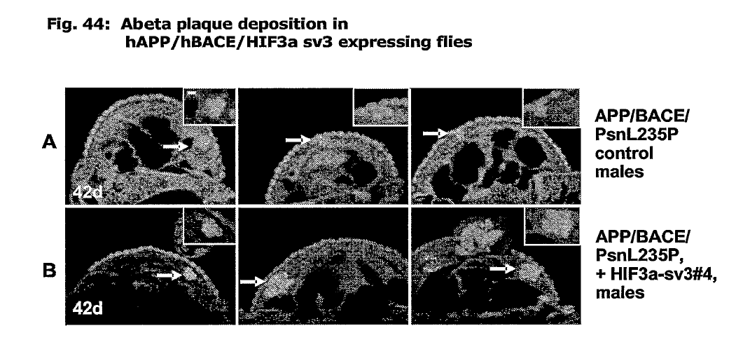

Further, the onset of Thioflavin S positive amyloid plaques in flies expressing hAPP/hBACE/DPsnL235P was investigated (FIG. 44 upper panel) and compared to flies expressing hAPP/hBACE/DPsnL235P and HIF3a-sv3#4 (FIG. 44 lower panel). FIG. 44 demonstrates no difference in the time of onset of plaque deposition between control flies and flies co-expressing HIF3a-sv3#4.

In summary, the strong neuroprotective effect of HIF3a sv3 is independent on altered amyloidogenic processing of hAPP or amyloid plaque formation in the described invertebrate model system of Alzheimer's disease.

1. A method of diagnosing or prognosticating a neurodegenerative disease in a subject, or determining whether a subject is at increased risk of developing said disease, comprising:
determining a level and/or an activity of
(i) a transcription product of a gene coding for HIF3a, and/or
(ii) a translation product of a gene coding for HIF3a, and/or
(iii) a fragment, or derivative, or variant of said transcription and/or translation product,
in a sample obtained from said subject and comparing said level or said activity, or both said level and said activity of said transcription product and/or said translation product to a reference value representing a known disease status and/or to a reference value representing a known health status, and said level and/or said activity is varied or altered compared to a reference value representing a known health status, and/or is similar or equal to a reference value representing a known disease status, thereby diagnosing or prognosticating said neurodegenerative disease in said subject, or determining whether said subject is at increased risk of developing said neurodegenerative disease.

2. The method according to claim 1 wherein said neurodegenerative disease is Alzheimer's disease.

3. A kit for diagnosing or prognosticating a neurodegenerative disease in a subject, or determining the propensity or predisposition, or the risk of a subject to develop such a disease, said kit comprising:
at least one reagent which is selected from the group consisting of
(i) reagents that selectively detect a transcription product of a gene coding for HIF3a and
(ii) reagents that selectively detect a translation product of a gene coding for HIF3a;
whereby the diagnosis or prognosis or determination of the risk to develop said neurodegenerative disease is determined by the steps of
(a) detecting in a sample obtained from said subject a level, or an activity, or both said level and said activity of a transcription product and/or of a translation product of a gene coding for HIF3a, and
(b) comparing said level or activity, or both said level and said activity of a transcription product and/or of a translation product of a gene coding for HIF3a to a reference value representing a known health status and/or to a reference value representing a known disease status, and said level, or activity, or both said level and said activity, of said transcription product and/or said translation product is varied compared to a reference value representing a known health status, and/or is similar or equal to a reference value representing a known disease status.

4. A genetically altered non-human animal comprising a non-native gene sequence coding for HIF3a, or a fragment, or a derivative, or a variant thereof.

5. The genetically altered non-human animal according to claim 4 wherein said non-human animal is a mammal or an invertebrate animal.

6. The genetically altered non-human animal according to claim 4, wherein the expression of said genetic alteration results in said non-human animal exhibiting a predisposition to developing symptoms, and/or displaying symptoms of neuropathology similar to a neurodegenerative disease.

7. The genetically altered non-human animal according to claim 4, wherein the expression of said genetic alteration results in said non-human animal which has a reduced risk of developing symptoms similar to a neurodegenerative disease, and/or which shows a reduction of said symptoms and/or which has no symptoms due to an effect caused by the expression of the gene used to genetically alter said non-human animal.

8. A method of developing diagnostics and therapeutics to treat neurodegenerative diseases, comprising screening, testing, or validating compounds, agents, and modulators using the genetically altered non-human animal according to claim 4.

9. A modulator of an activity and/or of a level of at least one substance which is selected from the group consisting of
(i) a gene coding for HIF3a,
(ii) a transcription product of a gene coding for HIF3a,
(iii) a translation product of a gene coding for HIF3a, and
(iv) a fragment, or derivative, or variant of (i) to (iii).

10. A method for screening for a modulator of neurodegenerative diseases, or related diseases or disorders of one or more substances selected from the group consisting of
(i) a gene coding for HIF3a,
(ii) a transcription product of a gene coding for HIF3a,
(iii) a translation product of a gene coding for HIF3a, and
(iv) a fragment, or derivative, or variant of (i) to (iii), said method comprising:
(a) contacting a cell with a test compound;
(b) measuring the activity and/or level of one or more substances recited in (i) to (iv);
(c) measuring the activity and/or level of one or more substances recited in (i) to (iv) in a control cell not contacted with said test compound; and
(d) comparing the levels and/or activities of the substance in the cells of step (b) and (c), wherein an alteration in the activity and/or level of substances in the contacted cells indicates that the test compound is a modulator of said diseases or disorders.

11. A method of screening for a modulator of neurodegenerative diseases, or related diseases or disorders of one or more substances selected from the group consisting of
(i) a gene coding for HIF3a,
(ii) a transcription product of a gene coding for HIF3a,
(iii) a translation product of a gene coding for HIF3a, and
(iv) a fragment, or derivative, or variant of (i) to (iii), said method comprising:
(a) administering a test compound to a test animal which is predisposed to developing or has already developed symptoms of a neurodegenerative disease or related diseases or disorders in respect of the substances recited in (i) to (iv);
(b) measuring the activity and/or level of one or more substances recited in (i) to (iv);
(c) measuring the activity and/or level of one or more substances recited in (i) to (iv) in a matched control animal which is predisposed to developing or has already developed symptoms of a neurodegenerative disease or related diseases or disorders in respect to the substances recited in (i) to (iv) and to which animal no such test compound has been administered;
(d) comparing the activity and/or level of the substance in the animals of step (b) and (c), wherein an alteration in the activity and/or level of substances in the test animal indicates that the test compound is a modulator of said diseases or disorders.

12. The method according to claim 11 wherein said test animal and/or said control animal is a genetically altered non-human animal which expresses the gene coding for HIF3a, or a fragment, or a derivative, or a variant thereof, under the control of a transcriptional control element which is not the native HIF3a gene transcriptional control element.

13. An assay for testing a compound or a plurality of compounds to determine the degree of binding of said compounds to a HIF3a translation product, or to a fragment, or derivative, or variant thereof, said assay comprising the steps of:
(i) adding a liquid suspension of said HIF3a translation product, or a fragment, or derivative, or variant thereof, to a plurality of containers;
(ii) adding a detectable compound or a plurality of detectable compounds to be screened for said binding to said plurality of containers;
(iii) incubating said HIF3a translation product, or said fragment, or derivative, or variant thereof, and said detectable compound or compounds;
(iv) measuring amounts of detectable compound or compounds associated with said HIF3 a translation product, or with said fragment, or derivative, or variant thereof; and
(v) determining the degree of binding by one or more of said compounds to said HIF3a translation product, or said fragment, or derivative, or variant thereof.

14. The method of claim 1, comprising determining a level and/or an activity of protein molecules of SEQ ID NO. 2, SEQ ID NO. 3, SEQ ID NO. 4, or SEQ ID NO. 5, said protein molecules being translation products of the gene coding for HIF3a, or fragments, or derivatives, or variants thereof.

15. The method of claim 10, wherein said screening is for a modulator of protein molecules of SEQ ID NO. 2, SEQ ID NO. 3, SEQ ID NO. 4, or SEQ ID NO. 5, said protein molecules being translation products of the gene coding for HIF3a, or fragments, or derivatives, or variants thereof, wherein said modulator is a reagent or compound for preventing, or treating, or ameliorating a neurodegenerative disease.

16. A method for detecting the pathological state of a cell in a sample obtained from a subject, comprising immunocytochemical staining of said cell with an antibody specifically immunoreactive with an immunogen, wherein said immunogen is a translation product of a gene coding for HIF3a, SEQ ID NO. 2, SEQ ID NO. 3, SEQ ID NO. 4, SEQ ID NO. 5, or a fragment, or derivative, or variant thereof, wherein an altered degree of staining, or an altered staining pattern in said cell compared to a cell representing a known health status indicates a pathological state of said cell which relates to a neurodegenerative disease.

17. The kit of claim 3, wherein said neurodegenerative disease is Alzheimer's disease.

18. The kit of claim 3, wherein said translation product is a protein molecule of SEQ ID NO. 2, SEQ ID NO. 3, SEQ ID NO. 4, or SEQ ID NO. 5, said protein molecule being a translation product of the gene coding for HIF3a, or a fragment, or derivative, or variant thereof.

19. The genetically altered non-human animal according to claim 5 wherein said mammal is a rodent, mouse, rat or guinea pig and said invertebrate animal is an insect or a fly.

20. The genetically altered non-human animal according to claim 19 wherein said fly is Drosophila melanogaster.

21. The genetically altered non-human animal according to claim 6, wherein said neurodegenerative disease is Alzheimer's disease.

22. The genetically altered non-human animal according to claim 7, wherein said neurodegenerative disease is Alzheimer's disease.

23. The method of claim 8, wherein said neurodegenerative disease is Alzheimer's disease.

24. The method of claim 10, wherein said neurodegenerative disease is Alzheimer's disease.

25. The method of claim 11, wherein said neurodegenerative disease is Alzheimer's disease.

26. The method of claim 11, wherein said screening is for a modulator of protein molecules of SEQ ID NO. 2, SEQ ID NO. 3, SEQ ID NO. 4, or SEQ ID NO. 5, said protein molecules being translation products of the gene coding for HIF3a, or fragments, or derivatives, or variants thereof, wherein said modulator is a reagent or compound for preventing, or treating, or ameliorating a neurodegenerative disease.

27. The assay of claim 13, wherein said detectable compound is a fluorescently detectable compound.

28. The method of claim 16, wherein said neurodegenerative disease is Alzheimer's disease.


SEQUENCE LISTING (31 sequences)

SEQ ID NO: 1 - DNA, 289 bp, Artificial Sequence
  Description of Artificial SequenceHIF3a cDNA fragment
catttatgagagtttattcattcaaaacatatttactgtcgggcgtggtggttcataccagtaatcccagcactttgggaggccaaggcaggtggatcgcttgaactcaggagttcaagaccagcctgggcaacatggtggaacttcgtctctacaaaacatataaacatcagccaggcatgatggcacatagctgcagtcccagctacttgtgggagctgaagtaggaggatcacttgagcccaggaggtcgaggctgtggtgagctgtgtttgtgccactgcactcc

SEQ ID NO: 2 - protein, 450 aa, Homo sapiens
MRPAAGAARRPRCCTSWLTRCPSPAASAPTWTRPLSCASPSATCACTASAPQLELIGHSIFDFIHPCDQEELQDALTPQQTLSRRKVEAPTERCFSLRMKSTLTSRGRTLNLKAATWKVLNCSGHMRAYKPPAQTSPAGSPDSEPPLQCLVLICEAIPHPGSLEPPLGRGAFLSRHSLDMKFTYCDDRIAEVAGYSPDDLIGCSAYEYIHALDSDAVSKSIHTLLSKGQAVTGQYRFLARSGGYLWTQTQATVVSGGRGPQSESIVCVHFLISQVEETGVVLSLEQTEQHSRRPIQRGAPSQKDTPNPGDSLDTPGPRILAFLHPPSLSEAALAADPRRFCSPDLRRLLGPILDGASVAATPSTPLATRHPQSPLSADLPDELPVGTENVHRLFTSGKDTEAVETDLDIAQDPSTPLLNLNEPLGFHFVTQSGVQWHKHSSPQPRPPGLK

SEQ ID NO: 3 - protein, 342 aa, Homo sapiens
MALGLQRARSTTELRKEKSRDAARSRRSQETEVLYQLAHTLPFARGVSAHLDKASIMRLTISYLRMHRLCAAGEWNQVGAGGEPLDACYLKALEGFVMVLTAEGDMAYLSENVSKHLGLSQLELIGHSIFDFIHPCDQEELQDALTPQQTLSRRKVEAPTERCFSLRMKSTLTSRGRTLNLKAATWKVLNCSGHMRAYKPPAQTSPAGSPDSEPPLQCLVLICEAIPHPGSLEPPLGRGAFLSRHSLDMKFTYCDDRIAEVAGYSPDDLIGCSAYEYIHALDSDAVSKSIHTLLSKGQAVTGQYRFLARSGGYLWTQTQATVVSGGRGPQSESIVCVHFLIR

SEQ ID NO: 4 - protein, 632 aa, Homo sapiens
MALGLQRARSTTELRKEKSRDAARSRRSQETEVLYQLAHTLPFARGVSAHLDKASIMRLTISYLRMHRLCAAGEWNQVGAGGEPLDACYLKALEGFVMVLTAEGDMAYLSENVSKHLGLSQLELIGHSIFDFIHPCDQEELQDALTPQQTLSRRKVEAPTERCFSLRMKSTLTSRGRTLNLKAATWKVLNCSGHMRAYKPPAQTSPAGSPDSEPPLQCLVLICEAIPHPGSLEPPLGRGAFLSRHSLDMKFTYCDDRIAEVAGYSPDDLIGCSAYEYIHALDSDAVSKSIHTLLSKGQAVTGQYRFLARSGGYLWTQTQATVVSGGRGPQSESIVCVHFLISQVEETGVVLSLEQTEQHSRRPIQRGAPSQKDTPNPGDSLDTPGPRILAFLHPPSLSEAALAADPRRFCSPDLRRLLGPILDGASVAATPSTPLATRHPQSPLSADLPDELPVGTENVHRLFTSGKDTEAVETDLDIAQDADALDLEMLAPYISMDDDFQLNASEQLPRAYHRPLGAVPRPRARSFHGLSPPALEPSLLPRWGSDPRLSCSSPSRGDPSASSPMAGARKRTLAQSSEDEDEGVELLGVRPPKRSPSPEHENFLLFPLSLVCWGINGILWPSLPSWLKPTVL

SEQ ID NO: 5 - protein, 648 aa, Homo sapiens
MRLTISYLRMHRLCAAGEWNQVGAGGEPLDACYLKALEGFVMVLTAEGDMAYLSENVSKHLGLSQLELIGHSIFDFIHPCDQEELQDALTPQQTLSRRKVEAPTERCFSLRMKSTLTSRGRTLNLKAATWKVLNCSGHMRAYKPPAQTSPAGSPDSEPPLQCLVLICEAIPHPGSLEPPLGRGAFLSRHSLDMKFTYCDDRIAEVAGYSPDDLIGCSAYEYIHALDSDAVSKSIHTLLSKGQAVTGQYRFLARSGGYLWTQTQATVVSGGRGPQSESIVCVHFLISQVEETGVVLSLEQTEQHSRRPIQRGAPSQKDTPNPGDSLDTPGPRILAFLHPPSLSEAALAADPRRFCSPDLRRLLGPILDGASVAATPSTPLATRHPQSPLSADLPDELPVGTENVHRLFTSGKDTEAVETDLDIAQDADALDLEMLAPYISMDDDFQLNASEQLPRAYHRPLGAVPRPRARSFHGLSPPALEPSLLPRWGSDPRLSCSSPSRGDPSASSPMAGARKRTLAQSSEDEDEGVELLGVRPPKRSPSPEHENFLLFPLSLSFLLTGGPAPGSLQDPTELTQFLLSVLSFPILDPYPLGCAAPGLHASPFSLPTISVPQNPLHFPPQPSRHALTLTLPHMFGAPGAPSPLGWFAI

SEQ ID NO: 6 - DNA, 1709 bp, Artificial Sequence
  Description of Artificial SequenceHIF3a cDNA of splice variant 1
actcgtaactcgcacccgggtcctggctgcaccgcatcccctcctgcaccccctggatggcccttcagccaacgggggcctgggcgatggtcgaccacggagctgcgcaaggaaaagtcccgggatgcggcccgcagccggcgcagccaggagaccgaggtgctgtaccagctggctcacacgctgcccttcgcccgcggcgtcagcgcccacctggacaaggcctctatcatgcgcctcaccatcagctacctgcgcatgcaccgcctctgcgccgcagctggagctcattggacacagcatctttgatttcatccacccctgtgaccaagaggagcttcaggacgccctgaccccccagcagaccctgtccaggaggaaggtggaggcccccacggagcggtgcttctccttgcgcatgaagagtacactcaccagccgcgggcgcaccctcaacctcaaggcggccacctggaaggtgctgaactgctctggacatatgagggcctacaagccacctgcgcagacttctccagctgggagccctgactcagagcccccgctgcagtgcctggtgctcatctgcgaagccatcccccacccaggcagcctggagcccccactgggccgaggggccttcctcagccgccacagcctggacatgaagttcacctactgtgacgacaggattgcagaagtggctggctatagtcccgatgacctgatcggctgttccgcctacgagtacatccacgcgctggactccgatgcggtcagcaagagcatccacaccttgctgagcaagggccaggcagtaacagggcagtatcgcttcctggcccggagtggtggctacctgtggacccagacccaggccacagtggtgtcagggggacggggcccccagtcggagagtatcgtctgtgtccattttttaatcagccaggtggaagagaccggagtggtgctgtccctggagcaaacggagcaacactctcgcagacccattcagcggggcgccccctctcagaaggacacccctaaccctggggacagccttgacacccctggcccccggatccttgccttcctgcacccgccttccctgagcgaggctgccctggccgctgacccccgccgtttctgcagccctgacctccgtcgcctcctgggacccatcctggatggggcttcagtagcagccactcccagcaccccgctggccacacggcacccccaaagtcctctttcggctgatctcccagatgaactacctgtgggcaccgagaatgtgcacagactcttcacctccgggaaagacactgaggcagtggagacagatttagatatagctcaggaccccagcaccccactcctgaacctgaatgagcccctgggttttcactttgtcacccagtctggagtgcagtggcacaaacacagctcaccgcagcctcgacctcctgggctcaagtgatcctcctacttcagctcccacaagtagctgggactgcagctatgtgccatcatgcctggctgatgtttatatgttttgtagagacgaggtttcaccatgttgcccaggctggtcttgaactcctgagttcaagcgatccacctgccttggcctcccaaagtgctgggattactggtatgaaccaccacgcccgacagtaaatatgttttgaatgaataaactctcataaatg

SEQ ID NO: 7 - DNA, 2239 bp, Artificial Sequence
  Description of Artificial SequenceHIF3alpha cDNA of splice variant 2
tgggagcggcgactggcgagccatggcgctggggctgcagcgcgcaaggtcgaccacggagctgcgcaaggaaaagtcccgggatgcggcccgcagccggcgcagccaggagaccgaggtgctgtaccagctggctcacacgctgcccttcgcccgcggcgtcagcgcccacctggacaaggcctctatcatgcgcctcaccatcagctacctgcgcatgcaccgcctctgcgccgcaggggagtggaaccaggtgggagcagggggagaaccactggatgcctgctacctgaaggccctggagggcttcgtcatggtgctcaccgccgagggagacatggcttacctgtcggagaatgtcagcaaacacctgggcctcagtcagctggagctcattggacacagcatctttgatttcatccacccctgtgaccaagaggagcttcaggacgccctgaccccccagcagaccctgtccaggaggaaggtggaggcccccacggagcggtgcttctccttgcgcatgaagagtacgctcaccagccgcgggcgcaccctcaacctcaaggcggccacctggaaggtgctgaactgctctggacatatgagggcctacaagccacctgcgcagacttctccagctgggagccctgactcagagcccccgctgcagtgcctggtgctcatctgcgaagccatcccccacccaggcagcctggagcccccactgggccgaggggccttcctcagccgccacagcctggacatgaagttcacctactgtgacgacaggattgcagaagtggctggctatagtcccgatgacctgatcggctgttccgcctacgagtacatccacgcgctggactccgacgcggtcagcaagagcatccacaccttgctgagcaagggccaggcagtaacagggcagtatcgcttcctggcccggagtggtggctacctgtggacccagacccaggccacagtggtgtcagggggacggggcccccagtcggagagtatcgtctgtgtccattttttaatcaggtaagcaggaggaggggctggggtggctgtgtgtgggcctgatctgcatgtgtggacaggtgtgtgtgtgtgtgtgtgtgtgtgtgtgtgtgtgcgtatgagcatgcatgtgtatcatgcataagtgtatgtgagggagtgtgcacgtgtacacatatgaggaatgtgtgtcaccatgtaaatgccggtgtgtgtgtctgcatggacacaggtatgtgtatgggtgtgtagactgttaatttttttttttttttttttttgcgtgaacctctgcttaagtggattgttaattcaaattagaaaggggtctttatttggcctggcatggtggctcatgcctgtaatcctagcactttgggaggctgaggtgggcggattgcctgagctcaggagttcgaaaccagcctgggcaacatgacgaaatgctgtttctgctaataataccaaaaattagccgggtgtggtgacacatgcctgtgatcccaactactcgggaggctgaggcacgagaatcattagaacccgggtggtggaggctgcagtgagccgagattgcgtcagtgcactctggcctcggcaacagagcgagactctgtctcaaacaaacaaacaaacaaacaaaaggactctatattcaagttaaaataagaagtgtaacagaatcatggggtcttttttgctttttaaattttgatgtggctcacgcctgtaaatcccaaggtgttgggattacaggcgtgagccactgcacccggcccatgttgtggtttatatcagtagttcctttgtaaatagtgaacagtattccatggtatgaatagagcacagtttttttttttatccattcaccagttagaagacattgggctgtttccaagtttgggtgattacaaaaaacagctactgtaaacattctcatacaagattttatgagatcacatgttttcatttctcttgggtaaacagctaggattggaatggatgggttatatagtaagtgtatatttaatctaagaaactgccatggctgggcacagtggctcacgcctgtaatcccagtactttgggaagccaaggaaggaggatgactagagcctctgaggtgaagaccagcctgggcaaagtggttaagactcaaccgcaaaaaaagaaaaacagaaaacctgaaaacaaaccaaaaaaaaaaaaaaaa

SEQ ID NO: 8 - DNA, 2082 bp, Artificial Sequence
  Description of Artificial SequenceHIF3alpha cDNA of splice variant 3
gactggcgagccatggcgctggggctgcagcgcgcaaggtcgaccacggagctgcgcaaggaaaagtcccgggatgcggcccgcagccggcgcagccaggagaccgaggtgctgtaccagctggctcacacgctgcccttcgcccgcggcgtcagcgcccacctggacaaggcctctatcatgcgcctcaccatcagctacctgcgcatgcaccgcctctgcgccgcaggggagtggaaccaggtgggagcagggggagaaccactggatgcctgctacctgaaggccctggagggcttcgtcatggtgctcaccgccgagggagacatggcttacctgtcggagaatgtcagcaaacacctgggcctcagtcagctggagctcattggacacagcatctttgatttcatccacccctgtgaccaagaggagcttcaggacgccctgaccccccagcagaccctgtccaggaggaaggtggaggcccccacggagcggtgcttctccttgcgcatgaagagtacgctcaccagccgcgggcgcaccctcaacctcaaggcggccacctggaaggtgctgaactgctctggacatatgagggcctacaagccacctgcgcagacttctccagctgggagccctgactcagagcccccgctgcagtgcctggtgctcatctgcgaagccatcccccacccaggcagcctggagcccccactgggccgaggggccttcctcagccgccacagcctggacatgaagttcacctactgtgacgacaggattgcagaagtggctggctatagtcccgatgacctgatcggctgttccgcctacgagtacatccacgcgctggactccgacgcggtcagcaagagcatccacaccttgctgagcaagggccaggcagtaacagggcagtatcgcttcctggcccggagtggtggctacctgtggacccagacccaggccacagtggtgtcagggggacggggcccccagtcggagagtatcgtctgtgtccattttttaatcagccaggtggaagagaccggagtggtgctgtccctggagcaaacggagcaacactctcgcagacccattcagcggggcgccccctctcagaaggacacccctaaccctggggacagccttgacacccctggcccccggatccttgccttcctgcacccgccttccctgagcgaggctgccctggccgctgacccccgccgtttctgcagccctgacctccgtcgcctcctgggacccatcctggatggggcttcagtagcagccactcccagcaccccgctggccacacggcacccccaaagtcctctttcggctgatctcccagatgaactacctgtgggcaccgagaatgtgcacagactcttcacctccgggaaagacactgaggcagtggagacagatttagatatagctcaggatgctgatgctctggatttggagatgctggccccctacatctccatggatgatgacttccagctcaacgccagcgagcagctacccagggcctaccacagacctctgggggctgtcccccggccccgtgctcggagcttccatggcctgtcacctccagcccttgagccctccctgctaccccgctgggggagtgacccccggctgagctgctccagcccttccagaggggacccctcagcatcctctcccatggctggggctcggaagaggaccctggcccagagctcagaggacgaggacgagggagtggagctgctgggagtgagacctcccaaaaggtcccccagcccagaacacgaaaactttctgctctttcctctcagcctggtgtgttgggggattaatgggattctctggccctcattacctagctggcttaaacctactgttttatagataggaaaccagagaggggcaggggctggttgagggtcatacagaaagtcagtgggccagctgagactaaagcctgatcttctagtttcactaatgggtattaaaaacctctgcagtgaactgagattgcgccactgcaccccagcatgagcgacagaatgggaccttgtc

SEQ ID NO: 9 - DNA, 2595 bp, Artificial Sequence
  Description of Artificial SequenceHIF3alpha cDNA of splice variant 5
aactcgcacccgggtcctggctgcaccgcatcccctcctgcaccccctggatggcccttcagccaacgggggcctgggcgatggtcgaccacggagctgcgcaaggaaaagtcccgggatgcggcccgcagccggcgcagccaggagaccgaggtgctgtaccagctggctcacacgctgcccttcgcccgcggcgtcagcgcccacctggacaaggcctctatcatgcgcctcaccatcagctacctgcgcatgcaccgcctctgcgccgcaggggagtggaaccaggtgggagcagggggagaaccactggatgcctgctacctgaaggccctggagggcttcgtcatggtgctcaccgccgagggagacatggcttacctgtcggagaatgtcagcaaacacctgggcctcagtcagctggagctcattggacacagcatctttgatttcatccacccctgtgaccaagaggagcttcaggacgccctgaccccccagcagaccctgtccaggaggaaggtggaggcccccacggagcggtgcttctccttgcgcatgaagagtacgctcaccagccgcgggcgcaccctcaacctcaaggcggccacctggaaggtgctgaactgctctggacatatgagggcctacaagccacctgcgcagacttctccagctgggagccctgactcagagcccccgctgcagtgcctggtgctcatctgcgaagccatcccccacccaggcagcctggagcccccactgggccgaggggccttcctcagccgccacagcctggacatgaagttcacctactgtgacgacaggattgcagaagtggctggctatagtcccgatgacctgatcggctgttccgcctacgagtacatccacgcgctggactccgacgcggtcagcaagagcatccacaccttgctgagcaagggccaggcagtaacagggcagtatcgcttcctggcccggagtggtggctacctgtggacccagacccaggccacagtggtgtcagggggacggggcccccagtcggagagtatcgtctgtgtccattttttaatcagccaggtggaagagaccggagtggtgctgtccctggagcaaacggagcaacactctcgcagacccattcagcggggcgccccctctcagaaggacacccctaaccctggggacagccttgacacccctggcccccggatccttgccttcctgcacccgccttccctgagcgaggctgccctggccgctgacccccgccgtttctgcagccctgacctccgtcgcctcctgggacccatcctggatggggcttcagtagcagccactcccagcaccccgctggccacacggcacccccaaagtcctctttcggctgatctcccagatgaactacctgtgggcaccgagaatgtgcacagactcttcacctccgggaaagacactgaggcagtggagacagatttagatatagctcaggatgctgatgctctggatttggagatgctggccccctacatctccatggatgatgacttccagctcaacgccagcgagcagctacccagggcctaccacagacctctgggggctgtcccccggccccgtgctcggagcttccatggcctgtcacctccagcccttgagccctccctgctaccccgctgggggagtgacccccggctgagctgctccagcccttccagaggggacccctcagcatcctctcccatggctggggctcggaagaggaccctggcccagagctcagaggacgaggacgagggagtggagctgctgggagtgagacctcccaaaaggtcccccagcccagaacacgaaaactttctgctctttcctctcagcctgagtttccttctgacaggaggaccagccccagggagcctgcaggaccccactgaacttacccaattccttctttcagtcttaagttttcccattctagacccctaccctctaggctgtgctgctcctggacttcatgcctctccattctcattgcctacaatctctgtgccccagaaccccctccacttcccaccccagccctccagacatgcacttaccttgactttaccccacatgtttggggcacctggggctccctcaccccttgggtggtttgcaatctgaagacttctccagccacacaggcacatgcacaggcacggtgctgtctgcatattgccaggtggggagagaagccaggacccctcagctgtctgccaccatctatgtgcctcccttaccccccagctttctttctacagatggtgctactcttggtctcccacaggaaaaggcctccccccttcttagccccatttaccccgtttgtggaaggcactgctcgctctgttttgtcagagagtggcctatccagattggtgctatgggggggtctgacccctccctcctccctctggaggtgatgtgggccctcaatggagggaattgtgctgggctagggaaaggggagggactagactggccacactggctctgaaactcaccaatctctatacaccataaagacctcaccttggtaggcaccag

SEQ ID NO: 10 - DNA, 23 bp, Artificial Sequence
  Description of Artificial Sequenceprimer for HIF3a splice variant 1
gggctcaagtgatcctcctactt

SEQ ID NO: 11 - DNA, 22 bp, Artificial Sequence
  Description of Artificial Sequenceprimer for HIF3a splice variant 1
catgatggcacatagctgcagt

SEQ ID NO: 12 - DNA, 22 bp, Artificial Sequence
  Description of Artificial Sequenceprimer for HIF3a splice variant 2
tttgcgtgaacctctgcttaag

SEQ ID NO: 13 - DNA, 18 bp, Artificial Sequence
  Description of Artificial Sequenceprimer for HIF3a splice variant 2
caccatgccaggccaaat

SEQ ID NO: 14 - DNA, 23 bp, Artificial Sequence
  Description of Artificial Sequenceprimer for HIF3a splice variant 3
tctctggccctcattacctagct

SEQ ID NO: 15 - DNA, 21 bp, Artificial Sequence
  Description of Artificial Sequenceprimer for HIF3a splice variant 3
ctgtatgaccctcaaccagcc

SEQ ID NO: 16 - DNA, 22 bp, Artificial Sequence
  Description of Artificial Sequenceprimer for HIF3a splice variant 5
actcttggtctcccacaggaaa

SEQ ID NO: 17 - DNA, 20 bp, Artificial Sequence
  Description of Artificial Sequenceprimer for HIF3a splice variant 5
aacagagcgagcagtgcctt

SEQ ID NO: 18 - DNA, 20 bp, Artificial Sequence
  Description of Artificial Sequenceprimer for the cyclophilin B gene
actgaagcactacgggcctg

SEQ ID NO: 19 - DNA, 19 bp, Artificial Sequence
  Description of Artificial Sequenceprimer for the cyclophilin B gene
agccgttggtgtctttgcc

SEQ ID NO: 20 - DNA, 20 bp, Artificial Sequence
  Description of Artificial Sequenceprimer for the gene of the ribosomal protein S9
ggtcaaatttaccctggcca

SEQ ID NO: 21 - DNA, 22 bp, Artificial Sequence
  Description of Artificial Sequenceprimer for the gene of the ribosomal protein S9
tctcatcaagcgtcagcagttc

SEQ ID NO: 22 - DNA, 19 bp, Artificial Sequence
  Description of Artificial Sequenceprimer for the beta-actin gene
tggaacggtgaaggtgaca

SEQ ID NO: 23 - DNA, 19 bp, Artificial Sequence
  Description of Artificial Sequenceprimer for the beta-actin gene
ggcaagggacttcctgtaa

SEQ ID NO: 24 - DNA, 20 bp, Artificial Sequence
  Description of Artificial Sequenceprimer for the GAPDH gene
cgtcatgggtgtgaaccatg

SEQ ID NO: 25 - DNA, 21 bp, Artificial Sequence
  Description of Artificial Sequenceprimer for the GAPDH gene
gctaagcagttggtggtgcag

SEQ ID NO: 26 - DNA, 21 bp, Artificial Sequence
  Description of Artificial Sequenceprimer for the transferrin receptor gene
gtcgctggtcagttcgtgatt

SEQ ID NO: 27 - DNA, 23 bp, Artificial Sequence
  Description of Artificial Sequenceprimer for the transferrin receptor gene
agcagttggctgttgtacctctc

SEQ ID NO: 28 - DNA, 1353 bp, Homo sapiens
atgcggcccgcagccggcgcagccaggagaccgaggtgctgtaccagctggctcacacgctgcccttcgcccgcggcgtcagcgcccacctggacaaggcctctatcatgcgcctcaccatcagctacctgcgcatgcaccgcctctgcgccgcagctggagctcattggacacagcatctttgatttcatccacccctgtgaccaagaggagcttcaggacgccctgaccccccagcagaccctgtccaggaggaaggtggaggcccccacggagcggtgcttctccttgcgcatgaagagtacactcaccagccgcgggcgcaccctcaacctcaaggcggccacctggaaggtgctgaactgctctggacatatgagggcctacaagccacctgcgcagacttctccagctgggagccctgactcagagcccccgctgcagtgcctggtgctcatctgcgaagccatcccccacccaggcagcctggagcccccactgggccgaggggccttcctcagccgccacagcctggacatgaagttcacctactgtgacgacaggattgcagaagtggctggctatagtcccgatgacctgatcggctgttccgcctacgagtacatccacgcgctggactccgatgcggtcagcaagagcatccacaccttgctgagcaagggccaggcagtaacagggcagtatcgcttcctggcccggagtggtggctacctgtggacccagacccaggccacagtggtgtcagggggacggggcccccagtcggagagtatcgtctgtgtccattttttaatcagccaggtggaagagaccggagtggtgctgtccctggagcaaacggagcaacactctcgcagacccattcagcggggcgccccctctcagaaggacacccctaaccctggggacagccttgacacccctggcccccggatccttgccttcctgcacccgccttccctgagcgaggctgccctggccgctgacccccgccgtttctgcagccctgacctccgtcgcctcctgggacccatcctggatggggcttcagtagcagccactcccagcaccccgctggccacacggcacccccaaagtcctctttcggctgatctcccagatgaactacctgtgggcaccgagaatgtgcacagactcttcacctccgggaaagacactgaggcagtggagacagatttagatatagctcaggaccccagcaccccactcctgaacctgaatgagcccctgggttttcactttgtcacccagtctggagtgcagtggcacaaacacagctcaccgcagcctcgacctcctgggctcaagtga

SEQ ID NO: 29 - DNA, 1029 bp, Homo sapiens
atggcgctggggctgcagcgcgcaaggtcgaccacggagctgcgcaaggaaaagtcccgggatgcggcccgcagccggcgcagccaggagaccgaggtgctgtaccagctggctcacacgctgcccttcgcccgcggcgtcagcgcccacctggacaaggcctctatcatgcgcctcaccatcagctacctgcgcatgcaccgcctctgcgccgcaggggagtggaaccaggtgggagcagggggagaaccactggatgcctgctacctgaaggccctggagggcttcgtcatggtgctcaccgccgagggagacatggcttacctgtcggagaatgtcagcaaacacctgggcctcagtcagctggagctcattggacacagcatctttgatttcatccacccctgtgaccaagaggagcttcaggacgccctgaccccccagcagaccctgtccaggaggaaggtggaggcccccacggagcggtgcttctccttgcgcatgaagagtacgctcaccagccgcgggcgcaccctcaacctcaaggcggccacctggaaggtgctgaactgctctggacatatgagggcctacaagccacctgcgcagacttctccagctgggagccctgactcagagcccccgctgcagtgcctggtgctcatctgcgaagccatcccccacccaggcagcctggagcccccactgggccgaggggccttcctcagccgccacagcctggacatgaagttcacctactgtgacgacaggattgcagaagtggctggctatagtcccgatgacctgatcggctgttccgcctacgagtacatccacgcgctggactccgacgcggtcagcaagagcatccacaccttgctgagcaagggccaggcagtaacagggcagtatcgcttcctggcccggagtggtggctacctgtggacccagacccaggccacagtggtgtcagggggacggggcccccagtcggagagtatcgtctgtgtccattttttaatcaggtaa

SEQ ID NO: 30 - DNA, 1899 bp, Homo sapiens
atggcgctggggctgcagcgcgcaaggtcgaccacggagctgcgcaaggaaaagtcccgggatgcggcccgcagccggcgcagccaggagaccgaggtgctgtaccagctggctcacacgctgcccttcgcccgcggcgtcagcgcccacctggacaaggcctctatcatgcgcctcaccatcagctacctgcgcatgcaccgcctctgcgccgcaggggagtggaaccaggtgggagcagggggagaaccactggatgcctgctacctgaaggccctggagggcttcgtcatggtgctcaccgccgagggagacatggcttacctgtcggagaatgtcagcaaacacctgggcctcagtcagctggagctcattggacacagcatctttgatttcatccacccctgtgaccaagaggagcttcaggacgccctgaccccccagcagaccctgtccaggaggaaggtggaggcccccacggagcggtgcttctccttgcgcatgaagagtacgctcaccagccgcgggcgcaccctcaacctcaaggcggccacctggaaggtgctgaactgctctggacatatgagggcctacaagccacctgcgcagacttctccagctgggagccctgactcagagcccccgctgcagtgcctggtgctcatctgcgaagccatcccccacccaggcagcctggagcccccactgggccgaggggccttcctcagccgccacagcctggacatgaagttcacctactgtgacgacaggattgcagaagtggctggctatagtcccgatgacctgatcggctgttccgcctacgagtacatccacgcgctggactccgacgcggtcagcaagagcatccacaccttgctgagcaagggccaggcagtaacagggcagtatcgcttcctggcccggagtggtggctacctgtggacccagacccaggccacagtggtgtcagggggacggggcccccagtcggagagtatcgtctgtgtccattttttaatcagccaggtggaagagaccggagtggtgctgtccctggagcaaacggagcaacactctcgcagacccattcagcggggcgccccctctcagaaggacacccctaaccctggggacagccttgacacccctggcccccggatccttgccttcctgcacccgccttccctgagcgaggctgccctggccgctgacccccgccgtttctgcagccctgacctccgtcgcctcctgggacccatcctggatggggcttcagtagcagccactcccagcaccccgctggccacacggcacccccaaagtcctctttcggctgatctcccagatgaactacctgtgggcaccgagaatgtgcacagactcttcacctccgggaaagacactgaggcagtggagacagatttagatatagctcaggatgctgatgctctggatttggagatgctggccccctacatctccatggatgatgacttccagctcaacgccagcgagcagctacccagggcctaccacagacctctgggggctgtcccccggccccgtgctcggagcttccatggcctgtcacctccagcccttgagccctccctgctaccccgctgggggagtgacccccggctgagctgctccagcccttccagaggggacccctcagcatcctctcccatggctggggctcggaagaggaccctggcccagagctcagaggacgaggacgagggagtggagctgctgggagtgagacctcccaaaaggtcccccagcccagaacacgaaaactttctgctctttcctctcagcctggtgtgttgggggattaatgggattctctggccctcattacctagctggcttaaacctactgttttatag

SEQ ID NO: 31 - DNA, 1947 bp, Homo sapiens
atgcgcctcaccatcagctacctgcgcatgcaccgcctctgcgccgcaggggagtggaaccaggtgggagcagggggagaaccactggatgcctgctacctgaaggccctggagggcttcgtcatggtgctcaccgccgagggagacatggcttacctgtcggagaatgtcagcaaacacctgggcctcagtcagctggagctcattggacacagcatctttgatttcatccacccctgtgaccaagaggagcttcaggacgccctgaccccccagcagaccctgtccaggaggaaggtggaggcccccacggagcggtgcttctccttgcgcatgaagagtacgctcaccagccgcgggcgcaccctcaacctcaaggcggccacctggaaggtgctgaactgctctggacatatgagggcctacaagccacctgcgcagacttctccagctgggagccctgactcagagcccccgctgcagtgcctggtgctcatctgcgaagccatcccccacccaggcagcctggagcccccactgggccgaggggccttcctcagccgccacagcctggacatgaagttcacctactgtgacgacaggattgcagaagtggctggctatagtcccgatgacctgatcggctgttccgcctacgagtacatccacgcgctggactccgacgcggtcagcaagagcatccacaccttgctgagcaagggccaggcagtaacagggcagtatcgcttcctggcccggagtggtggctacctgtggacccagacccaggccacagtggtgtcagggggacggggcccccagtcggagagtatcgtctgtgtccattttttaatcagccaggtggaagagaccggagtggtgctgtccctggagcaaacggagcaacactctcgcagacccattcagcggggcgccccctctcagaaggacacccctaaccctggggacagccttgacacccctggcccccggatccttgccttcctgcacccgccttccctgagcgaggctgccctggccgctgacccccgccgtttctgcagccctgacctccgtcgcctcctgggacccatcctggatggggcttcagtagcagccactcccagcaccccgctggccacacggcacccccaaagtcctctttcggctgatctcccagatgaactacctgtgggcaccgagaatgtgcacagactcttcacctccgggaaagacactgaggcagtggagacagatttagatatagctcaggatgctgatgctctggatttggagatgctggccccctacatctccatggatgatgacttccagctcaacgccagcgagcagctacccagggcctaccacagacctctgggggctgtcccccggccccgtgctcggagcttccatggcctgtcacctccagcccttgagccctccctgctaccccgctgggggagtgacccccggctgagctgctccagcccttccagaggggacccctcagcatcctctcccatggctggggctcggaagaggaccctggcccagagctcagaggacgaggacgagggagtggagctgctgggagtgagacctcccaaaaggtcccccagcccagaacacgaaaactttctgctctttcctctcagcctgagtttccttctgacaggaggaccagccccagggagcctgcaggaccccactgaacttacccaattccttctttcagtcttaagttttcccattctagacccctaccctctaggctgtgctgctcctggacttcatgcctctccattctcattgcctacaatctctgtgccccagaaccccctccacttcccaccccagccctccagacatgcacttaccttgactttaccccacatgtttggggcacctggggctccctcaccccttgggtggtttgcaatctga